(19) United States
(12) Patent Application Publication
(10) Pub. No.: US 2008/0242681 A1
(43) Pub. Date: Oct. 2, 2008

(54) N-4-(6-(Hetero)Aryl-Pyrimidin-4-Ylaminophenyl)-Benzenesulfonamides as Kinase Inhibitors

(75) Inventors: Johannes Barsig, Konstanz (DE); Monika Baudler, Konstanz (DE); Daniela Bundschuh, Ermatingen (CH); Florian Gantner, Ermatingen (CH); Ulrich Graedler, Heidelberg (DE); Isabelle Heit, Winterthur (CH); Thomas Martin, Konstanz (DE); Michaela Schaefer, Orsingen-Nenzingen (DE); Imre Schlemminger, Konstanz (DE); Josef Stadlwieser, Konstanz (DE); Wolf-Ruediger Ulrich, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/586,121

(22) PCT Filed: Jan. 19, 2005
(86) PCT No.: PCT/EP2005/050206

§ 371 Date: Aug. 25, 2006

(30) Foreign Application Priority Data
Jan. 22, 2004 (EP) 04001310.4

(51) Int. Cl. A61K 31/505; C07D 239/42; A61P 31/00; A61P 35/00; A61P 25/28; A61P 3/10; A61P 9/10; A61P 19/02; A61P 29/00; A61P 17/00; A61P 27/02

(52) U.S. Cl. 514/256; 544/327

(57) ABSTRACT

Compounds of a certain formula 1 in which R1 and R2 have the meanings indicated in the description are novel kinase inhibitors. Formula (1), in which R1 is phenyl, phenyl substituted by R3 and/or R4, naphthalenyl, naphthalenyl substituted by and/or R6, aryl1, aryl1 substituted by R7 and/or R8, R9, R10 or R11, R2 is phenyl, phenyl substituted by R12 and/or R13, naphthalenyl, naphthalenyl substituted by R14 and/or R15, aryl2, aryl2 substituted by R16 and/or R17 or a radical selected from Formula (2).

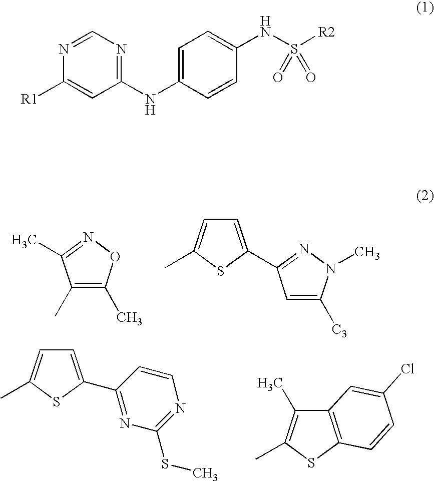

### FIELD OF APPLICATION OF THE INVENTION

The invention relates to novel kinase inhibitors, which are used in the pharmaceutical industry for the production of pharmaceutical compositions.

### KNOWN TECHNICAL BACKGROUND

In the International patent application WO02/12198 4-pyrimidineamine derivatives with neuroprotective properties are described.

### DESCRIPTION OF THE INVENTION

It has now been found that the kinase inhibitors, which are described in greater details below, have surprising and particularly advantageous properties.

The invention thus relates to compounds of formula 1

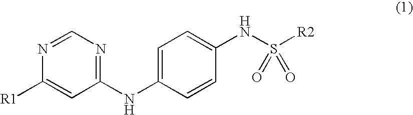

in which
R1 is phenyl,
   phenyl substituted by R3 and/or R4,
   naphthalenyl,
   naphthalenyl substituted by R5 and/or R6,
   aryl1,
   aryl1 substituted by R7 and/or R8,
   R9,
   R10 or
   R11,
R2 is phenyl,
   phenyl substituted by R12 and/or R13,
   naphthalenyl,
   naphthalenyl substituted by R14 and/or R15,
   aryl2,
   aryl2 substituted by R16 and/or R17
   or a radical selected from

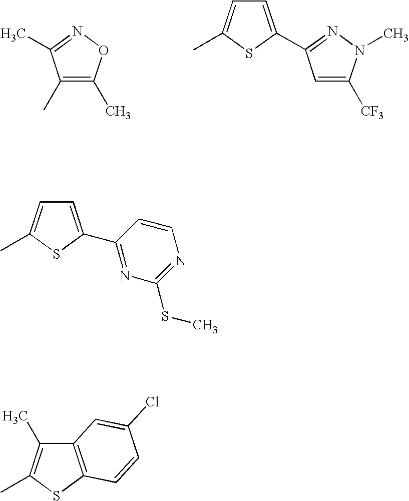

R3 is hydroxyl, halogen, cyano, carboxyl, nitro, 1-4C-alkyl, trifluoromethyl, 1-4C-alkoxy, 1-4C-alkoxy completely or predominately substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32 wherein
   R31 is hydrogen, 1-4C-alkyl or 1-4C-alkoxy-2-4C-alkyl, and
   R32 is hydrogen, 1-4C-alkyl or 1-4C-alkoxy-2-4C-alkyl or wherein
   R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   n is an integer from 0 to 4,
   m is an integer from 2 to 4,
R4 is halogen, cyano, nitro, 1-4C-alkyl, trifluoromethyl, 1-4C-alkoxy or 1-4C-alkoxy completely or pre-dominantly substituted by fluorine, amino or mono- or di-1-4C-alkylamino,
R5 is hydroxyl, halogen, cyano, carboxyl, nitro, 1-4C-alkyl, trifluoromethyl, 1-4C-alkoxy, 1-4C-alkoxy completely or predominately substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R6 is halogen, cyano, nitro, 1-4C-alkyl, trifluoromethyl, 1-4C-alkoxy or 1-4C-alkoxy completely or pre-dominantly substituted by fluorine, amino or mono- or di-1-4C-alkylamino,
Aryl1 furanyl, thiophenyl, oxazolyl, isoxazolyl, thiazolyl, isothiazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl or dibenzofuranyl,
R7 is hydroxyl, halogen, cyano, carboxyl, nitro, 1-4C-alkyl, trifluoromethyl, 1-4C-alkoxy, 1-4C-alkoxy completely or predominately substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R8 is halogen, cyano, nitro, 1-4C-alkyl, trifluoromethyl, 1-4C-alkoxy or 1-4C-alkoxy completely or pre-dominantly substituted by fluorine, amino or mono- or di-1-4C-alkylamino,
R9 is unsubstituted pyrrolyl, pyrazolyl, imidazolyl, indolyl, indazolyl, benzimidazolyl or benztriazolyl, or a radical selected from

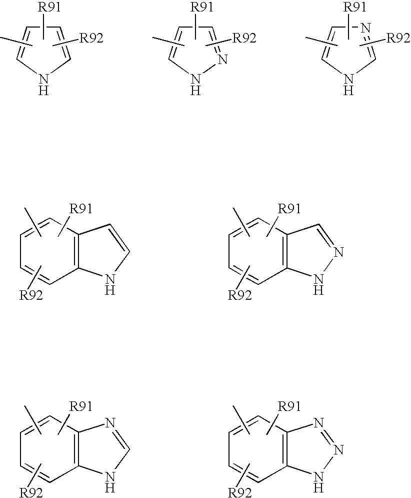

wherein
R91 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R92 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R10 is a radical selected from

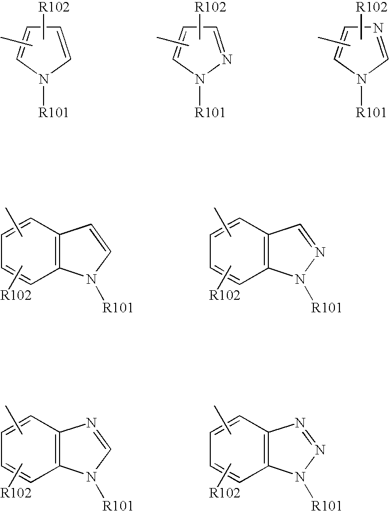

wherein
R101 is 1-4C-alkyl, 2,2,2-trifluoroethyl or 3,3,3-trifluoropropyl,
R102 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R11 is a radical selected from

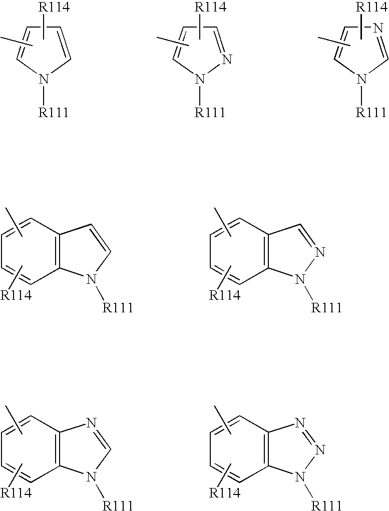

wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, —(CH₂)ₚ—N(R112)R113 or —CH₂CH(OH)CH₂N(R112)R113, wherein
   R112 is hydrogen, 1-4C-alkyl or 1-4C-alkoxy-2-4C-alkyl, and
   R113 is hydrogen, 1-4C-alkyl or 1-4C-alkoxy-2-4C-alkyl, or wherein
   R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan, 4-(1-4C-alkyl)-[1,4]diazepan-, morpholin-, thiomorpholin- or an azepan-ring,
   p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds with the proviso that the following compounds are excluded
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide.

Halogen within the meaning of the present invention is bromine, chlorine or fluorine.

1-4C-Alkyl is a straight-chain or branched alkyl radical having 1 to 4 carbon atoms. Examples are the butyl, isobutyl, sec-butyl, tert-butyl, propyl, isopropyl, ethyl and methyl radicals.

1-4C-Alkoxy is a radical which, in addition to the oxygen atom, contains a straight-chain or branched alkyl radical having 1 to 4 carbon atoms. Alkoxy radicals having 1 to 4 carbon atoms which may be mentioned in this context are, for example, the butoxy, isobutoxy, sec-butoxy, tert-butoxy, propoxy, isopropoxy, ethoxy and methoxy radicals.

1-4C-Alkoxy which is completely or predominantly substituted by fluorine is, for example, the 2,2,3,3,3-pentafluoropropoxy, the perfluoroethoxy, the 1,2,2-trifluoroethoxy and in particular the 1,1,2,2-tetrafluoroethoxy, the 2,2,2-trifluoroethoxy, the trifluoromethoxy and the difluoromethoxy radical, of which the difluoromethoxy radical is preferred. “Predominantly” in this connection means that more than half of the hydrogen atoms of the 1-4C-alkoxy groups are replaced by fluorine atoms.

1-4C-Alkoxycarbonyl is a carbonyl group to which one of the abovementioned 1-4C-alkoxy radicals is bonded. Examples are the methoxycarbonyl [CH₃O—C(O)—] and the ethoxycarbonyl [CH₃CH₂O—C(O)—] radical.

1-4C-Alkylcarbonyl is a carbonyl group to which one of the abovementioned 1-4C-alkyl radicals is bonded. An example is the acetyl radical [CH₃CO—].

Mono- or Di-1-4C-alkylamino radicals contain in addition to the nitrogen atom, one or two of the above-mentioned 1-4C-alkyl radicals. Preferred are the di-1-4C-alkylamino radicals, especially the dimethylamino, the diethylamino and the diisopropylamino radical.

2-4C-Alkyl is a straight-chain or branched alkyl radical having 2 to 4 carbon atoms. Examples are the butyl, isobutyl, sec-butyl, tert-butyl, propyl, isopropyl and ethyl radicals.

1-4C-Alkoxy-2-4C-alkyl stands for one of the abovementioned 2-4C-alkyl radicals which is substituted by one of the abovementioned 1-4C-alkoxy radicals. Examples which may be mentioned are the 2-methoxyethyl and the 3-methoxypropyl radical.

Mono- or di-1-4C-alkylaminocarbonyl radicals are, for example, the methylaminocarbonyl, the dimethylaminocarbonyl and the diethylaminocarbonyl radicals.

An 1-4C-alkylcarbonylamino radical is, for example, the propionylamino [C₃H₇C(O) NH—] and the acetylamino radical [CH₃C(O)NH—].

—(CH₂)ₙ—, —(CH₂)ₘ— or —(CH₂)ₚ— stands for a straight-chain or branched alkylene radical having, n, m or p carbon atoms. Examples, which may be mentioned are methylene (—CH₂—), ethylene (—CH₂—CH₂—), trimethylene (—CH₂—CH₂—CH₂—) or tetramethylene (—CH₂—CH₂—CH₂—CH₂—).

In case R9 is a radical selected from

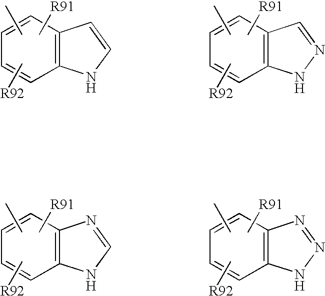

R91, R92 and the bond to the pyrimidinyl-ring can be attached to any carbon atom of the condensed ring systems with a free valence.

In case R10 is a radical selected from

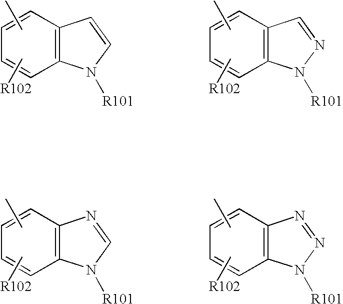

R102 and the bond to the pyrimidinyl-ring can be attached to any carbon atom of the condensed ring systems with a free valence. Preferred are those cases, wherein R10 is a indol-5-yl or indazol-5-yl radical, and R102 is attached in 3-position or wherein R10 is a indol-3-yl or indazol-3-yl radical and R102 is attached in 5-position.

In case R11 is a radical selected from

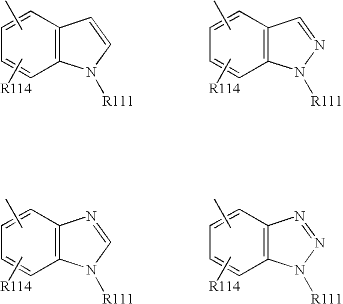

R114 and the bond to the pyrimidinyl-ring can be attached to any carbon atom of the condensed ring systems with a free valence. Preferred are those cases, wherein R11 is a indol-5-yl or indazol-6-yl radical.

Suitable salts for compounds of the formula 1—depending on substitution—are all acid addition salts or all salts with bases. Particular mention may be made of the pharmacologically tolerable inorganic and organic acids and bases customarily used in pharmacy. Those suitable are, on the one hand, water-soluble and water-insoluble acid addition salts with acids such as, for example, hydrochloric acid, hydrobromic acid, phosphoric acid, nitric acid, sulphuric acid, formic acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds according to the invention on an industrial scale, are converted into pharmacologically tolerable salts by processes known to the person skilled in the art.

According to expert's knowledge the compounds of the invention as well as their salts may contain, e.g. when isolated in crystalline form, varying amounts of solvents. Included within the scope of the invention are therefore all solvates and in particular all hydrates of the compounds of formula 1 as well as all solvates and in particular all hydrates of the salts of the compounds of formula 1.

An embodiment (embodiment A) of the compounds of formula 1 are those in which
R1 is phenyl,
   phenyl substituted by R3 and/or R4,
   naphthalenyl or
   naphthalenyl substituted by R5 and/or R6,
R2 is phenyl,
   phenyl substituted by R12 and/or R13,
   naphthalenyl,
   naphthalenyl substituted by R14 and/or R15,
   aryl2,
   aryl2 substituted by R16 and/or R17
   or a radical selected from

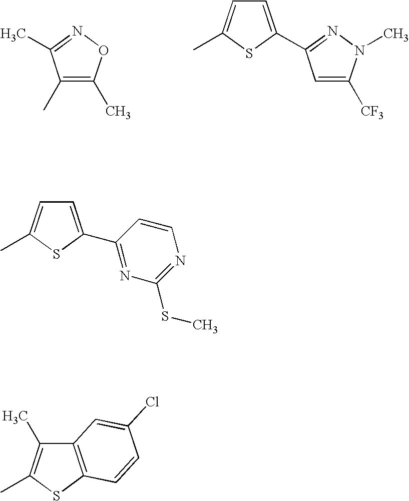

R3 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy or benzyloxy,
R4 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R5 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or 1-4C-alkylaminocarbonyl, 1-4C-alkylcarbonylamino, phenoxy or benzyloxy,
R6 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 14C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds with the proviso that the following compounds are excluded
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide, and
N-{4-[6-(3-Dimethylamino-phenyl)-pyrimidin-4-ylamino]-phenyl}-4-methyl-benzenesulfonamide.

Compounds of formula 1 of embodiment A to be emphasized are those in which
R1 is phenyl, 3-fluorophenyl, 4-fluorophenyl, 3-methoxyphenyl, 4-methoxyphenyl, 3,4-dimethoxyphenyl, 3-acetylphenyl, 4-acetylphenyl, 3-cyanophenyl, 4-phenoxyphenyl or naphthalen-1-yl,
R2 is phenyl, phenyl substituted by R12 and/or R13, thiophenyl, naphthalenyl or 5-(1-methyl-5-trifluoromethyl-1H-pyrazol-3-yl)-thiophene-2-yl,
R12 is halogen, cyano, nitro, 1-4C-alkyl, trifluoromethyl, 1-4C-alkoxy, 1-4C-alkoxy which is completely or predominantly substituted by fluorine or 1-4C-alkoxycarbonyl,
R13 is halogen, 1-4C-alkyl or 1-4C-alkoxy,
   and the salts of these compounds with the proviso that the following compounds are excluded
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide, and
N-{4-[6-(3,4-Dimethoxy-phenyl)-pyrimidin-4-ylamino]-phenyl}-4-methyl-benzenesulfonamide.

Preferred compounds of formula 1 of embodiment A are those in which
R1 is phenyl, 3-fluorophenyl, 4-fluorophenyl, 3-methoxyphenyl, 4-methoxyphenyl, 4-acetylphenyl or naphthalen-1-yl,
R2 is 2-fluorophenyl, 3-fluorophenyl, 3,4-difluorophenyl, 2,4-difluorophenyl, 2,6-difluorophenyl, 3,4-dichlorophenyl, 3-chloro-4-fluorophenyl, 3-chloro-4-methylphenyl, 3-bromophenyl, 3-methylphenyl, 4-methylphenyl, 4-isopropylphenyl, 4-trifluoromethylphenyl, 4-methoxyphenyl, 4-cyanophenyl or 5-(1-methyl-5-trifluoromethyl-1H-pyrazol-3-yl)-thiophene-2-yl,
   and the salts of these compounds with the proviso that the following compounds are excluded
4-Methyl-N-[4-(6-phenyl-pyrimidin-4-ylamino)-phenyl]-benzenesulfonamide and
4-Methyl-N-[4-(6-naphthalen-1-yl-pyrimidin-4-ylamino)-phenyl]-benzenesulfonamide.

Another embodiment (embodiment B) of the compounds of formula 1 are those in which
R1 is phenyl substituted by R3 and/or R4 or naphthalenyl substituted by R5 and/or R6,
R2 is phenyl,
   phenyl substituted by R12 and/or R13,
   naphthalenyl,
   naphthalenyl substituted by R14 and/or R15,
   aryl2,
   aryl2 substituted by R16 and/or R17
   or a radical selected from

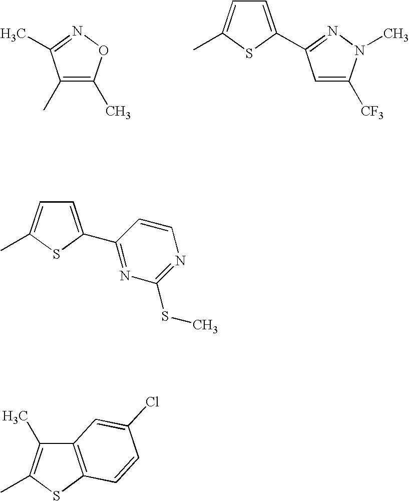

R3 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
   R31 is hydrogen, 1-4C-alkyl or 1-4C-alkoxy-2-4C-alkyl, and
   R32 is hydrogen, 1-4C-alkyl or 1-4C-alkoxy-2-4C-alkyl or wherein
   R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   n is an integer from 0 to 4,
   m is an integer from 2 to 4,
R4 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R5 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R₃₁)R₃₂,
R6 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Compounds of formula 1 of embodiment B to be emphasized are those in which
R1 is phenyl substituted in para or meta position by R3 or phenyl substituted in para and meta position by R3 and R4,
R2 is phenyl substituted by R12 and/or R13,
R3 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
   R31 is hydrogen, 1-4C-alkyl or 1-4C-alkoxy-2-4C-alkyl, and
   R32 is hydrogen, 1-4C-alkyl or 1-4C-alkoxy-2-4C-alkyl or wherein
   R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   n is an integer from 1 to 4,
   m is an integer from 2 to 4,
R4 is fluorine, methyl or methoxy,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Further compounds of formula 1 of embodiment B to be emphasized are those in which
R1 is phenyl substituted in para or meta position by R3,
R2 is phenyl substituted by R12 and/or R13,
R3 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
   R31 is hydrogen, 1-4C-alkyl or 1-4C-alkoxy-2-4C-alkyl, and
   R32 is hydrogen, 1-4C-alkyl or 1-4C-alkoxy-2-4C-alkyl or wherein
   R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   n is an integer from 1 to 4,
   m is an integer from 2 to 4,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Compounds of formula 1 of embodiment B particularly to be emphasized are those in which
R1 is phenyl substituted in para position by R3 or phenyl substituted in para position by R3 and in meta position by R4,
R2 is phenyl substituted by R12 and/or R13,
R3 is morpholin-4-ylmethyl, morpholin-4-ylethyl, morpholin-4-ylpropyl, 2-morpholin-4-ylethoxy, 3-morpholin-4-ylpropoxy, 4-methylpiperazin-1-ylmethyl, 4-methylpiperazin-1-ylethyl, 4-methylpiperazin-1-ylpropyl, 2-(4-methylpiperazin-1-yl)ethoxy, 3-(4-methylpiperazin-1-yl)propoxy, 1-methylpiperidin-4-ylmethoxy, 1-methylpiperidin-4-ylmethyl, 1-methylpiperidin-4-ylethyl, 1-methylpiperidin-4-ylpropyl, 2-(1-methylpiperidin-4-yl)ethoxy, 3-(1-methylpiperidin-4-yl)propoxy, pyrrolidin-1-ylpropyl, pyrrolidin-1-ylethyl, pyrrolidin-1-ylmethyl, 3-pyrrolidin-1-ylpropoxy, 2-pyrrolidin-1-ylethoxy, piperidin-1-ylmethyl, piperidin-1-ylethyl, piperidin-1-ylpropyl, 2-(piperidin-1-yl)ethoxy, 3-(piperidin-1-yl)propoxy,
R4 is fluorine, methyl or methoxy,
R12 is fluorine, chlorine, cyano, methyl, isopropyl, trifluoromethyl or methoxy,
R13 is fluorine or chlorine,
   and the salts of these compounds.

Further compounds of formula 1 of embodiment B particularly to be emphasized are those in which
R1 is phenyl substituted in para or meta position by R3,
R2 is phenyl substituted by R12 and/or R13,
R3 is morpholin-4-ylmethyl, morpholin-4-ylethyl, morpholin-4-ylpropyl, 2-morpholin-4-ylethoxy, 3-morpholin-4-ylpropoxy, 4-methylpiperazin-1-ylmethyl, 4-methylpiperazin-1-ylethyl, 4-methylpiperazin-1-ylpropyl, 2-(4-methylpiperazin-1-yl)ethoxy, 3-(4-methylpiperazin-1-yl)propoxy, 1-methylpiperidin-4-ylmethoxy, 1-methylpiperidin-4-ylmethyl, 1-methylpiperidin-4-ylethyl, 1-methylpiperidin-4-ylpropyl, 2-(1-methylpiperidin-4-yl)ethoxy, 3-(1-methylpiperidin-4-yl)propoxy, pyrrolidin-1-ylpropyl, pyrrolidin-1-ylethyl, pyrrolidin-1-ylmethyl, 3-pyrrolidin-1-ylpropoxy, 2-pyrrolidin-1-ylethoxy, piperidin-1-ylmethyl, piperidin-1-ylethyl, piperidin-1-ylpropyl, 2-(piperidin-1-yl)ethoxy, 3-(piperidin-1-yl)propoxy,
R12 is fluorine, chlorine, cyano, methyl, isopropyl, trifluoromethyl or methoxy,
R13 is fluorine or chlorine,
   and the salts of these compounds.

Preferred compounds of formula 1 of embodiment B are those in which
R1 is phenyl substituted in para position by R3 or phenyl substituted in para position by R3 and in meta position by R4,
R2 is 2-fluorophenyl, 4-fluorophenyl, 2,6-difluorophenyl, 2,4-difluorophenyl, 2-fluoro-4-methylphenyl, 2-fluoro-4-methoxyphenyl, 3-fluoro-4-methoxyphenyl, 4-methylphenyl, 4-methoxyphenyl or 3-chloro-4-fluorophenyl,
R3 is 2-morpholin-4-ylethoxy, 3-morpholin-4-ylpropoxy, 4-methylpiperazin-1-ylethoxy, 4-methylpiperazin-1-ylpropoxy, morpholin-4-ylmethyl, morpholin-4-ylethyl, morpholin-4-ylpropyl, 1-methylpiperidin-4-ylmethoxy, 2-(1-methylpiperidin-4-yl)ethoxy, 4-methylpiperazin-1-ylethyl, 3-pyrrolidin-1-ylpropoxy, 2-pyrrolidin-1-ylethoxy or 3-pyrrolidin-1-ylpropyl,
R4 is fluorine,
   and the salts of these compounds.

Further preferred compounds of formula 1 of embodiment B are those in which
R1 is phenyl substituted in para or meta position by R3,
R2 is 2-fluorophenyl, 4-fluorophenyl, 2,6-difluorophenyl, 2,4-difluorophenyl, 4-methylphenyl, 4-methoxyphenyl or 3-chloro-4-fluorophenyl,
R3 is 2-morpholin-4-ylethoxy, 3-morpholin-4-ylpropoxy, 4-methylpiperazin-1-ylethoxy, 4-methylpiperazin-1-ylpropoxy, morpholin-4-ylmethyl, morpholin-4-ylethyl, morpholin-4-ylpropyl, 1-methylpiperidin-4-ylmethoxy, 2-(1-methylpiperidin-4-yl)ethoxy, 4-methylpiperazin-1-ylethyl, 3-pyrrolidin-1-ylpropoxy, 2-pyrrolidin-1-ylethoxy or 3-pyrrolidin-1-ylpropyl,
   and the salts of these compounds.

A further embodiment (embodiment C) of the compounds of formula 1 are those in which
R1 is aryl1,
   aryl1 substituted by R7 and/or R8,
   R9 or
   R10,
R2 is phenyl,
   phenyl substituted by R12 and/or R13,
   naphthalenyl,
   naphthalenyl substituted by R14 and/or R15,
   aryl2,
   aryl2 substituted by R16 and/or R17
   or a radical selected from

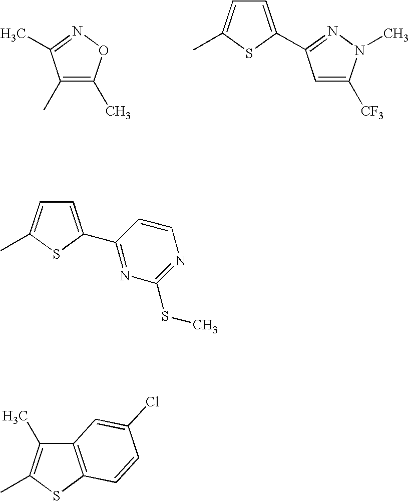

Aryl1 furanyl, thiophenyl, oxazolyl, isoxazolyl, thiazolyl, isothiazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl or dibenzofuranyl,
R7 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
   R31 is hydrogen, 1-4C-alkyl or 1-4C-alkoxy-2-4C-alkyl, and
   R32 is hydrogen, 1-4C-alkyl or 1-4C-alkoxy-2-4C-alkyl or wherein
   R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   n is an integer from 0 to 4,
   m is an integer from 2 to 4,
R8 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R9 is unsubstituted pyrrolyl, pyrazolyl, imidazolyl, indolyl, indazolyl, benzimidazolyl or benztriazolyl, or a radical selected from

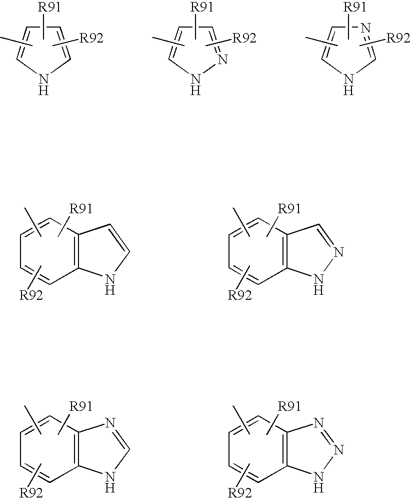

wherein
R91 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R92 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R10 is a radical selected from

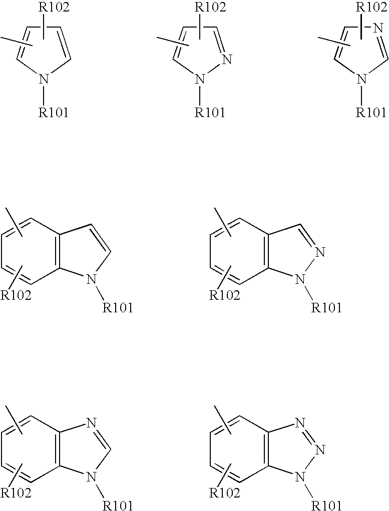

wherein
R101 is 1-4C-alkyl, 2,2,2-trifluoroethyl or 3,3,3-trifluoropropyl,
R102 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds with the proviso that the following compounds are excluded
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide.

Compounds of formula 1 of embodiment C to be emphasized are those in which
R1 is pyrid-3-yl, pyrid-4-yl, 2-methyl-2,3-dihydrobenzofuran-5-yl, benzo[1,3]dioxol-5-yl, 1-methyl-1H-pyrrol-3-yl, 4-methyl-thiophen-2-yl, 1-methyl-1H-pyrrol-2-yl, 1H-indol-5-yl, 1-methyl-1H-indol-3-yl, 1-methyl-1H-indol-5-yl, dibenzofuran-4-yl or 3,5-dimethyl-isoxazol-4-yl,
R2 is phenyl substituted by R12 and/or R13 or naphthalenyl,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds with the proviso that the following compounds are excluded
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide, and
4-Methyl-N-[4-(6-pyridin-3-yl-pyrimidin-4-ylamino)-phenyl]-benzenesulfonamide.

Compounds of formula 1 of embodiment C particularly to be emphasized are those in which
R1 is 2-methyl-2,3-dihydrobenzofuran-5-yl, benzo[1,3]dioxol-5-yl, 1-methyl-1H-pyrrol-3-yl, 4-methyl-thiophen-2-yl, 1-methyl-1H-pyrrol-2-yl, 1H-indol-5-yl, 1-methyl-1H-indol-3-yl or 1-methyl-1H-indol-5-yl.
R2 is phenyl substituted by R12 and/or R13 or naphthalenyl,
R12 is fluorine, chlorine, cyano, methyl, trifluoromethyl or methoxy,
R13 is fluorine, chlorine or methoxy,
   and the salts of these compounds with the proviso that the compound N-[4-(6-Benzo[1,3]dioxol-5-yl-pyrimidin-4-ylamino)-phenyl]-4-methyl-benzenesulfonamide is excluded.

Preferred compounds of formula 1 of embodiment C are those in which
either
R1 is 2-methyl-2,3-dihydrobenzofuran-5-yl, 1-methyl-1H-pyrrol-3-yl or 4-methylthiophen-2-yl, and
R2 is 2,6-difluorophenyl,
or
R1 is 1H-indol-5-yl, and
R2 is 2,6-difluorophenyl, 2,4-difluorophenyl, 2-fluoro-4-methylphenyl, 2-fluorophenyl or 3-fluorophenyl,
or
R1 is 1-methyl-1H-indol-5-yl or 1-methyl-1H-indol-3-yl, and
R2 is 2,6-difluorophenyl, 2-fluorophenyl or 4-methoxyphenyl,
or
R1 is 1-methyl-1H-indol-5-yl, and
R2 is 2-fluoro-4-methylphenyl,
   and the salts of these compounds.

Further preferred compounds of formula 1 of embodiment C are those in which
either
R1 is 2-methyl-2,3-dihydrobenzofuran-5-yl, 1-methyl-1H-pyrrol-3-yl or 4-methylthiophen-2-yl, and
R2 is 2,6-difluorophenyl,
or
R1 is 1H-indol-5-yl, and
R2 is 2,6-difluorophenyl, 2,4-difluorophenyl, 2-fluorophenyl or 3-fluorophenyl,
or
R1 is 1-methyl-1H-indol-5-yl or 1-methyl-1H-indol-3-yl, and
R2 is 2,6-difluorophenyl, 2-fluorophenyl or 4-methoxyphenyl,
   and the salts of these compounds.

Still a further embodiment (embodiment D) of the compounds of formula 1 are those in which
R1 is R11,
R2 is phenyl,
   phenyl substituted by R12 and/or R13,
   naphthalenyl,
   naphthalenyl substituted by R14 and/or R15,
   aryl2,
   aryl2 substituted by R16 and/or R17
   or a radical selected from

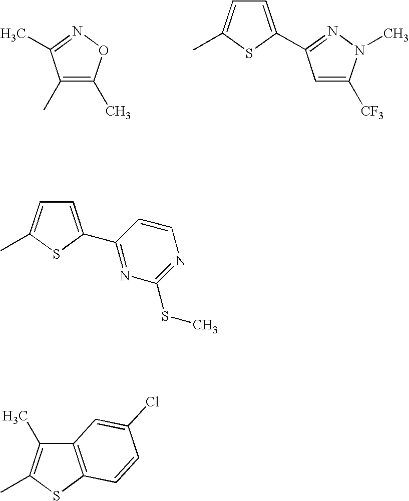

R11 is a radical selected from

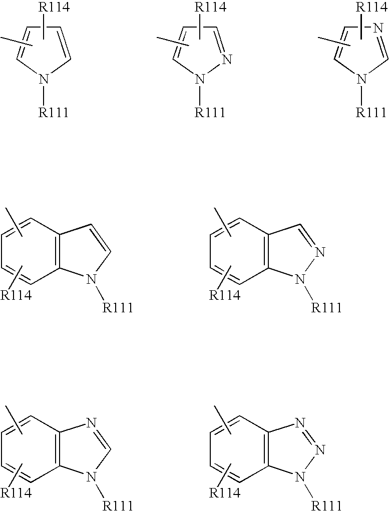

wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, —(CH₂)ₚ—N(R112)R113 or —CH₂CH(OH)CH₂N(R112)R113, wherein
   R112 is hydrogen, 1-4C-alkyl or 1-4C-alkoxy-2-4C-alkyl, and
   R113 is hydrogen, 1-4C-alkyl or 1-4C-alkoxy-2-4C-alkyl, or wherein
   R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Compounds of formula 1 of embodiment D to be emphasized are those in which
R1 is R11,
R2 is phenyl substituted by R12 and/or R13,
R11 is a radical selected from

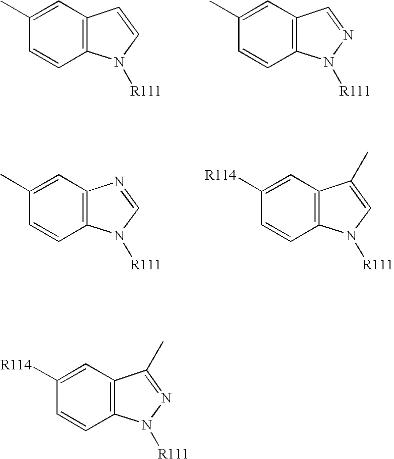

wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, —(CH₂)ₚ—N(R112)R113 or —CH₂CH(OH)CH₂N(R112)R113, wherein
   R112 is hydrogen, 1-4C-alkyl or 1-4C-alkoxy-2-4C-alkyl, and
   R113 is hydrogen, 1-4C-alkyl or 1-4C-alkoxy-2-4C-alkyl, or wherein
   R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
   p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Further compounds of formula 1 of embodiment D to be emphasized are those in which
R1 is R11,
R2 is phenyl substituted by R12 and/or R13,
R11 is a radical selected from

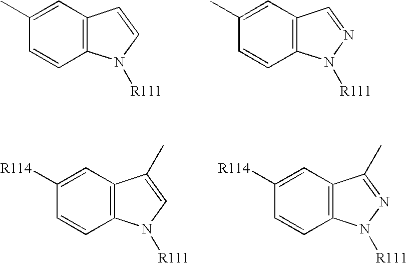

wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, —(CH₂)ₚ—N(R112)R113 or —CH₂CH(OH)CH₂N(R112)R113, wherein
   R112 is hydrogen, 1-4C-alkyl or 1-4C-alkoxy-2-4C-alkyl, and
   R113 is hydrogen, 1-4C-alkyl or 1-4C-alkoxy-2-4C-alkyl, or wherein
   R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan, 4-(1-4C-alkyl)-[1,4]diazepan-, morpholin-, thiomorpholin- or an azepan-ring,
   p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
   and the salts of these compounds.

Compounds of formula 1 of embodiment D particularly to be emphasized are those in which
R1 is R11,
R2 is phenyl substituted by R12 and/or R13,
R11 is a radical selected from

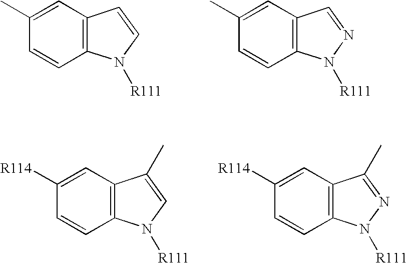

wherein
R111 is pyrrolidin-1-ylethyl, pyrrolidin-1-ylpropyl, piperidin-1-ylethyl, piperidin-1-ylpropyl, azepan-1-ylethyl, azepan-1-ylpropyl, (4-methyl-piperazin-1-yl)ethyl, (4-methyl-piperazin-1-yl)propyl, morpholin-4-ylethyl, morpholin-4-ylpropyl, (1-methyl-piperidin-4-yl)propyl, (1-methyl-piperidin-4-yl)ethyl, (1-methyl-piperidin-4-yl)methyl, dimethylaminoethyl, dimethylaminopropyl, dimethylaminobutyl, diethylaminoethyl, diethylaminopropyl or diethylaminobutyl,
R114 is hydrogen or fluorine,
R12 is fluorine, chlorine, cyano, methyl, isopropyl, trifluoromethyl or methoxy,
R13 is fluorine or chlorine,
   and the salts of these compounds.

Preferred compounds of formula 1 of embodiment D are those in which
R1 is R11,
R2 is 2-fluorophenyl, 4-fluorophenyl, 2,6-difluorophenyl, 2,4-difluorophenyl, 4-methylphenyl, 4-methoxy-phenyl, 3-fluoro-4-methoxyphenyl, 2-fluoro-4-methylphenyl, or 3-chloro-4-fluorophenyl,
R11 represents the following radical

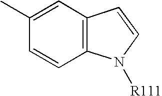

wherein
R111 is pyrrolidin-1-ylethyl, pyrrolidin-1-ylpropyl, (4-methyl-piperazin-1-yl)ethyl, (4-methyl-piperazin-1-yl)propyl, (1-methyl-piperidin-4-yl)ethyl, (1-methyl-piperidin-4-yl)methyl, dimethylaminoethyl, dimethylaminopropyl, diethylaminopropyl or diethylaminobutyl,
   and the salts of these compounds.

The compounds of formula 1 according to the invention can, for example, be prepared as described in reaction scheme 1.

The compounds of formula 1 according to the invention can be prepared, for example, starting from 3-oxo-propionic acid ester derivatives of formula 8, wherein R1 has the above-mentioned meanings and R stands, for example, for 1-4C-alkyl. In a first reaction step [step 1] the ester derivatives of formula 8 are reacted with thiourea to give the corresponding 2-mercapto-pyrimidin-4-ol derivatives of formula 7.

In the next two reaction steps [step 2, step 3] the mercapto group of the compounds of formula 7 is removed by treatment with Raney-Ni in ethanol (> compounds of formula 6) and the hydroxyl group is exchanged by a chlorine atom using POCl₃. The resulting 4-chloropyrimidine derivatives of formula 5 are then reacted with (4-aminophenyl)-carbamic acid tert butyl ester [step 4] to give [4-(pyrimidin-4-ylamino)phenyl]-carbamic acid tert butyl ester derivatives of formula 4.

Alternatively compounds of formula 4 can be obtained in a two steps procedure. In a first reaction step [step 5] 4,6-dichloropyrimidine is reacted with (4-aminophenyl)-carbamic acid tert butyl ester to give [4-(6-chloro-pyrimidin-4-yl-amino)-phenyl]-carbamic acid tert-butyl ester. Conversion of this compound with boronic acids of formula 9a [step 6] or boronic acid esters of formula 9b (for example 4,4,5,5-tetramethyl-(R1)-[1,3,2]dioxaborolane or 5,5-dimethyl-(R1)-[1,3,2]dioxaborinane), wherein R1 has the above-mentioned meanings lead to compounds of formula 4.

In the next reaction step [step 7] the amino group of the compounds of formula 4 is deprotected by HCl gas in dioxane leading to compounds of formula 3. In the final reaction step [step 8] the N-pyrimidin-4-yl-benzene-1,4-diamine derivatives of formula 3 are reacted with sulfonyl chloride derivatives of formula 2, wherein R2 has the above-mentioned meanings to yield the compounds of formula 1.

In addition, compounds of formula 1 can be obtained starting from [4-(6-chloro-pyrimidin-4-yl-amino)phenyl]-carbamic acid tert-butyl ester. The carbamic acid tert butyl ester can be deprotected by HCl gas in dioxane [step 9] to give N-(6-chloropyrimidin-4-yl)-benzene-1,4-diamine after basic workup, which on its part is reacted with sulfonyl chloride derivatives of formula 2 [step 10], wherein R2 have the above-mentioned meanings. The resulting compounds of formula 10 are reacted in a final reaction step [step 11] with boronic acids of formula 9a or boronic acid esters of formula 9b (for example 4,4,5,5-tetramethyl-(R1)-[1,3,2]dioxaborolane or 5,5-dimethyl-(R1)-[1,3,2]dioxaborinane), wherein R1 has the above-mentioned meanings to yield the compounds of formula 1.

Further information for the preparation of boronic acid esters can be found, for example, in Murata et al., J Org Chem 2000, 65, 6458 or J Org Chem 1997, 62, 164.

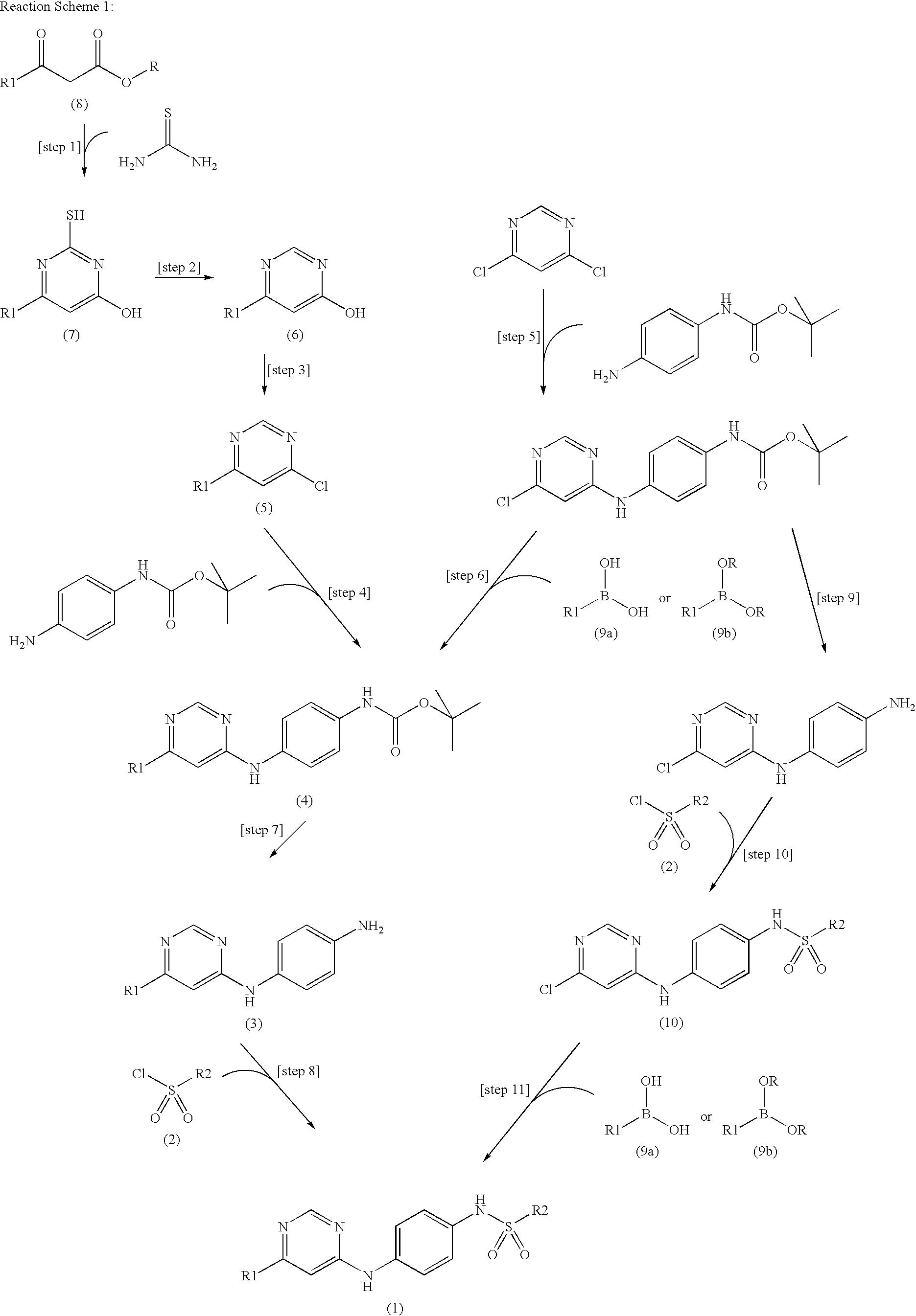

Suitably, the conversions are carried out analogous to methods, which are familiar per se to the person skilled in the art, for example, in the manner which is described in the following examples.

It is known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1991.

The substances according to the invention are isolated and purified in a manner known per se, e.g. by distilling off the solvent in vacuo and recrystallizing the residue obtained from a suitable solvent or subjecting it to one of the customary purification methods, such as column chromatography on a suitable support material.

Salts are obtained by dissolving the free compound in a suitable solvent (for example a ketone like acetone, methylethylketone, or methylisobutylketone, an ether, like diethyl ether, tetrahydrofuran or dioxane, a chlorinated hydrocarbon, such as methylene chloride or chloroform, or a low molecular weight aliphatic alcohol, such as ethanol, isopropanol) which contains the desired acid, or to which the desired acid is then added. The salts are obtained by filtering, reprecipitating, precipitating with a non-solvent for the addition salt or by evaporating the solvent Salts obtained can be converted by basification into the free compounds which, in turn, can be converted into salts. In this manner, pharmacologically non-tolerable salts can be converted into pharmacologically tolerable salts.

The following examples illustrate the invention in greater detail, without restricting it. As well, further compounds of formula 1, of which the preparation is explicitly not described, can be prepared in an analogous way or in a way which is known by a person skilled in the art using customary preparation methods.

In the examples, h stands for hour(s), calc for calculated, fnd for found and MS for mass spectroscopy. ¹H-NMR stands for proton nuclear magnetic resonance spectroscopy. The compounds, which are mentioned in the examples as well as their salts are preferred compounds of the invention.

Several of the examples are prepared as formic acid salts; in these cases the calculated total weight and the total molecular formula corresponds to the addition of the calculated total weight respectively total molecular formula of the indicated compound and the formic acid.

### EXAMPLES

### Final Products

### 1. 2,6-Difluoro-N-{4-[6-(4-fluoro-phenyl)pyrimidin-4-ylamino]-phenyl}-benzenesulfonamide

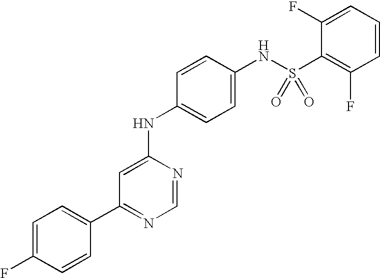

N-[6-(4-Fluoro-phenyl)-pyrimidin-4-yl]-benzene-1,4-diamine (compound A9, 280 mg) and 2,6-lutidine (150 μL) is dissolved in a mixture of dioxane (10 ml), dimethylformamide (0.5 ml) and water (0.1 ml). To the well stirred mixture a solution of 2,6-Difluoro-benzenesulfonyl chloride (213 mg) in dioxane (5 ml) is added and stirring is continued for about 2 h at ambient temperature. Saturated aqueous NaCl solution (5 ml) is added, the organic layer is separated and filtered through a plug of neutral alumina. The filtrate is concentrated in vacuo. The residue is dissolved in ethyl acetate and again filtered through neutral alumina. The filtrate is concentrated in vacuo. After crystallization from ethyl acetate and tert-BuOMe 148 mg of pure product is obtained as off-white solid.

¹H NMR (DMSC-d₆) [symbol] (ppm): 10.67 (s, 1H, —NH); 9.62 (s, 1H, —NH); 8.65 (s, 1H); 8.06 (d×d, J₁=8.9 Hz, J2=5.5 Hz, 2H); 7.69 (m, 1H); 7.60 (d, J=8.9 Hz, 2H); 7.31 (m, 2H+2H); 7.14 (s, 1H); 7.12 (d, J=8.8 Hz, 2H).

MS (ESI): calc: C₂₂H₁₅F₃N₄O₂S (456.45) fnd: [MH⁺] 457.1 [m/e=457.1 (MH⁺, 100%)].

### 2. 2,4-Difluoro-N-(4-{6-[4-(1-methyl-piperidin-4-ylmethoxy)-phenyl]-pyrimidin-4-ylamino}-phenyl)-benzenesulfonamide

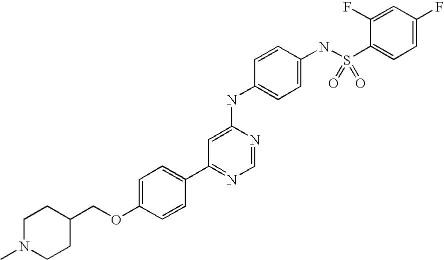

A mixture of N-[4-(6-chloro-pyrimidin-4-ylamino)-phenyl]-2,4-difluoro-benzenesulfonamide (compound A11, 455 mg), 1-methyl-4-[4-(4,4,5,5-tetramethyl-[1,3,2]dioxaborolan-2-yl)-phenoxymethyl]-piperidine (compound A14, 331 mg), 2M aqueous Cs₂CO₃ (1.2 ml) and trans-dichlorobis(tricyclohexylphosphine)-palladium (20 mg) in degassed dimethoxyethane and ethanol is heated in a microwave oven (Emrys Optimizer from Personal Chemistry) at 140° C. in a sealed vial for 20 min. The reaction mixture is filtered through a plug of Extrelute®. The filtrate is concentrated in vacuo. The crude product is purified by preparative HPLC (gradient from 25% to 75% acetonitrile in water buffered with formic acid and ammonium formic acid salt). The combined product fractions are freeze dried to obtain the pure product as formic acid salt. The free base is precipitated from an aqueous solution by careful addition of aqueous ammonia. Crystallization from acetonitrile and water affords 372 mg of pure compound as colourless solid.

1H-NMR (DMSO-d₆) [symbol] (ppm): 9.49 (s, 1H, —NH); 8.60 (s, 1H); 7.95 (d, J=8.9 Hz, 2H); 7.68 (m, 1H); 7.56 (d, J=8.9 Hz, 2H); 7.24 (d×d J₁=J₂=9.0 Hz, 2H); 7.09 (d, J=5.9 Hz, 2H); 7.08 (s, 1H); 7.05 (d, J=5.9 Hz, 2H); 3.90 (d, J=5.8 Hz, 2H); 2.82 (˜d, J˜11.1 Hz, 2H); 2.19 (s, 3H); 1.92 (˜t, J˜11.5 Hz, 2H); 1.75 (˜m, 2H+1H); 1.43 (˜m, 2H).

MS (ESI): calc: C₂₉H₂₉F₂N₅O₃S (565.65) fnd: [MH⁺] 566.3 [m/e=566.3 (MH⁺, 100%)]

The following compounds can be prepared analogously to the methods described above:

### 3. 5-(1-Methyl-5-trifluoromethyl-1H-pyrazol-3-yl)-thiophene-2-sulfonic acid [4-(6-phenylpyrimidin-4-ylamino)-phenyl]-amide

MS: calc: C₂₅H₁₉F₃N₆O₂S₂ (556.59) fnd: [MH⁺] 557.2

### 4. 4-Cyano-N-[4-(6-phenyl-pyrimidin-4-ylamino)phenyl]-benzenesulfonamide

MS: C₂₃H₁₇N₅O₂S (427.49) fnd: [MH⁺] 428.2

### 5. 4-Isopropyl-N-[4-(6-phenyl-pyrimidin-4-ylamino)-phenyl]-benzenesulfonamide

MS: calc: C₂₅H₂₄N₄O₂S (444.56) fnd: [MH⁺] 445.2

### 6. 4-Methoxy-N-[4-(6-phenyl-pyrimidin-4-ylamino)-phenyl]-benzenesulfonamide

MS: calc: C₂₃H₂₀N₄O₃S (432.50) fnd: [MH⁺] 433.2

### 7. 2-Fluoro-N-[4-(6-phenyl-pyrimidin-4-ylamino)phenyl]-benzenesulfonamide

MS: cal: C₂₂H₁₇FN₄O₂S (420.47) fnd: [MH⁺] 421.2

### 8. 2,6-Difluoro-N-[4-(6-phenyl-pyrimidin-4-ylamino)-phenyl]-benzenesulfonamide

MS: calc: C₂₂H₁₈F₂N₄O₂S (438.46) fnd: [MH⁺] 439.1

### 9. 2,4-Difluoro-N-[4-(6-naphthalen-1-yl-pyrimidin-4-ylamino)-phenyl]-benzenesulfonamide

MS: calc: C₂₆H₁₈F₂N₄O₂S (488.52) fnd: [MH⁺] 489.2

### 10. 3,4-Dichloro-N-[4-(6-naphthalen-1-yl-pyrimidin-4-ylamino)-phenyl]-benzenesulfonamide

MS: calc: C₂₆H₁₈Cl₂N₄O₂S (521.43) fnd: [MH⁺] 521.1

### 11. 3-Methyl-N-[4-(6-naphthalen-1-yl-pyrimidin-4-ylamino)-phenyl]-benzenesulfonamide

MS: calc C₂₇H₂₂N₄O₂S (466.57) fnd: [MH⁺] 467.1

### 12. N-[4-(6-Naphthalen-1-yl-pyrimidin-4-ylamino)-phenyl]-trifluoromethyl-benzenesulfonamide

MS: calc: C₂₇H₁₉F₃N₄O₂S (520.54) fnd: [MH⁺] 521.1

### 13. 2-Fluoro-N-[4-(6-naphthalen-1-yl-pyrimidin-4-ylamino)-phenyl]-benzenesulfonamide

MS: calc: C₂₆H₁₉FN₄O₂S (470.53) fnd: [MH⁺] 471.2

### 14. 3-Bromo-N-[4-(6-naphthalen-1-yl-pyrimidin-4-ylamino)-phenyl]-benzenesulfonamide

MS: calc: C₂₆H₁₉BrN₄O₂S (531.43) fnd: [MH⁺] 533.0

### 15. 3-Chloro-4-methyl-N-[4-(6-naphthalen-1-yl-pyrimidin-4-ylamino)-phenyl]-benzenesulfonamide

MS: calc: C₂₇H₂₁ClN₄O₂S (501.01) fnd: [MH⁺] 501.1

### 16. 2,6-Difluoro-N-[4-(6-naphthalen-1-yl-pyrimidin-4-ylamino)-phenyl]-benzenesulfonamide

MS: calc: C₂₆H₁₈F₂N₄O₂S (488.52) fnd: [MH⁺] 489.1

### 17. N-{4-[6-(3-Methoxy-phenyl)-pyrimidin-4-ylamino]-phenyl}-trifluoromethyl-benzenesulfonamide

MS: calc: C₂₄H₁₉F₃N₄O₃S (500.50) fnd: [MH⁺] 501.2

### 18. 2,4-Difluoro-N-{4-[6-(3-methoxy-phenyl)-pyrimidin-4-ylamino]-phenyl}-benzenesulfonamide

MS: calc: C₂₃H₁₈F₂N₄O₃S (468.49) fnd: [MH⁺] 469.1

### 19. 4-Cyano-N-{4-[6-(3-methoxy-phenyl)-pyrimidin-4-ylamino]phenyl}benzenesulfonamide

MS: calc: C₂₄H₁₉N₅O₃S (457.51) fnd: [MH⁺] 458.1

### 20. 2-Fluoro-N-{4-[6-(3-methoxy-phenyl)-pyrimidin-4-ylamino]-phenyl}-benzenesulfonamide

MS: calc: C₂₃H₁₉FN₄O₃S (450.50) fnd: [MH⁺] 451.2

### 21. 2,4-Difluoro-N-{4-[6-(4-fluoro-phenyl)-pyrimidin-4-ylamino]-phenyl}-benzenesulfonamide

MS: calc: C₂₂H₁₅F₃N₄O₂S (456.45) fnd: [MH⁺] 457.1

### 22. 4-Cyano-cyclohexa-1,5-dienesulfonic acid {4-[6-(4-fluoro-phenyl)-pyrimidin-4-ylamino]-phenyl}-amide

MS: calc: C₂₃H₁₈FN₅O₂S (447.49) fnd: [MH⁺] 446.1

### 23. 2-Fluoro-N-{4-[6-(4-fluoro-phenyl)-pyrimidin-4-ylamino]-phenyl}-benzenesulfonamide

MS: calc: C₂₂H₁₆F₂N₄O₂S (438.46) fnd: [MH⁺] 439.1

### 24. 2,4-Difluoro-N-{4-[6-(3-fluoro-phenyl)-pyrimidin-4-ylamino]-phenyl}-benzenesulfonamide

MS: calc: C₂₂H₁₅F₃N₄O₂S (456.45) fnd: [MH⁺] 457.2

### 25. N-{4-[6-(4-Acetyl-phenyl)-pyrimidin-4-ylamino]-phenyl}-2,4-difluoro-benzenesulfonamide

MS: calc: C₂₄H₁₈F₂N₄O₃S (480.50) fnd: [MH⁺] 481.3

### 26. 2,6-Difluoro-N-{4-[6-(3-fluoro-phenyl)-pyrimidin-4-ylamino]-phenyl}-2,4-difluoro-benzenesulfonamide

MS: calc: C₂₂H₁₅F₃N₄O₂S (456.45) fnd: [MH⁺] 457.2

### 27. N-{4-[6-(4-Acetyl-phenyl)-pyrimidin-4-ylamino]-phenyl}-2,6-difluoro-benzenesulfonamide

MS: calc: C₂₄H₁₈F₂N₄O₃S (480.50) fnd: [MH⁺] 481.2

### 28. N-{4-[6-(4-Acetyl-phenyl)-pyrimidin-4-ylamino]-phenyl}-3-chloro-4-fluoro-benzenesulfonamide

MS: calc: C₂₄H₁₈ClFN₄O₃S (496.95) fnd: [MH⁺] 497.2

### 29. 2-Fluoro-N-{4-[6-(4-methoxy-phenyl)-pyrimidin-4-ylamino]-phenyl}-benzenesulfonamide

MS: calc: C₂₃H₁₉FN₄O₃S (450.50) fnd: [MH⁺] 451.2

### 30. N-{4-[6-(4-Acetyl-phenyl)-pyrimidin-4-ylamino]-phenyl}-3,4-difluoro-benzenesulfonamide

MS: calc: C₂₄H₁₈F₂N₄O₃S (480.50) fnd: [MH⁺] 481.2

### 31. N-{4-[6-(4-Acetyl-phenyl)-pyrimidin-4-ylamino]-phenyl}-3-fluoro-benzenesulfonamide

MS: calc: C₂₄H₁₉FN₄O₃S (462.51) fnd: [MH⁺] 463.2

### 32. 3-Fluoro-N-{4-[6-(4-methoxy-phenyl)-pyrimidin-4-ylamino]-phenyl}-benzenesulfonamide

MS: calc: C₂₃H₁₉FN₄O₃S (450.50) fnd: [MH⁺] 451.2

### 33. N-{4-[6-(4-Acetyl-phenyl)-pyrimidin-4-ylamino]-phenyl}-3-methyl-benzenesulfonamide

MS: calc: C₂₅H₂₂N₄O₃S (458.54) fnd: [MH⁺] 459.2

### 34. N-{4-[6-(3-Fluoro-phenyl)-pyrimidin-4-ylamino]-phenyl}-methyl-benzenesulfonamide

MS: calc: C₂₃H₁₉FN₄O₂S (434.50) fnd: [MH⁺] 435.2

### 35. N-{4-[6-(4-Acetyl-phenyl)-pyrimidin-4-ylamino]-phenyl}-4-methyl-benzenesulfonamide

MS: calc: C₂₅H₂₂N₄O₃S (458.54) fnd: [MH⁺] 459.3

### 36. 2,6-Difluoro-N-{4-[6-(4-methoxy-phenyl)-pyrimidin-4-ylamino]-phenyl}-benzenesulfonamide

MS: calc: C₂₃H₁₈F₂N₄O₃S (468.49) fnd: [MH⁺] 469.2

### 37. 2,6-Difluoro-N-(4-{6-[4-(2-morpholin-4-yl-ethoxy)-phenyl]-pyrimidin-4-ylamino}-phenyl)-benzenesulfonamide

MS: calc: C₂₈H₂₇F₂N₅O₄S (567.62) fnd: [MH⁺] 568.3

### 38. 2,6-Difluoro-N-(4-{6-[4-(3-morpholin-4-yl-propoxy)-phenyl]-pyrimidin-4-ylamino}-phenyl)-benzenesulfonamide

MS: calc: C₂₉H₂₉F₂N₅O₄S (567.62) fnd: [MH⁺] 582.3

### 39. 2,6-Difluoro-N-(4-{6-[3-(3-morpholin-4-yl-propoxy)-phenyl]-pyrimidin-4-ylamino}-phenyl)-benzenesulfonamide

MS: calc: C₂₉H₂₉F₂N₅O₄S (581.65) fnd: [MH⁺] 582.3

### 40. 2-Fluoro-N-(4-{6-[4-(2-morpholin-4-yl-ethoxy)-phenyl]-pyrimidin-4-ylamino}-phenyl)-benzenesulfonamide

MS: calc: C₂₈H₂₈FN₅O₄S (549.63) fnd: [MH⁺] 550.4

### 41. 2-Fluoro-N-(4-{6-[4-(3-morpholin-4-yl-propoxy)-phenyl]-pyrimidin-4-ylamino}-phenyl)-benzenesulfonamide

MS: calc: C₂₉H₃₀FN₅O₄S (563.66) fnd: [MH⁺] 564.4

### 42. 2-Fluoro-N-(4-{6-[3-(2-morpholin-4-yl-ethoxy)-phenyl]-pyrimidin-4-ylamino}-phenyl)-benzenesulfonamide

MS: calc: C₂₈H₂₈FN₅O₄S (549.93) fnd: [MH⁺] 550.4

### 43. 2-Fluoro-N-(4-{6-[3-(3-morpholin-4-yl-propoxy)-phenyl]-pyrimidin-4-ylamino}-phenyl)-benzenesulfonamide

MS: calc: C₂₉H₃₀FN₅O₄S (563.66) fnd: [MH⁺] 564.4

### 44. 4-Methoxy-N-(4-{6-[4-(2-morpholin-4-yl-ethoxy)-phenyl]-pyrimidin-4-ylamino}-phenyl)-benzenesulfonamide

MS: calc: C₂₉H₃₁N₅O₅S (561.66) fnd: [MH⁺] 562.4

### 45. 4-Methoxy-N-(4-{6-[4-(3-morpholin-4-yl-propoxy)-phenyl]-pyrimidin-4-ylamino}-phenyl)-benzenesulfonamide

MS: calc: C₃₀H₃₃N₅O₅S (575.69) fnd: [MH⁺] 576.4

### 46. 4-Methoxy-N-(4-{6-[3-(3-morpholin-4-yl-propoxy)-phenyl]-pyrimidin-4-ylamino}-phenyl)-benzenesulfonamide

MS: calc: C₃₀H₃₃N₅O₅S (575.69) fnd: [MH⁺] 576.4

### 47. 2,6-Difluoro-N-[4-(6-{4-[2-(4-methyl-piperazin-1-yl)-ethoxy]-phenyl}-pyrimidin-4-ylamino)-phenyl]-benzenesulfonamide

MS: calc: C₂₉H₃₀F₂N₆O₃S (580.66) fnd: [MH⁺] 581.4

### 48. 2,6-Difluoro-N-[4-(6-{3-[2-(4-methyl-piperazin-1-yl)-ethoxy]-phenyl}-pyrimidin-4-ylamino)-phenyl]-benzenesulfonamide

MS: calc: C₂₉H₃₀F₂N₆O₃S (580.66) fnd: [MH⁺] 581.4

### 49. 2,6-Difluoro-N-[4-(6-{3-[2-(4-methyl-piperazin-1-yl)-ethoxy]-phenyl}-pyrimidin-4-ylamino)-phenyl]-benzenesulfonamide

MS: calc: C₃₀H₃₂F₂N₈O₃S (594.69) fnd: [MH⁺] 595.4

### 50. 2-Fluoro-N-[4-(6-{4-[2-(4-methyl-piperazin-1-yl)-ethoxy]-phenyl}-pyrimidin-4-ylamino)-phenyl]-benzenesulfonamide

MS: calc: C₂₉H₃₁FN₆O₃S (562.67) fnd: [MH⁺] 563.3

### 51. 2-Fluoro-N-[4-(6-{3-[3-(4-methyl-piperazin-1-yl)-propoxy]-phenyl}-pyrimidin-4-ylamino)-phenyl]-benzenesulfonamide

MS: calc: C₃₀H₃₃FN₆O₃S (576.70) fnd: [MH⁺] 577.4

### 52. 4-Methoxy-N-[4-(6-{4-[2-(4-methyl-piperazin-1-yl)-ethoxy]-phenyl}-pyrimidin-4-ylamino)-phenyl]-benzenesulfonamide

MS: calc: C₂₀H₃₄N₆O₄S (574.71) fnd: [MH⁺] 575.4

### 53. 2,4-Difluoro-N-[4-(6-{4-[2-(4-methyl-piperazin-1-yl)-ethoxy]-phenyl}-pyrimidin-4-ylamino)-phenyl]-benzenesulfonamide

MS: calc: C₂₉H₃₀F₂N₆O₃S (580.66) fnd: [MH⁺] 581.4

### 54. 4-Methyl-N-[4-(6-{4-[2-(4-methyl-piperazin-1-yl)-ethoxy]-phenyl}-pyrimidin-4-ylamino)-phenyl]-benzenesulfonamide

MS: calc: C₃₀H₃₄N₈O₃S (558.71) fnd: [MH⁺] 595.4

### 55. 4-Methyl-N-[4-(6-{4-[3-(4-methyl-piperazin-1-yl)-propoxy]-phenyl}-pyrimidin-4-ylamino)-phenyl]-benzenesulfonamide

MS: calc: C₃₁H₃₆N₆O₃S (572.73) fnd: [MH⁺] 573.4

### 56. 3-Chloro-4-fluoro-N-[4-(6-{4-[2-(4-methyl-piperazin-1-yl)-ethoxy]-phenyl}-pyrimidin-4-ylamino)-phenyl]-benzenesulfonamide

MS: calc: C₂₉H₃₀ClFN₆O₃S (597.12) fnd: [MH⁺] 597.4

### 57. 3-Chloro-4-fluoro-N-[4-(6-{4-[3-(4-methyl-piperazin-1-yl)-propoxy]-phenyl}-pyrimidin-4-ylamino)-phenyl]-benzenesulfonamide

MS: calc: C₃₀H₃₂ClFN₆O₃S (611.14) fnd: [MH⁺] 611.4

### 58. 2,4-Difluoro-N-(4-{6-[4-(3-morpholin-4-yl-propoxy)-phenyl]-pyrimidin-4-ylamino}-phenyl)-benzenesulfonamide

MS: calc: C₂₉H₂₉F₂N₅O₄S (581.65) fnd: [M+NH₄⁺] 598.5

### 59. 2,4-Difluoro-N-(4-{6-[3-(2-morpholin-4-yl-ethoxy)-phenyl]-pyrimidin-4-ylamino}-phenyl)-benzenesulfonamide

MS: calc: C₂₉H₂₇F₂N₅O₄S (567.62) fnd: [M+NH₄⁺] 584.4

### 60. 4-Methyl-N-(4-{6-[4-(3-morpholin-4-yl-propoxy)-phenyl]-pyrimidin-4-ylamino}phenyl)-benzenesulfonamide

MS: calc: C₃₀H₃₃N₅O₄S (559.69) fnd: [MH⁺] 560.4

### 61. 4-Methyl-N-(4-{6-[3-(2-morpholin-4-yl-ethoxy)-phenyl]-pyrimidin-4-ylamino}-phenyl)-benzenesulfonamide

MS: calc: C₂₉H₃₁N₅O₄S (545.67) fnd: [MH⁺] 546.4

### 62. 3-Chloro-4-fluoro-N-(4-{6-[4-(2-morpholin-4-yl-ethoxy)-phenyl]-pyrimidin-4-ylamino}-phenyl)-benzenesulfonamide

MS: calc: C₂₈H₂₇ClFN₅O₄S (584.07) fnd: [MH⁺] 584.5

### 63. 3-Chloro-4-fluoro-N-(4-{6-[4-(3-morpholin-4-yl-propoxy)-phenyl]-pyrimidin-4-ylamino}-phenyl)-benzenesulfonamide

MS: calc: C₂₉H₂₉ClFN₅O₄S (598.10) fnd: [MH⁺] 598.5

### 64. 2-Fluoro-N-{4-[6-(4-morpholin-4-ylmethyl-phenyl)-pyrimidin-4-ylamino]-phenyl}-benzenesulfonamide formic acid salt

MS: calc: C₂₈H₂₈FN₅O₅S (565.63) fnd: [MH⁺] 520.2

### 65. 2-Fluoro-N-[4-(6-{4-[2-(4-methyl-piperazin-1-yl)-ethoxy]-phenyl}-pyrimidin-4-ylamino)-phenyl]-benzenesulfonamide formic acid salt

MS: calc: C₂₀H₃₃FN₅O₅S (608.70) fnd: [MH⁺] 5635

### 66. 2-Fluoro-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₁H₃₄FN₅O₅S (607.71) fnd: [MH⁺] 562.2

### 67. 2-Fluoro-N-(4-{6-[4-(1-methyl-piperidin-4-ylmethoxy)-phenyl]-pyrimidin-4-ylamino}-phenyl)-benzenesulfonamide formic acid salt

MS: calc: C₃₀H₃₂FN₅O₅S (593.68) fnd: [MH⁺] 548.1

### 68. 2-Fluoro-N-(4-{6-[4-(2-morpholin-4-yl-ethyl)-phenyl]-pyrimidin-4-ylamino}-phenyl)-benzenesulfonamide formic acid salt

MS: calc: C₂₉H₃₀FN₅O₅S (579.66) fnd: [MH⁺] 534.3

### 69. 2-Fluoro-N-(4-{6-[4-(3-morpholin-4-yl-propyl)-phenyl]-pyrimidin-4-ylamino}-phenyl)-benzenesulfonamide formic acid salt

MS: calc: C₃₀H₃₂FN₅O₅S (593.68) fnd: [MH⁺] 548.2

### 70. 2-Fluoro-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₁H₃₄FN₅O₅S (607.71) fnd: [MH⁺] 562.3

### 71. 2,6-Difluoro-N-(4-{6-[4-(3-pyrrolidin-1-yl-propoxy)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.1

### 72. 2,6-Difluoro-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₁H₃₃F₂N₅O₅S (625.70) fnd: [MH⁺] 580.2

### 73. 2,6-Difluoro-N-(4-{6-[4-(1-methyl-piperidin-4-ylmethoxy)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.1

### 74. 2,6-Difluoro-N-(4-{6-[4-(3-morpholin-4-yl-propyl)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.3

### 75. 2,6-Difluoro-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₁H₃₃F₂N₅O₅S (625.70) fnd: [MH⁺] 580.2

### 76. 4-Methoxy-N-(4-{6-[4-(3-pyrrolidin-1-yl-propoxy)-phenyl]-pyrimidin-4-ylamino}-phenyl)-benzenesulfonamide formic acid salt

MS: calc: C₃₁H₃₅N₅O₅S (605.72) fnd: [MH⁺] 560.3

### 77. 4-Methoxy-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₂H₃₇N₅O₅S (619.75) fnd: [MH⁺] 574.3

### 78. 4-Methyl-N-(4-[6-{4-(3-pyrrolidin-1-yl-propoxy)-phenyl]-pyrimidin-4-ylamino}-phenyl)-benzenesulfonamide formic acid salt

MS: calc: C₃₁H₃₅N₅O₅S (589.72) fnd: [MH⁺] 544.3

### 79. 4-Methyl-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₂H₃₇N₅O₅S (603.75) fnd: [MH⁺] 558.2

### 80. 4-Methyl-N-(4-{6-[4-(2-morpholin-4-yl-ethyl)-phenyl]-pyrimidin-4-ylamino}-phenyl)-benzenesulfonamide formic acid salt

MS: calc: C₃₀H₃₃N₅O₅S (575.69) fnd: [MH⁺] 530.3

### 81. 4-Methyl-N-(4-{6-[4-(3-morpholin-4-yl-propyl)-phenyl]pyrimidin-4-ylamino}-phenyl)-benzenesulfonamide formic acid salt

MS: calc: C₃₁H₂₅N₅O₅S (589.72) fnd: [MH⁺] 544.3

### 82. 4-Methyl-N-(4-{6-[4-(2-pyrrolidin-1-yl-ethoxy)-phenyl]-pyrimidin-4-ylamino}-phenyl)-benzenesulfonamide formic acid salt

MS: calc: C₃₃H₃₃N₅O₅S (575.69) fnd: [MH⁺] 530.3

### 83. 4-Methyl-N-(4-{6-[4-(1-methyl-piperidin-4-ylmethoxy)-phenyl]-pyrimidin-4-ylamino}-phenyl)-benzenesulfonamide formic acid salt

MS: calc: C₃₁H₃₅N₅O₅S (589.72) fnd: [MH⁺] 544.3

### 84. 4-Methyl-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₂H₃₇N₅O₉S (603.75) fnd: [MH⁺] 558.3

### 85. 4-Methyl-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₃H₃₅N₅O₄S (612.76) fnd: [MH⁺] 567.3

### 86. 4-Methyl-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₃H₃₇N₇O₄S (627.77) fnd: [MH⁺] 582.4

### 87. 2,4-Difluoro-N-(4-{6-[4-(3-pyrrolidin-1-yl-propoxy)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.2

### 88. 2,4-Difluoro-N-{4-[6-(4-morpholin-4-ylmethyl-phenyl)-pyrimidin-4-ylamino]-phenyl}-benzenesulfonamide formic acid salt

MS: calc: C₂₈H₂₇F₂N₅O₅S (583.62) fnd: [MH⁺] 538.1

### 89. 2,4-Difluoro-N-[4-(6-{4-[2-(4-methyl-piperazin-1-yl)-ethoxy]-phenyl}-pyrimidin-4-ylamino)-phenyl]-benzenesulfonamide formic acid salt

MS: calc: C₃₀H₃₂F₂N₅O₅S (626.69) fnd: [MH⁺] 581.2

### 90. 2,4-Difluoro-N-[4-(6-{4-[2-(1-methyl-piperidin-4-yl)-ethoxy]-phenyl}-pyrimidin-4-ylamino)-phenyl]-benzenesulfonamide formic acid salt

MS: calc: C₃₀H₃₂F₂N₅O₅S (625.70) fnd: [MH⁺] 580.2

### 91. 2,4-Difluoro-N-(4-{6-[4-(1-methyl-piperidin-4-ylmethoxy)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.2

### 92. 2,4-Difluoro-N-(4-{6-[4-(2-morpholin-4-yl-ethyl)-phenyl]-pyrimidin-4-ylamino}-phenyl)-benzenesulfonamide formic acid salt

MS: calc: C₂₉H₂₉F₂N₅O₅S (597.65) fnd: [MH⁺] 552.2

### 93. 2,4-Difluoro-N-(4-{6-[4-(3-morpholin-4-yl-propyl)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.3

### 94. 2,4-Difluoro-N-(4-{6-[4-(2-pyrrolidin-1-yl-ethoxy)-phenyl]-pyrimidin-4-ylamino}-phenyl)-benzenesulfonamide formic acid salt

MS: calc: C₂₉H₂₉F₂N₅O₅S (597.65) fnd: [MH⁺] 552.2

### 95. 3-Chloro-4-fluoro-N-[4-(6-{4-[2-(4-methyl-piperazin-1-yl)-ethoxy]phenyl}-pyrimidin-4-ylamino)-phenyl]-benzenesulfonamide formic acid salt

MS: calc: C₃₀H₃₂ClFN₆O₆S (643.14) fnd: [MH⁺] 597.3

### 96. 3-Chloro-4-fluoro-N-(4-{6-[4-(3-morpholin-4-yl-propyl)-phenyl]-pyrimidin-4-ylamino}-phenyl)-benzenesulfonamide formic acid salt

MS: calc: C₃₀H₃₁ClFN₅O₅S (628.13) fnd: [MH⁺] 582.3

### 97. 3-Chloro-4-fluoro-N-(4-{6-[4-(2-pyrrolidin-1-yl-ethoxy)-phenyl]-pyrimidin-4-ylamino}-phenyl)-benzenesulfonamide formic acid salt

MS: calc: C₂₉H₂₉ClFN₅O₅S (614.10) fnd: [MH⁺] 568.3

### 98. 2-Fluoro-N-(4-{6-[4-(2-pyrrolidin-1-yl-ethoxy)-phenyl]-pyrimidin-4-ylamino}-phenyl)-benzenesulfonamide formic acid salt

MS: calc: C₂₉H₃₀FN₅O₅S (579.66) fnd: [MH⁺] 534.2

### 99. 2-Fluoro-N-(4-{6-[4-(3-pyrrolidin-1-yl-propoxy)-phenyl]-pyrimidin-4-ylamino}-phenyl)-benzenesulfonamide formic acid salt

MS: calc: C₃₀H₃₂FN₅O₅S (593.68) fnd: [MH⁺] 5485

### 100. 2,6-Difluoro-N-[4-(6-{4-[2-(4-methyl-piperazin-1-yl)-ethoxy]-phenyl}-pyrimidin-4-ylamino)-phenyl]-benzenesulfonamide formic acid salt

MS: calc: C₃₀H₃₂F₂N₆O₅S (626.69) fnd: [MH⁺] 5812

### 101. 2,6-Difluoro-N-(4-{6-[4-(2-pyrrolidin-1-yl-ethoxy)-phenyl]-pyrimidin-4-ylamino}-phenyl)-benzenesulfonamide formic acid salt

MS: calc: C₂₉H₂₉F₂N₅O₅S (597.65) fnd: [MH⁺] 5525

### 102. 4-Methoxy-N-[4-(6-{4-[2-(4-methyl-piperazin-1-yl)-ethoxy]-phenyl}-pyrimidin-4-ylamino)-phenyl]-benzenesulfonamide formic acid salt

MS: calc: C₃₁H₃₆N₆O₆S (620.73) fnd: [MH⁺] 575.3

### 103. 4-Methoxy-N-(4-{6-[4-(1-methyl-piperidin-4-ylmethoxy)-phenyl]-pyrimidin-4-ylamino}-phenyl)-benzenesulfonamide formic acid salt

MS: calc: C₃₁H₃₅N₅O₆S (605.72) fnd: [MH⁺] 560.2

### 104. 4-Methoxy-N-(4-{6-[4-(2-pyrrolidin-1-yl-ethoxy)-phenyl]-pyrimidin-4-ylamino}-phenyl)-benzenesulfonamide formic acid salt

MS: calc: C₃₀H₃₃N₅O₆S (591.69) fnd: [MH⁺] 546.3

### 105. 4-Methyl-N-[4-(6-{4-[2-(4-methyl-piperazin-1-yl)-ethoxy]-phenyl}-pyrimidin-4-ylamino)-phenyl]-benzenesulfonamide formic acid salt

MS: calc: C₃₁H₃₅N₆O₅S (604.73) fnd: [MH⁺] 559.3

### 106. 4-Methyl-N-(4-{6-[4-(1-methyl-piperidin-4-ylmethoxy)-phenyl]-pyrimidin-4-ylamino}-phenyl)-benzenesulfonamide formic acid salt

MS: calc: C₃₁H₃₅N₅O₅S (589.72) fnd: [MH⁺] 544.2

### 107. 4-Fluoro-N-[4-(6-{4-[5-(1-methyl-piperidin-4-yl)-ethoxy]-phenyl}-pyrimidin-4-ylamino)-phenyl]-benzenesulfonamide formic acid salt

MS: calc: C₃₁H₃₄FN₅O₅S (607.71) fnd: [MH⁺] 5625

### 108. 4-Fluoro-N-(4-{6-[4-(1-methyl-piperidin-4-ylmethoxy)-phenyl]-pyrimidin-4-ylamino}-phenyl)-benzenesulfonamide formic acid salt

MS: calc: C₃₀H₃₂FN₅O₅S (593.68) fnd: [MH⁺] 548.2

### 109. 4-Fluoro-N-(4-{6-[4-(3-pyrrolidin-1-yl-propoxy)-phenyl]-pyrimidin-4-ylamino}-phenyl)-benzenesulfonamide formic acid salt

MS: calc: C₃₀H₃₂FN₅O₅S (593.68) fnd: [MH⁺] 548.3

### 110. 2-Fluoro-N-[4-(6-{4-[3-(4-methyl-piperazin-1-yl)-propoxy]-phenyl}-pyrimidin-4-ylamino)-phenyl]-benzenesulfonamide formic acid salt

MS: calc: C₃₁H₃₅FN₆O₅S (622.72) fnd: [MH⁺] 577.3

### 111. 2,6-Difluoro-N-[4-(6-{4-[3-(4-methyl-piperazin-1-yl)-propoxy]-phenyl}-pyrimidin-4-ylamino)-phenyl]-benzenesulfonamide formic acid salt

MS: calc: C₃₁H₃₄F₂N₅O₆S (640.71) fnd: [MH⁺] 595.3

### 112. 2,4-Difluoro-N-[4-(6-{4-[3-(4-methyl-piperazin-1-yl)-propoxy]-phenyl}-pyrimidin-4-ylamino)-phenyl]-benzenesulfonamide formic acid salt

MS: calc: C₃₁H₃₄F₂N₆O₅S (640.71) fnd: [MH⁺] 595.3

### 113. 4-Fluoro-N-[4-(6-{4-[3-(4-methyl-piperazin-1-yl)-propoxy]-phenyl}-pyrimidin-4-ylamino)-phenyl]benzenesulfonamide formic acid salt

MS: calc: C₃₁H₃₅FN₈O₅S (622.72) fnd: [MH⁺] 577.3

### 114. 2,4-Difluoro-N-(4-{6-[4-(1-methyl-piperidin-4-ylmethoxy)-phenyl]-pyrimidin-4-ylamino}phenyl)-benzenesulfonamide

MS: C₂₉H₂₉F₂N₅O₃S (565.65) fnd: [MH⁺] 566.2

### 115. 2,6-Difluoro-N-{4-[6-(2-methyl-2,3-dihydro-benzofuran-5-yl)-pyrimidin-4-ylamino]-phenyl}-benzenesulfonamide

MS: calc: C₂₅H₂₀F₂N₄O₃S (494.52) fnd: [MH⁺] 495.2

### 116. 2,6-Difluoro-N-{4-[6-(1-methyl-1H-pyrrol-3-yl)-pyrimidin-4-ylamino]-phenyl}-benzenesulfonamide

MS: calc: C₂₁H₁₇F₂N₅O₂S (441.46) fnd: [MH⁺] 442.0

### 117. 2,6-Difluoro-N-{4-[6-(4-methyl-thiophen-2-yl)-pyrimidin-4-ylamino]-phenyl}-benzenesulfonamide

MS: calc: C₂₁H₁₈F₂N₄O₂S₂ (458.51) fnd: [MH⁺] 459.0

### 118. 2,4-Difluoro-N-{4-[6-(1H-indol-5-yl)-pyrimidin-4-ylamino]-phenyl}-benzenesulfonamide

MS: calc: C₂₄H₁₇F₂N₅O₂S (477.50) fnd: [MH⁺] 478.2

### 119. 2,6-Difluoro-N-{4-[6-(1H-indol-5-yl)-pyrimidin-4-ylamino]-phenyl}-benzenesulfonamide

MS: calc: C₂₄H₁₇F₂N₅O₂S (477.50) fnd: [MH⁺] 478.2

### 120. 2-Fluoro-N-{4-[6-(1H-indol-5-yl)-pyrimidin-4-ylamino]-phenyl}-benzenesulfonamide

MS: calc: C₂₄H₁₈FN₅O₂S (459.51) fnd: [MH⁺] 460.2

### 121. 3-Fluoro-N-{4-[6-(1H-indol-5-yl)-pyrimidin-4-ylamino]-phenyl}-benzenesulfonamide

MS: calc: C₂₄H₁₈FN₅O₂S (459.51) fnd: [MH⁺] 460.2

### 122. 2,6-Difluoro-{4-[6-(1-methyl-1H-indol-3-yl)-pyrimidin-4-ylamino]-phenyl}-benzenesulfonamide

MS: calc: C₂₅H₁₉F₂N₅O₂S (491.52) fnd: [MH⁺] 492.3

### 123. 2,6-Difluoro-N-{4-[6-(1-methyl-1H-indol-5-yl)-pyrimidin-4-ylamino]-phenyl}-benzenesulfonamide

MS: calc: C₂₅H₁₉F₂N₅O₂S (491.52) fnd: [MH⁺] 492.3

### 124. 2-Fluoro-N-{4-[6-(1-methyl-1H-indol-5-yl)-pyrimidin-4-ylamino]-phenyl}-benzenesulfonamide

MS: calc: C₂₅H₂₀FN₅O₂S (473.53) fnd: [MH⁺] 474.3

### 125. 4-Methoxy-N-{4-[6-(1-methyl-1H-indol-3-yl)pyrimidin-4-ylamino]-phenyl}-benzenesulfonamide

MS: calc: C₂₅H₂₃N₅O₃S (485.57) fnd: [MH⁺] 486.3

### 126. 4-Methoxy-N-{4-[6-(1-methyl-1H-indol-5-yl)-pyrimidin-4-ylamino]-phenyl}-benzenesulfonamide

MS: calc: C₂₆H₂₆N₅O₃S (485.57) fnd: [MH⁺] 486.3

### 127. 2-Fluoro-N-{4-[6-(1-methyl-1H-indol-3-yl)-pyrimidin-4-ylamino]-phenyl}-benzenesulfonamide

MS: calc: C₂₅H₂₀FN₅O₂S (473.53) fnd: [MH⁺] 474.3

### 128. N-(4-{6-[1-(3-Dimethylamino-propyl)-1H-indol-5-yl]-pyrimidin-4-ylamino}-phenyl)-2,6-difluoro-benzenesulfonamide

MS: calc: C₂₉H₂₆F₂N₆O₂S (562.65) fnd: [MH⁺] 563.3

### 129. N-(4-{6-[1-(3-Dimethylamino-propyl)-1H-indol-5-yl]-pyrimidin-4-ylamino}-phenyl)-2-fluorobenzenesulfonamide

MS: calc: C₂₉H₂₉FN₆O₂S (544.66) fnd: [MH⁺] 545.3

### 130. N-(4-{6-[1-(2-Dimethylamino-ethyl)-1H-indol-5-yl]-pyrimidin-4-ylamino}-phenyl)-2-fluoro-benzenesulfonamide

MS: calc: C₂₈H₂₇FN₆O₂S (530.63) fnd: [MH⁺] 531.3

### 131. N-(4-[6-[1-(3-Dimethylamino-propyl)-1H-indol-5-yl]pyrimidin-4-ylamino]phenyl)-4-methoxy-benzenesulfonamide

MS: calc: C₃₀H₃₂N₆O₃S (556.69) fnd: [MH⁺] 557.4

### 132. N-(4-{6-[1-(2-Dimethylamino-ethyl)-1H-indol-5-yl]-pyrimidin-4-ylamino}-phenyl)-4-methoxy-benzenesulfonamide

MS: calc: C₂₉H₃₀N₅O₃S (542.66) fnd: [MH⁺] 543.4

### 133. N-(4-{6-[1-(3-Dimethylamino-propyl)-1H-indol-5-yl]-pyrimidin-4-ylamino}-phenyl)-2,4-difluorobenzenesulfonamide

MS: calc: C₂₉H₂₈F₂N₆O₂S (562.65) fnd: [M+NH₄⁺] 579.3

### 134. N-(4-{6-[1-(2-Dimethylamino-ethyl)-1H-indol-5-yl]-pyrimidin-4-ylamino}-phenyl)-2,4-difluoro-benzenesulfonamide

MS: calc: C₂₈H₂₆F₂N₆O₂S (548.62) fnd: [M+NH₄⁺] 565.4

### 135. N-(4-{6-[1-(3-Dimethylamino-propyl)-1H-indol-5-yl]-pyrimidin-4-ylamino}-phenyl)-4-methyl-benzenesulfonamide

MS: calc: C₃₀H₃₂N₆O₂S (540.69) fnd: [MH⁺] 541.4

### 136. N-(4-{6-[1-(2-Dimethylamino-ethyl)-1H-indol-5-yl]pyrimidin-4-ylamino}-phenyl)-4-methyl-benzenesulfonamide

MS: calc: C₂₉H₃₀N₆O₂S (526.67) fnd: [MH⁺] 527.4

### 137. 3-Chloro-N-(4-{6-[1-(3-dimethylamino-propyl)-1H-indol-5-yl]-pyrimidin-4-ylamino}-phenyl)-4-fluoro-benzenesulfonamide

MS: calc: C₂₉H₂₈ClFN₆O₂S (579.10) fnd: [MH⁺] 579.4

### 138. 3-Chloro-N-{4-[6-[1-(2-dimethylamino-ethyl)-1H-indol-5-yl]pyrimidin-4-ylamino]-phenyl}-4-fluoro-benzenesulfonamide

MS: calc: C₂₈H₂₆ClFN₆O₂S (565.07) fnd: [MH⁺] 565.3

### 139. N-(4-{6-[1-(2-Dimethylamino-ethyl)-1H-indol-5-yl]-pyrimidin-4-ylamino}-phenyl)-2,6-difluoro-benzenesulfonamide

MS: calc: C₂₈H₂₆F₂N₈O₂S (548.62) fnd: [MH⁺] 549.3

### 140. 2-Fluoro-N-(4-{6-[1-(2-pyrrolidin-1-yl-ethyl)-1H-indol-5-yl]pyrimidin-4-ylamino}-phenyl)-benzenesulfonamide formic acid salt

MS: calc: C₃₁H₃₁FN₆O₄S (602.69) fnd: [MH⁺] 557.3

### 141. 2-Fluoro-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₂H₃₄FN₇O₄S (631.73) fnd: [MH⁺] 586.3

### 142. 2,6-Difluoro-N-(4-{6-[1-(2-pyrrolidin-1-yl-ethyl)-1H-indol-5-yl]-pyrimidin-4-ylamino}-phenyl)-benzenesulfonamide formic acid salt

MS: calc: C₃₁H₃₀F₂N₆O₄S (620.68) fnd: [MH⁺] 5755

### 143. 2,6-Difluoro-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₂H₃₂F₂N₅O₄S (634.71) fnd: [MH⁺] 589.3

### 144. 2,6-Difluoro-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₂H₃₃F₂N₇O₄S (649.73) fnd: [MH⁺] 604.3

### 145. 4-Methoxy-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₃H₃₅N₈O₅S (628.76) fnd: [MH⁺] 583.3

### 146. 4-Methyl-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₃H₃₆N₅O₄S (612.76) fnd: [MH⁺] 567.3

### 147. 4-Methyl-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₃H₃₇N₇O₄S (627.77) fnd: [MH⁺] 582.4

### 148. 2,4-Difluoro-N-(4-{6-[1-(2-pyrrolidin-1-yl-ethyl)-1H-indol-5-yl]-pyrimidin-4-ylamino}-phenyl)-benzenesulfonamide formic acid salt

MS: calc: C₃₁H₃₀F₂N₆O₄S (620.68) fnd: [MH⁺] 575.3

### 149. 2,4-Difluoro-N-(4-{6-[1-(3-pyrrolidin-1-yl-propyl)-1H-indol-5-yl]pyrimidin-4-ylamino}-phenyl)-benzenesulfonamide formic acid salt

MS: calc: C₃₂H₃₂F₂N₅O₄S (634.71) fnd: [MH⁺] 589.3

### 150. 2,4-Difluoro-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₂H₃₃F₂N₇O₄S (649.73) fnd: [MH⁺] 604.3

### 151. 4-Fluoro-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₂H₃₃FN₆O₄S (616.72) fnd: [MH⁺] 571.3

### 152. 2-Fluoro-N-(4-{6-[1-(3-pyrrolidin-1-yl-propyl)-1H-indol-5-yl]-pyrimidin-4-ylamino}-phenyl)-benzenesulfonamide formic acid salt

MS: calc: C₃₂H₃₃FN₅O₄S (616.72) fnd: [MH⁺] 571.3

### 153. 2-Fluoro-N-[4-(6-{1-[3-(4-methyl-piperazin-1-yl)-propyl]-1H-indol-5-yl}-pyrimidin-4-ylamino)-phenyl]-benzenesulfonamide formic acid salt

MS: calc: C₃₃H₃₆FN₇O₄S (645.76) fnd: [MH⁺] 600.3

### 154. 2,6-Difluoro-N-[4-(6-{1-[3-(4-methyl-piperazin-1-yl)-propyl]-1H-indol-5-yl}-pyrimidin-4-ylamino)-phenyl]-benzenesulfonamide formic acid salt

MS: calc: C₃₃H₃₅F₂N₇O₄S (663.75) fnd: [MH⁺] 618.3

### 155. 4-Methoxy-N-(4-{6-[1-(2-pyrrolidin-1-yl-ethyl)-1H-indole-5-yl]-pyrimidin-4-ylamino}-phenyl)-benzenesulfonamide formic acid salt

MS: calc: C₃₂H₃₄N₆O₅S (614.73) fnd: [MH⁺] 569.3

### 156. 4-Methoxy-N-[4-(6-{1-[3-(4-methyl-piperazin-1-yl)-propyl]-1H-indol-5-yl}-pyrimidin-4-ylamino)-phenyl]-benzenesulfonamide formic acid salt

MS: calc: C₃₄H₃₉N₇O₅S (657.80) fnd: [MH⁺] 612.3

### 157. 4-Methoxy-N-[4-(6-{1-[3-(4-methyl-piperazin-1-yl)-propyl]-1H-indol-5-yl}-pyrimidin-4-ylamino)-phenyl]benzenesulfonamide formic acid salt

MS: calc: C₃₃H₃₇N₇O₅S (643.77) fnd: [MH⁺] 598.3

### 158. 4-Methyl-N-(4-{6-[1-(2-pyrrolidin-1-yl-ethyl)-1H-indol-5-yl]-pyrimidin-4-ylamino}-phenyl)-benzenesulfonamide formic acid salt

MS: calc: C₃₂H₃₄N₆O₄S (598.73) fnd: [MH⁺] 553.3

### 159. 4-Methyl-N-[4-(6-{1-[3-(4-methyl-piperazin-1-yl)-propyl]-1H-indol-5-yl}-pyrimidin-4-ylamino)-phenyl]-benzenesulfonamide formic acid salt

MS: calc: C₃₄H₃₉N₇O₄S (641.80) fnd: [MH⁺] 596.3

### 160. 2,4-Difluoro-N-[4-(6-{1-[3-(4-methyl-piperazin-1-yl)-propyl]1H-indol-5-yl}-pyrimidin-4-ylamino)-phenyl]-benzenesulfonamide formic acid salt

MS: calc: C₃₃H₃₅F₂N₇O₄S (663.75) fnd: [MH⁺] 618.3

### 161. 3-Chloro-4-fluoro-N-(4-{6-[1-(2-pyrrolidin-1-yl-ethyl)-1H-indol-5-yl]pyrimidin-4-ylamino}-phenyl)-benzenesulfonamide formic acid salt

MS: calc: C₃₁H₃₀ClFN₆O₄S (637.14) fnd: [MH⁺] 591.2

### 162. 3-Chloro-4-fluoro-N-[4-(6-{1-[3-(4-methyl-piperazin-1-yl)-propyl]-1H-indol-5-yl}-pyrimidin-4-ylamino)-phenyl]-benzenesulfonamide formic acid salt

MS: calc: C₃₃H₃₅ClFN₇O₄S (680.21) fnd: [MH⁺] 634.3

### 163. 2-Fluoro-N-[4-(6-{1-[3-(4-methyl-piperazin-1-yl)-propyl]-1H-indol-3-yl}-pyrimidin-4-ylamino)-phenyl]-benzenesulfonamide formic acid salt

MS: calc: C₃₃H₃₅FN₇O₄S (645.76) fnd: [MH⁺] 600.3

### 164. 2,4-Difluoro-N-[4-(6-{1-[3-(4-methyl-piperazin-1-yl)-propyl]-1H-indol-3-yl}-pyrimidin-4-ylamino)-phenyl]-benzenesulfonamide formic acid salt

MS: calc: C₃₃H₃₅F₂N₇O₄S (663.75) fnd: [MH⁺] 618.4

### 165. 4-Methoxy-N-[4-(6-{1-[3-(4-methyl-piperazin-1-yl)-propyl]-1H-indol-3-yl}-pyrimidin-4-ylamino)-phenyl]-benzenesulfonamide formic acid salt

MS: calc: C₃₄H₃₉N₇O₅S (657.80) fnd: [MH⁺] 612.3

### 166. N-(4-{6-[1-(3-Diethylamino-propyl)-1H-indol-5-yl]-pyrimidin-4-ylamino}-phenyl)-2,6-difluoro-benzenesulfonamide formic acid salt

MS: calc: C₃₂H₃₄F₂N₆O₄S (636.73) fnd: [MH⁺] 591.3

### 167. N-(4-{6-[1-(3-Diethylamino-propyl)-1H-indol-5-yl]pyrimidin-4-ylamino}-phenyl)-2-fluoro-benzenesulfonamide formic acid salt

MS: calc: C₃₂H₃₅FN₆O₄S (618.74) fnd: [MH⁺] 573.3

### 168. N-(4-{6-[1-(3-Diethylamino-propyl)-1H-indol-5-yl]-pyrimidin-4-ylamino}-phenyl)-2,4-difluoro-benzenesulfonamide formic acid salt

MS: calc: C₃₂H₃₄F₂N₆O₄S (636.73) fnd: [MH⁺] 591.3

### 169. N-(4-{6-[1-(3-Diethylamino-propyl)-1H-indol-5-yl]-pyrimidin-4-ylamino}-phenyl)-4-fluoro-benzenesulfonamide formic acid salt

MS: calc: C₃₂H₃₅FN₆O₄S (618.74) fnd: [MH⁺] 573.3

### 170. N-(4-{6-[1-(3-Diethylamino-propyl)-1H-indol-5-yl]-pyrimidin-4-ylamino}-phenyl)-4-methoxy-benzenesulfonamide formic acid salt

MS: calc: C₃₃H₃₈N₆O₅S (630.77) fnd: [MH⁺] 585.3

### 171. N-(4-{6-[1-(3-Diethylamino-propyl)-1H-indol-5-yl]pyrimidin-4-ylamino}-phenyl)-4-methyl-benzenesulfonamide formic acid salt

MS: calc: C₃₃H₃₈N₈O₄S (614.77) fnd: [MH⁺] 569.3

### 172. N-(4-{6-[1-(3-Dimethylamino-propyl)-1H-indol-5-yl]-pyrimidin-4-ylamino}-phenyl)-2,4-difluoro-benzenesulfonamide

MS: calc: C₂₉H₂₉F₂N₆O₂S (562.65) fnd: [MH⁺] 563.3

### 173. N-(4-{6-[1-(3-Diethylamino-propyl)-1H-indol-3-yl]-pyrimidin-4-ylamino}-phenyl)-2,6-difluoro-benzenesulfonamide formic acid salt

MS: calc: C₃₂H₃₄F₂N₈O₄S (636.73) fnd: [MH⁺] 591.4

### 174. N-(4-{6-[1-(3-Diethylamino-propyl)-1H-indol-3-yl]-pyrimidin-4-ylamino}-phenyl)-2-fluoro-benzenesulfonamide formic acid salt

MS: calc: C₃₂H₃₅FN₈O₄S (618.74) fnd: [MH⁺] 573.4

### 175. N-(4-{6-[1-(3-Diethylamino-propyl)-1H-indol-3-yl]-pyrimidin-4-ylamino}-phenyl)-2,4-difluoro-benzenesulfonamide formic acid salt

MS: calc: C₃₂H₃₄F₂N₈O₄S (636.73) fnd: [MH⁺] 591.3

### 176. N-(4-{6-[1-(3-Diethylamino-propyl)-1H-indol-3-yl]-pyrimidin-4-ylamino}-phenyl)-4-methoxy-benzenesulfonamide formic acid salt

MS: calc: C₃₃H₃₈N₆O₅S (630.77) fnd: [MH⁺] 585.4

### 177. N-(4-{6-[1-(2-Diethylamino-ethyl)-1H-indol-5-yl]-pyrimidin-4-ylamino}-phenyl)-2-fluorobenzenesulfonamide formic acid salt

MS: calc: C₃₁H₃₃FN₆O₄S (604.71) fnd: [MH⁺] 559.3

### 178. N-(4-{6-[1-(2-Diethylamino-ethyl)-1H-indol-5-yl]-pyrimidin-4-ylamino}-phenyl)-4-methyl-benzenesulfonamide formic acid salt

MS: calc: C₃₂N₃₅N₆O₄S (600.75) fnd: [MH⁺] 555.3

### 179. 3-Chloro-N-(4-{6-[1-(2-diethylamino-ethyl)-1H-indol-5-yl]-pyrimidin-4-ylamino}-phenyl)-4-fluoro-benzenesulfonamide formic acid salt

MS: calc: C₃₁H₃₂ClFN₆O₄S (639.15) fnd: [MH⁺] 5935

### 180. N-(4-{6-[1-(2-Diethylamino-ethyl)-1H-indol-5-yl]-pyrimidin-4-ylamino}-phenyl)-2,4-difluoro-benzenesulfonamide formic acid salt

MS: calc: C₃₁H₃₂F₂N₆O₄S (622.7) fnd: [MH⁺] 577.3

### 181. N-(4-{6-[1-(2-Diethylamino-ethyl)-1H-indol-5-yl]-pyrimidin-4-ylamino}-phenyl)-2,6-difluoro-benzenesulfonamide formic acid salt

MS: calc: C₃₁H₃₂F₂N₅O₄S (622.7) fnd: [MH⁺] 577.3

### 182. N-(4-{6-[1-(2-Diethylamino-ethyl)-1H-indol-5-yl]-pyrimidin-4-ylamino}-phenyl)-4-methoxy-benzenesulfonamide formic acid salt

MS: calc: C₃₂H₃₆N₆O₅S (616.74) fnd: [MH⁺] 571.3

### 183. N-(4-{6-[1-(2-Diethylamino-ethyl)-1H-indol-5-yl]-pyrimidin-4-ylamino}-phenyl)-4-fluoro-benzenesulfonamide formic acid salt

MS: calc: C₃₁H₃₃FN₆O₄S (604.71) fnd: [MH⁺] 559.3

### 184. N-(4-{6-[1-(2-Diethylamino-ethyl)-1H-indol-5-yl]-pyrimidin-4-ylamino}-phenyl)-3-fluoro-4-methoxybenzenesulfonamide formic acid salt

MS: calc: C₃₂H₃₅FN₆O₅S (634.74) fnd: [MH⁺] 589.3

### 185. N-(4-{6-[1-(2-Diethylamino-ethyl)-1H-indol-5-yl]-pyrimidin-4-ylamino}-phenyl)-2-fluoro-4-methyl-benzenesulfonamide formic acid salt

MS: calc: C₃₂H₃₅FN₆O₄S (618.74) fnd: [MH⁺] 573.3

### 186. N-(4-{6-[1-(4-Diethylamino-butyl)-1H-indol-5-yl]-pyrimidin-4-ylamino}-phenyl)-2-fluorobenzenesulfonamide formic acid salt

MS: C₃₃H₃₇FN₆O₄S (632.76) fnd: [MH⁺] 587.4

### 187. N-(4-{6-[1-(4-Diethylamino-butyl-1H-indol-5-yl]-pyrimidin-4-ylamino}-phenyl)-4-methyl-benzenesulfonamide formic acid salt

MS: calc: C₃₄H₄₀N₆O₄S (628.8) fnd: [MH⁺] 583.4

### 188. 3-Chloro-N-(4-{6-[1-(4-diethylamino-butyl)-1H-indol-5-yl]-pyrimidin-4-ylamino}-phenyl)-4-fluoro-benzenesulfonamide formic acid salt

MS: calc: C₃₃H₃₅ClFN₆O₄S (667.21) fnd: [MH⁺] 621.3

### 189. N-(4-{6-[1-(4-Diethylamino-butyl)-1H-indol-5-yl]-pyrimidin-4-ylamino}-phenyl)-2,4-difluoro-benzenesulfonamide formic acid salt

MS: calc: C₃₃H₃₆F₂N₆O₄S (650.75) fnd: [MH⁺] 605.3

### 190. N-(4-{6-[1-(4-Diethylamino-butyl)-1H-indol-5-yl]pyrimidin-4-ylamino}-phenyl)-2,6-difluoro-benzenesulfonamide formic acid salt

MS: calc: C₃₃H₃₆F₂N₆O₄S (650.75) fnd: [MH⁺] 605.3

### 191. N-(4-{6-[1-(4-Diethylamino-butyl)-1H-indol-5-yl]-pyrimidin-4-ylamino}-phenyl)-4-methoxy-benzenesulfonamide formic acid salt

MS: calc: C₃₄H₄₀N₆O₅S (644.8) fnd: [MH⁺] 599.4

### 192. N-(4-{6-[1-(4-Diethylamino-butyl)-1H-indol-5-yl]-pyrimidin-4-ylamino}-phenyl)-4-fluorobenzenesulfonamide formic acid salt

MS: calc: C₃₃H₃₇FN₆O₄S (632.76) fnd: [MH⁺] 587.4

### 193. N-(4-{6-[1-(4-Diethylamino-butyl)-1H-indol-5-yl]pyrimidin-4-ylamino}-phenyl)-3-fluoro-4-methoxy-benzenesulfonamide formic acid salt

MS: calc: C₃₄H₃₉FN₆O₅S (662.79) fnd: [MH⁺] 617.3

### 194. N-(4-{6-[1-(4-Diethylamino-butyl)-1H-indol-5-yl]-pyrimidin-4-ylamino}-phenyl)-2-fluoro-4-methyl-benzenesulfonamide formic acid salt

MS: calc: C₃₄H₃₉FN₆O₄S (646.79) fnd: [MH⁺] 601.3

### 195. 2-Fluoro-N-(4-{6-[1-(1-methyl-piperidin-4-ylmethyl)-1H-indol-5-yl]-pyrimidin-4-ylamino}-phenyl)-benzenesulfonamide formic acid salt

MS: calc: C₃₂H₃₃FN₆O₄S (616.72) fnd: [MH⁺] 571.3

### 196. 4-Methyl-N-4-{6-[1-(1-methyl-piperidin-4-ylmethyl)-indol-5-yl]-pyrimidin-4-ylamino}-phenyl)-benzenesulfonamide formic acid salt

MS: calc: C₃₃H₃₅N₆O₄S (612.76) fnd: [MH⁺] 567.4

### 197. 3-Chloro-4-fluoro-N-(4-{6-[1-(1-methyl-piperidin-4-ylmethyl)-1H-indol-5-yl]pyrimidin-4-ylamino}-phenyl)-benzenesulfonamide formic acid salt

MS: calc: C₃₂H₃₂ClFN₆O₄S (651.17) fnd: [MH⁺] 605.3

### 198. 2,4-Difluoro-N-(4-{6-[1-(1-methyl-piperidin-4-ylmethyl)-1H-indol-5-yl]-pyrimidin-4-ylamino}-phenyl)-benzenesulfonamide formic acid salt

MS: calc: C₃₂H₃₂F₂N₆O₄S (634.71) fnd: [MH⁺] 589.3

### 199. 2,6-Difluoro-N-(4-{6-[1-(1-methyl-piperidin-4-ylmethyl)-1H-5-yl]-pyrimidin-4-ylamino}-phenyl)-benzenesulfonamide formic acid salt

MS: calc: C₃₂H₃₂F₂N₈O₄S (634.71) fnd: [MH⁺] 507.6

### 200. 4-Methoxy-N-4-{6-[1-(1-methyl-piperidin-4-ylmethyl)-1H-indol-5-yl]-pyrimidin-4-ylamino}-phenyl)-benzenesulfonamide formic acid salt

MS: calc: C₃₃H₃₅N₆O₅S (628.76) fnd: [MH⁺] 583.4

### 201. 4-Fluoro-N-(4-{6-[1-(1-methyl-piperidin-4-ylmethyl)-1H-indol-5-yl]-pyrimidin-4-ylamino}-phenyl)-benzenesulfonamide formic acid salt

MS: calc: C₃₂H₃₃FN₆O₄S (616.72) fnd: [MH⁺] 571.3

### 202. 3-Fluoro-4-methoxy-N-(4-{6-[1-(1-methyl-piperidin-4-ylmethyl)-1H-indol-5-yl]-pyrimidin-4-ylamino}-phenyl)-benzenesulfonamide formic acid salt

MS: calc: C₃H₃₅FN₆O₅S (646.75) fnd: [MH⁺] 601.3

### 203. 2-Fluoro-4-methyl-N-(4-{6-[1-(1-methyl-piperidin-4-ylmethyl)-1H-indol-5-yl]-pyrimidin-4-ylamino}-phenyl)-benzenesulfonamide formic acid salt

MS: calc: C₃₃H₃₅FN₆O₄S (630.75) fnd: [MH⁺] 585.3

### 204. 2-Fluoro-N-[4-(6-{1-[2-(1-methyl-piperidin-4-yl)-ethyl]-1H-indol-5-yl}-pyrimidin-4-ylamino)-phenyl]-benzenesulfonamide formic acid salt

MS: calc: C₃₃H₃₅FN₆O₄S (630.75) fnd: [MH⁺] 585.3

### 205. 4-Methyl-N-[4-(6-{1-[2-(1-methyl-piperidin-4-yl)-ethyl]-1H-indol-5-yl}-pyrimidin-4-ylamino)-phenyl]-benzenesulfonamide

MS: calc: C₃₃H₃₈N₆O₂S (580.76) fnd: [MH⁺] 581.3

### 206. 3-Chloro-4-fluoro-N-[4-(6-{1-[2-(1-methyl-piperidin-4-yl)-ethyl]-1H-indol-5-yl}-pyrimidin-4-ylamino)-phenyl]-benzenesulfonamide formic acid salt

MS: calc: C₃₃H₃₄ClFN₆O₄S (665.19) fnd: [MH⁺] 619.3

### 207. 2,4-Difluoro-N-[4-(6-{1-[2-(1-methyl-piperidin-4-yl)-ethyl]-1H-indol-5-yl}-pyrimidin-4-ylamino)-phenyl]-benzenesulfonamide formic acid salt

MS: calc: C₃₃H₃₄F₂N₅O₄S (648.74) fnd: [MH⁺] 603.3

### 208. 2,6-Difluoro-N-[4-(6-{1-[2-(1-methyl-piperidin-4-yl)-ethyl]-1H-indol-5-yl}-pyrimidin-4-ylamino)-phenyl]-benzenesulfonamide formic acid salt

MS: calc: C₃₃H₃₄F₂N₆O₄S (648.74) fnd: [MH⁺] 603.3

### 209. 4-Methoxy-N-[4-(6-{1-[2-(1-methyl-piperidin-4-yl)-ethyl]-1H-indol-5-yl}-pyrimidin-4-ylamino)-phenyl]-benzenesulfonamide formic acid salt

MS: calc: C₃₄H₃₅N₈O₅S (642.78) fnd: [MH⁺] 597.3

### 210. 4-Fluoro-N-[4-(6-{1-[2-(1-methyl-piperidin-4-yl)-ethyl]-1H-indol-5-yl}-pyrimidin-4-ylamino)-phenyl]-benzenesulfonamide

MS: calc: C₃₂H₃₃FN₆O₂S (584.72) fnd: [MH⁺] 585.3

### 211. 3-Fluoro-4-methoxy-N-[4-(6-{1-[2-(1-methyl-piperidin-4-yl)-ethyl]-1H-indol-5-yl}-pyrimidin-4-ylamino)-phenyl]-benzenesulfonamide

MS: calc: C₃₃H₃₅FN₆O₃S (614.75) fnd: [MH⁺] 615.3

### 212. 2-Fluoro-4-methyl-N-[4-(6-{1-[2-(1-methyl-piperidin-4-yl)-ethyl]-1H-indol-5-yl}-pyrimidin-4-ylamino)-phenyl]-benzenesulfonamide

MS: calc: C₃₃H₃₅FN₅O₂S (598.75) fnd: [MH⁺] 599.3

### 213. 2,4-Difluoro-N-(4-{6-[3-fluoro-4-(1-methyl-piperidin-4-ylmethoxy)-phenyl]-pyrimidin-4-ylamino}-phenyl)-benzenesulfonamide formic acid salt

MS: calc: C₃₀H₃₀F₃O₅S (629.66) fnd: [MH⁺] 584.1

### 214. 3-Fluoro-N-(4-{6-[3-fluoro-4-(1-methyl-piperidin-4-ylmethoxy)-phenyl]-pyrimidin-4-ylamino}-phenyl)-4-methoxy-benzenesulfonamide formic acid salt

MS: calc: C₃₁H₃₃F₂N₅O₆S (641.70) fnd: [MH⁺] 596.2

### 215. 2-Fluoro-N-(4-{6-[3-fluoro-4-(1-methyl-piperidin-4-ylmethoxy)-phenyl]-pyrimidin-4-ylamino}-phenyl)-4-methyl-benzenesulfonamide formic acid sail

MS: calc: C₃₁H₃₃F₂N₅O₅S (625.70) fnd: [MH⁺] 580.2

### 216. 2-Fluoro-N-[4-(6-{3-fluoro-4-[2-(1-methyl-piperidin-4-yl)-ethoxy]-phenyl}-pyrimidin-4-ylamino)-phenyl]-benzenesulfonamide formic acid salt

MS: calc: C₃₁H₃₃F₂N₅O₅S (625.70) fnd: [MH⁺] 580.2

### 217. 2,4-Difluoro-N-[4-(6-{3-fluoro-4-[2-(1-methyl-piperidin-4-yl)-ethoxy]-phenyl}-pyrimidin-4-ylamino)-phenyl]-benzenesulfonamide formic acid salt

MS: calc: C₃₁H₃₂F₃N₅O₅S (643.69) fnd: [MH⁺] 598.2

### 218. 2,6-Difluoro-N-[4-(6-{3-fluoro-4-[2-(1-methyl-piperidin-4-yl)-ethoxy]-phenyl}-pyrimidin-4-ylamino)-phenyl]-benzenesulfonamide formic acid salt

MS: calc: C₃₁H₃₂F₃N₅O₅S (643.69) fnd: [MH⁺] 598.2

### 219. N-[4-(6-{3-Fluoro-4-[2-(1-methyl-piperidin-4-yl)-ethoxy]-phenyl}-pyrimidin-4-ylamino)-phenyl]-methoxy-benzenesulfonamide formic acid salt

MS: calc: C₃₂H₃₈FN₅O₆S (637.74) fnd: [MH⁺] 592.2

### 220. 3-Fluoro-N-[4-(6-{3-fluoro-4-[2-(1-methyl-piperidin-4-yl)-ethoxy]-phenyl}-pyrimidin-4-ylamino)-phenyl]-4-methoxy-benzenesulfonamide formic acid salt

MS: calc: C₃₂H₃₅F₂N₅O₆S (655.73) fnd: [MH⁺] 610.2

### 221. 4-Fluoro-N-[4-(6-{3-fluoro-4-[2-(1-methyl-piperidin-4-yl)-ethoxy]-phenyl}-pyrimidin-4-ylamino)-phenyl]benzenesulfonamide formic acid salt

MS: calc: C₃₁H₃₃F₂N₅O₅S (625.70) fnd: [MH⁺] 580.3

### 222. 2-Fluoro-N-[4-(6-{3-fluoro-4-[2-(1-methyl-piperidin-4-yl)-ethoxy]-phenyl}-pyrimidin-4-ylamino)-phenyl]4-methyl-benzenesulfonamide formic acid salt

MS: calc: C₃₂H₃₅F₂N₅O₅S (639.73) fnd: [MH^{+]} 594.2

### 223. 2-Fluoro-4-methyl-N-(4-{6-[4-(1-methyl-piperidin-4-ylmethoxy)-phenyl]-pyrimidin-4-ylamino}-phenyl)-benzenesulfonamide formic acid salt

MS: calc: C₃₁H₃₄FN₆O₅S (607.71) fnd: [MH⁺] 562.2

### 224. 3-Fluoro-4-methoxy-N-(4-{6-[4-(1-methyl-piperidin-4-ylmethoxy)-phenyl]-pyrimidin-4-ylamino}-phenyl)-benzenesulfonamide formic acid salt

MS: calc: C₃₁H₃₄FN₅O₆S (623.71) fnd: [MH⁺] 579.2

### 225. 2,6-Difluoro-N-(4-{6-[3-fluoro-4-(1-methyl-piperidin-4-ylmethoxy)-phenyl]-pyrimidin-4-ylamino}-phenyl)-benzenesulfonamide formic acid salt

MS: calc: C₃₀H₃₀F₃N₅O₅S (629.66) fnd: [MH⁺] 584.2

### 226. 6-Fluoro-N-(4-{6-[3-fluoro-4-(1-methyl-piperidin-4-ylmethoxy)-phenyl]-pyrimidin-4-ylamino}-phenyl)-benzenesulfonamide formic acid salt

MS: calc: C₃₀H₃₁F₂N₅O₅S (611.67) fnd: [MH⁺] 566.2

### 227. N-(4-{6-[3-Fluoro-4-(1-methyl-piperidin-4-ylmethoxy)-phenyl]-pyrimidin-4-ylamino}-phenyl)-4-methoxy-benzenesulfonamide formic acid salt

MS: calc: C₃₁H₃₄FN₅O₆S (623.71) fnd: [MH⁺] 578.1

### 228. 2,6-Difluoro-N-(4-{6-[3-methyl-4-(1-methyl-piperidin-4-ylmethoxy)-phenyl]-pyrimidin-4-ylamino}-phenyl)-benzenesulfonamide formic acid salt

MS: calc: C₃₁H₃₃F₂N₅O₅S (625.70) fnd: [MH⁺] 580.2

### 229. 6-Fluoro-(4-{6-[3-methyl-4-(1-methyl-piperidin-4-ylmethoxy)-phenyl]-pyrimidin-4-ylamino}-phenyl)-benzenesulfonamide formic acid salt

MS: calc: C₃₁H₃₄FN₅O₅S (607.71) fnd: [MH⁺] 562.2

### 230. 2,4-Difluoro-N-(4-{6-[3-methyl-4-(1-methyl-piperidin-4-ylmethoxy)-phenyl]-pyrimidin-4-ylamino}-phenyl)-benzenesulfonamide formic acid salt

MS: calc: C₃₁H₃₃F₂N₅O₅S (625.70) fnd: [MH⁺] 580.2

### 231. N-(4-{6-[3-methyl-4-(1-methyl-piperidin-4-ylmethoxy)-phenyl]-pyrimidin-4-ylamino}-phenyl)-4-methoxy-benzenesulfonamide formic acid salt

MS: calc: C₃₂H₃₇N₅O₆₋S (619.75) fnd: [MH⁺] 574.3

### 232. 3-Fluoro-4-methoxy-N-(4-{6-[3-methyl-4-(1-methyl-piperidin-4-ylmethoxy)-phenyl]-pyrimidin-4-ylamino}-phenyl)-benzenesulfonamide formic acid salt

MS: calc: C₃₂H₃₅FN₅O₆S (637.74) fnd: [MH⁺] 592.2

### 233. 2,6-Difluoro-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₁H₃₃F₂N₅O₆S (641.70) fnd: [MH⁺] 596.1

### 234. 6-Fluoro-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₁H₃₄FN₅O₆S (623.71) fnd: [MH⁺] 578.2

### 235. 2,4-Difluoro-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₁H₃₃F₂N₅O₆S (641.70) fnd: [MH⁺] 596.2

### 236. 4-Methoxy-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₂H₃₇N₅O₇S (635.74) fnd: [MH⁺] 590.2

### 237. 4-Methyl-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₂H₃₇N₅O₅S (619.75) fnd: [MH⁺] 574.2

### 238. 2-Fluoro-N-(4-{6-[3-methoxy-4-(1-methyl-piperidin-4-ylmethoxy)-phenyl]-pyrimidin-4-ylamino}-phenyl)-4-methyl-benzenesulfonamide formic acid salt

MS: calc: C₃₂H₃₆FN₅O₅S (637.74) fnd: [MH⁺] 592.2

### 239. 3-Fluoro-4-methoxy-N-(4-{6-[3-methoxy-4-(1-methyl-piperidin-4-ylmethoxy)-phenyl]-pyrimidin-4-ylamino}-phenyl)-benzenesulfonamide formic acid salt

MS: calc: C₃₂H₃₆FN₅O₇S (653.74) fnd: [MH⁺] 608.2

### 240. 2-Fluoro-N-{4-[6-(1H-indol-5-yl)-pyrimidin-4-ylamino]-phenyl}-4-methyl-benzenesulfonamide formic acid salt

MS: calc: C₂₅H₂₂FN₅O₄S (519.56) fnd: [MH⁺] 474.2

### 241. 2-Fluoro-4-methyl-N-{4-[6-(1-methyl-1H-indol-5-yl)-pyrimidin-4-ylamino]-phenyl}-benzenesulfonamide formic acid salt

MS: calc: C₂₇H₂₄FN₅O₄S (533.59) fnd: [MH⁺] 488.2

### 242. N-(4-{6-[1-(2-Dimethylamino-ethyl)-1H-indol-5-yl]-pyrimidin-4-ylamino}-phenyl)-3-fluoro-4-methoxy-benzenesulfonamide formic acid salt

MS: calc: C₃₀H₃₁FN₅O₅S (606.68) fnd: [MH⁺] 561.2

### 243. N-(4-{6-[1-(2-Dimethylamino-ethyl)-1H-indol-5-yl]-pyrimidin-4-ylamino}-phenyl)-2-fluoro-4-methyl-benzenesulfonamide formic acid salt

MS: calc: C₃₀H₃₁FN₆O₄S (590.68) fnd: [MH⁺] 545.2

### 244. N-(4-{6-[1-(2-Dimethylamino-propyl)-1H-indol-5-yl]-pyrimidin-4-ylamino}-phenyl)-3-fluoro-4-methoxy-benzenesulfonamide formic acid salt

MS: calc: C₃₁H₃₃FN₆O₅S (620.71) fnd: [MH⁺] 575.2

### 245. N-(4-{6-[1-(2-Dimethylamino-propyl)-1H-indol-5-yl]-pyrimidin-4-ylamino}-phenyl)-2-fluoro-4-methyl-benzenesulfonamide formic acid salt

MS: calc: C₃₁H₃₃FN₆O₄S (604.71) fnd: [MH⁺] 559.2

### 246. N-(4-{6-[1-(2-Diethylamino-propyl)-1H-indol-5-yl]-pyrimidin-4-ylamino}-phenyl)-3-fluoro-4-methoxy-benzenesulfonamide formic acid salt

MS: calc: C₃₃H₃₇FN₈O₅S (648.76) fnd: [MH⁺] 603.2

### 247. N-(4-{6-[1-(2-Diethylamino-propyl)-1H-indol-5-yl]-pyrimidin-4-ylamino}-phenyl)-2-fluoro-4-methyl-benzenesulfonamide formic acid salt

MS: calc: C₃₃H₃₇FN₆O₄S (632.76) fnd: [MH⁺] 5875

### 248. 2-Fluoro-4-methyl-N-(4-{6-[1-(2-pyrrolidin-1-yl-ethyl)-1H-indol-5-yl]-pyrimidin-4-ylamino}-phenyl)-benzenesulfonamide formic acid salt

MS: calc: C₃₂H₂₂FN₆O₄S (616.72) fnd: [MH⁺] 5712.

### 249. 2-Fluoro-4-methyl-N-(4-{6-[1-(2-pyrrolidin-1-yl-propyl)-1H-indol-5-yl]-pyrimidin-4-ylamino}-phenyl)-benzenesulfonamide formic acid salt

MS: calc: C₃₃H₃₅FN₈O₄S (630.75) fnd: [MH⁺] 585.3

### 250. 2-Fluoro-4-methyl-N-[4-(6-{1-[2-(4-methyl-piperazin-1-yl)-ethyl]-1H-indol-5-yl}-pyrimidin-4-ylamino)-phenyl]-benzenesulfonamide formic acid salt

MS: calc: C₃₃H₃₆FN₇O₄S (645.76) fnd: [MH⁺] 600.3

### 251. 2-Fluoro-4-methyl-N-[4-(6-{1-[2-(4-methyl-piperazin-1-yl)-propyl]-1H-indol-5-yl}-pyrimidin-4-ylamino)-phenyl]-benzenesulfonamide formic acid salt

MS: calc: C₃₄H₃₈FN₇O₄S (659.79) fnd: [MH⁺] 614.3

### 252. N-(4-{6-[1-(3-Dimethylamino-propyl)-1H-benzoimidazol-5-yl]-pyrimidin-4-ylamino}-phenyl)-2-fluoro-benzenesulfonamide formic acid sail

MS: calc: C₂₉H₃₀FN₇O₄S (591.67) fnd: [MH⁺] 546.3

### 253. N-(4-{6-[1-(3-Dimethylamino-propyl)-1H-benzoimidazol-5-yl]-pyrimidin-4-ylamino}-phenyl)-2,6-difluoro-benzenesulfonamide formic acid salt

MS: calc: C₂₉H₂₉F₂N₇O₄S (609.66) fnd: [MH⁺] 564.3

### 254. N-(4-{6-[1-(3-Dimethylamino-propyl)-1H-benzoimidazol-5-yl]-pyrimidin-4-ylamino}-phenyl)-2-fluoro-4-methyl-benzenesulfonamide formic acid salt

MS: calc: C₃₀H₃₂FN₇O₄S (605.70) fnd: [MH⁺] 560.3

### 255. N-(4-{6-[1-(3-Dimethylamino-propyl)-1H-benzoimidazol-5-yl]-pyrimidin-4-ylamino}-phenyl)-4-methoxy-benzenesulfonamide formic acid salt

MS: calc: C₃₀H₃₃N₇O₅S (603.71) fnd: [MH⁺] 558.3

### 256. N-(4-{6-[1-(2-Dimethylamino-ethyl)-1H-indazol-5-yl]-pyrimidin-4-ylamino}-phenyl)-2-fluoro-benzenesulfonamide formic acid salt

MS: calc: C₂₈H₂₆FN₇O₄S (577.64) fnd: [MH⁺] 532.3

### 257. N-(4-{6-[1-(2-Dimethylamino-ethyl)-1H-indazol-5-yl]-pyrimidin-4-ylamino}-phenyl)-2,6-difluoro-benzenesulfonamide formic acid salt

MS: calc: C₂₆H₂₇F₂N₇O₄S (595.63) fnd: [MH⁺] 550.3

### 258. N-(4-{6-[1-(2-Dimethylamino-ethyl)-1H-indazol-5-yl]pyrimidin-4-ylamino}-phenyl)-2-fluoro-4-methyl-benzenesulfonamide formic acid salt

MS: calc: C₂₉H₃₀FN₇O₄S (591.67) fnd: [MH⁺] 546.3

### 259. N-(4-{6-[1-(2-Dimethylamino-ethyl)-1H-indazol-5-yl]-pyrimidin-4-ylamino}-phenyl)-4-methyl-benzenesulfonamide formic acid salt

MS: calc: C₂₉H₃₁N₇O₄S (573.68) fnd: [MH⁺] 528.3

### 260. N-(4-{6-[1-(2-Dimethylamino-ethyl)-1H-indazol-yl]-pyrimidin-4-ylamino}-phenyl)-4-methoxy-benzenesulfonamide formic acid salt

MS: calc: C₂₉H₃₁N₇O₅S (589.68) fnd: [MH⁺] 544.3

### Starting Materials and Intermediates:

Example of the synthesis of intermediates according to step 1, step 2, step 3, step 4 and step 7 (compare reaction scheme 1):

### A1. 3-(2-Methyl-2,3-dihydro-benzofuran-5-yl)-3-oxo-propionic acid ethylester

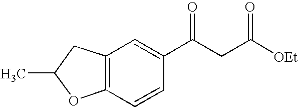

A solution of sodium hydride (60%, 12.9 g, 323 mmol) and diethyl carbonate (33 ml, 269 mmol) in absolute toluene (500 ml) is stirred at room temperature for 0.5 h. After stirring under reflux for 10 min a solution of 1-(2-Methyl-2,3-dihydro-benzofuran-5-yl)-ethanone (50 g, 269 mmol) in absolute toluene (250 ml) is slowly added and the reaction mixture is stirred again for 1 h under reflux. The reaction solution is diluted with ice water (500 ml) and neutralized with acetic acid (150 ml). The organic layer is separated, washed with H₂O, dried over MgSO₄, filtered off and concentrated under reduced pressure. Further purification by chromatography [Petrolether/Ethyl acetate (8:2)] over a silica gel column gives the title compound (72 g) as a colorless solid. TLC, silica gel, glass plates, [Petrolether/Ethyl acetate (8:2)], R_{f}=0.47.

MS: calc: C₁₄H₁₆O₄ (248.2), fnd: [MH⁺] 249.0

### A2. 6-(2-Methyl-2,3-dihydro-benzofuran-5-yl)-2-thioxo-2,3-dihydro-1H-pyrimidin-4-one

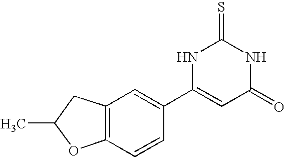

To a stirred solution of 3-(2-Methyl-2,3-dihydro-benzofuran-5-yl)-3-oxo-propionic acid ethylester (37 g, 150 mmol) and sodium ethylate (20%, 100 ml, 223 mmol) in absolute ethanol (400 ml) thiourea (22.5 g, 298 mmol) is added and the mixture is stirred under reflux for 4 d. After cooling to room temperature the reaction solution is evaporated in vacuo and the resulting residue is dissolved in aqueous 2N HCl (400 ml). The crude product is filtered and washed with HO and recrystallized in ethyl acetate and toluene to give the title compound (58 g) as a colorless solid. TLC, silica gel, glass plates, [Toluene/Acetone (9:1)], R_{f}=0.29.

MS: calc: C₁₃H₁₂N₂O₂S (260.2), fnd: [MH⁺] 261.0

### A3. 6-(2-Methyl-2,3-dihydro-benzofuran-5-yl)-3H-pyrimidin-4-one

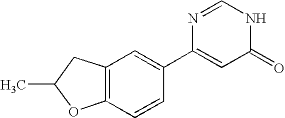

6-(2-Methyl-2,3-dihydro-benzofuran-5-yl)-2-thioxo-2,3-dihydro-1H-pyrimidin-4-one (30 g, 115 mmol) is dissolved in H₂O (300 ml). After addition of an aqueous solution of ammonia (25%, 130 ml) and Raney-Nickel WII (30 g) in H₂O (20 ml) the reaction mixture is stirred under reflux for 16 h. The reaction mixture is filtered from the solid material, which is washed again with a mixture of ammonia and H₂O (1:2, 300 ml). The combined aqueous layers are evaporated in the vacuo and coevaporated with toluene (3×) to give the title compound (31 g) as a colorless solid. TLC, silica gel, glass plates, [CH₂Cl₂/MeOH (95:5)], R_{f}=0.36.

MS: calc: C₁₃H₁₂N₂O₂ (228.2), fnd: [MH⁺] 229.0

### A4. 4-Chloro-6-(2-methyl-2,3-dihydro-benzofuran-5-yl)-pyrimidine

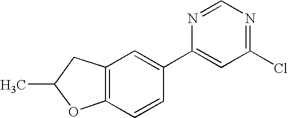

6-(2-Methyl-2,3-dihydro-benzofuran-5-yl)-3H-pyrimidin-4-one (31 g, 136 mmol) is dissolved in POCl₃ (120 ml) and stirred under reflux for 2 h. After cooling to room temperature the reaction solution is poured on ice water (1.51) and stirred for 1 h. The solution is neutralized by the addition of K₂CO₃ powder and extracted with ethyl acetate (3×500 ml). The organic phase is dried over MgSO₄, filtered off and concentrated under reduced pressure. Further purification by chromatography [Toluene/Acetone (95:5)] over a silica get column gives the title compound (24 g) as a beige solid. TLC, silica gel, glass plates [Toluene/Acetone (9:1)], R_{f}=0.43.

MS: calc: C₁₃H₁₁ClN₂O (246.7). found: [MH⁺] 247.2, 249.2

### A5. {4-[6-(2-Methyl-2,3-dihydro-benzofuran-5-yl)-pyrimidin-4-yl-amino]-phenyl}-carbamic acid tert-butyl ester

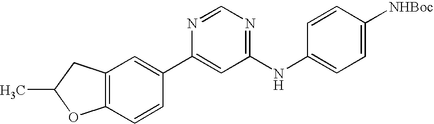

### 4-Chloro-6-(2-methyl-2,3-dihydro-benzofuran-5-yl)-pyrimidin (10 g, 41 mmol) is dissolved in a solution of Et₃N (9 ml) and absolute DMF (50 ml), (4-Amino-phenyl)-carbamic acid tert-butylester (9.3 g, 45 mmol) is added and the reaction mixture is stirred for 2 h at 140° C. The reaction solution is diluted with CH₂Cl₂ (80 ml) and extracted with a semisaturated aqueous NaCl solution (80 ml). The organic phase is dried over MgSO₄, filtered off and concentrated under reduced pressure. Further purification by chromatography [CH₂Cl₂/MeOH (95:5)] over a silica gel column gives the title compound (8.2 g) as a colorless solid. TLC, silica gel, glass plates, [CH₂Cl₂/MeOH (95:5)], R_{f}=0.35.

MS: calc: C₂₄H₂₆N₄O₃ (318.5), fnd: [MH⁺-Boc] 319.1; [MH⁺-56]363.2; [MH⁺] 419.1; [2 MH⁺] 836.3

### A6. N-[6-(2-Methyl-2,3-dihydro-benzofuran-5-yl)-pyrimidin-4-yl]-benzene-1,4-diamine dihydrochloride

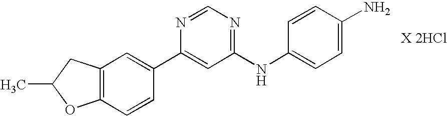

A suspension of {4-[6-(2-Methyl-2,3-dihydro-benzofuran-5-yl)-pyrimidin-4-yl-amino]-phenyl}-carbamic acid tert-butyl ester (19.6 g; 47 mmol) in dioxane (90 ml) is admixed with a saturated solution of HCl In dioxane (60 ml) and stirred at room temperature for 2 h. The reaction mixture is diluted with diethyl ether and the resulting precipitate is filtered off under an N₂ atmosphere and washed with diethyl ether (3×50 ml). Drying under reduced pressure gives the title compound (16.5 g) as a colorless solid.

MS: calc: C₁₉H₁₆N₄O (318.4), fnd: [MH⁺] 319.3

Example of the synthesis of intermediates according to step 5 and step 6 (compare reaction scheme 1):

### A7. [4-(6-Chloro-pyrimidin-4-ylamino)-phenyl]-carbamic acid tert-butyl ester

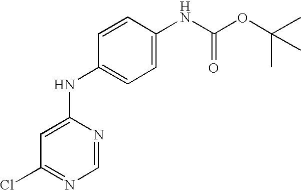

A solution of 4,6-dichloropyrimidine (53.6 g), (4-amino-phenyl)-carbamic acid tert-butyl ester (50.0 g), DABCO (2.7 g) and diisopropyl ethyl amine (46.6 g) in dimethylformamide is stirred at 120° C. under an atmosphere of nitrogen for 2 h. The solvent is removed in vacuo. The residue is dissolved in ethyl acetate and washed with 1M citric acid and saturated aqueous NaCl solution. The organic layer is dried over MgSO₄ and filtered through a plug of neutral alumina. The filtrate is concentrated in vacuo. 48.2 g of pure product is obtained after crystallization from tert-BuOMe and hexane as off-white solid.

¹H-NMR (DMSO-d₆₅) [symbol] (ppm): 9.71 (s, 1H, —NH); 9.29 (s, 1H, —NH); 8.41 (d, J=0.6 Hz, 1H); 7.44 (s, 4H); 6.69 (d, J=0.6 Hz, 1H); 1.47 (s, 9H).

MS (ESI): m/e=321.0 (MH⁺100%); 265.2 (MH⁺ −56, 62%); 221.3 (MH⁺ −100, 12%).

### A8. {4-[6-(4-fluoro-phenyl)-pyrimidin-4-ylamino]-phenyl}-carbamic acid tert-butyl ester

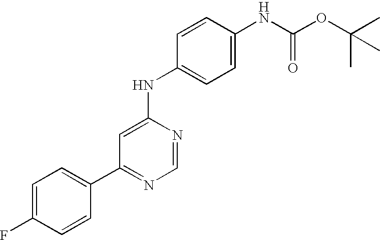

[4-(6-Chloro-pyrimidin-4-ylamino)-phenyl]-carbamic acid tert-butyl ester (7.0 g) and 4-fluorophenyl-boronic acid (4.6 g) is dissolved in degassed dioxane. After addition of 2 M aqueous Na₂CO₃ solution (33 ml) and trans-dichlorobis(tricyclohexylphosphine)palladium (0.8 g) the reaction mixture is stirred at 90° C. under an atmosphere of nitrogen for 3 h. Saturated aqueous NaCl solution is added, the organic layer is separated, dried over MgSO₄ and filtered through a plug of neutral alumina. The filtrate is concentrated in vacuo. 5.2 g of pure product is obtained after crystallization from ethyl acetate as off-white solid.

¹H-NMR (DMSO-d₈) [symbol] (ppm): 9.52 (s, 1H, —NH); 9.26 (s, 1H, —NH); 8.65 (d, J=0.7 Hz, 1H); 8.07 (m, 2H); 7.55 (d, J=9.0 Hz, 2H); 7.43 (d, J=9.0 Hz, 2H); 7.35 (d×d, J_{1′}=J₂=8.9 Hz, 2H); 7.13 (d, J=0.7 Hz, 1H); 1.48 (s, 9H).

MS (ESI): m/e=381.1 (MH⁺, 100%); 325.2 (MH⁺ −56, 59%), 281.3 (MH⁺ −100, 18%).

Example of the synthesis according to step 7 and step 8 (compare reaction scheme 1):

### A9. N-[6-(4-Fluoro-phenyl)pyrimidin-4-yl]-benzene-1,4-diamine

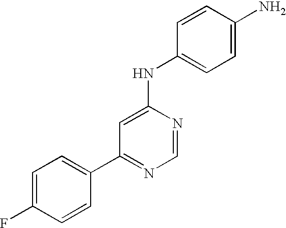

{4-[6-(4-fluoro-phenyl)-pyrimidin-4-ylamino]-phenyl}-carbamic acid tert-butyl ester (5.2 g) is dissolved in dioxane. 4N HCl in dioxane is added and the well stirred reaction mixture is heated to 50° C. for several hours. The HCl salt of the product is precipitated by addition of Et₂O. The precipitate is filtered and distributed between aqueous Na₂CO₃ and ethyl acetate. The organic layer is separated and dried over MgSO₄. The solvent is removed in vacuo. 4.9 g of pure product is obtained after crystallization from ethyl acetate and hexane as off-white solid.

¹H-NMR (DMSO-d₆) [symbol] (ppm): 9.17 (s, 1H, —NH); 8.55 (s, 1H); 8.02 (m, 2H); 752 (d×d, J1=J2= 8.8 Hz, 2H); 7.21 (d, 2H); 6.98 (s, 1H); 6.60 (d, J=8.8 Hz, 2H); 4.95 (s, 2H, —NH₂).

MS (ESI): m/e=281.3 (MH⁺, 100%).

### 1. 2,6-Difluoro-N-{4-[6-(4-fluoro-phenyl)-pyrimidin-4-ylamino]-phenyl}-benzenesulfonamide

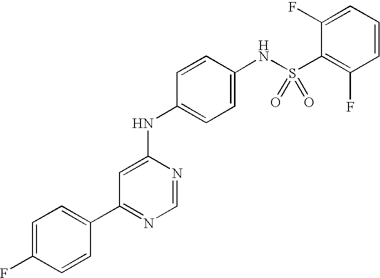

Synthesis details are described in the section Final Products.

Example of the synthesis according to step 9, step 10 and step 11:

### A10. N-(6-Chloro-pyrimidin-4-yl)-benzene-1,4-diamine

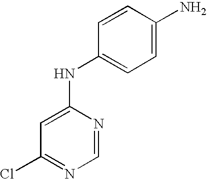

[4-(6-Chloro-pyrimidin-4-ylamino)-phenyl]-carbamic acid tert-butyl ester (160.4 g) is dissolved in dioxane and treated with 4N HCl in dioxane at 50° C. over night under an atmosphere of nitrogen. The hydrochloride of the product is precipitated with Et₂O and isolated by filtration. Precipitation from the aqueous solution of the collected hydrochloride with 2M aqueous Na₂CO₃ solution yields 110.0 g of the free base as off-white solid.

¹H-NMR (DMSO-d₆) [symbol] (ppm): 9.40 (s, 1H, —NH); 8.31 (s, 1H); 7.11 (d, J=8.3 Hz, 2H); 6.58 (s, 1H), 6.54 (d, J=8.3 Hz, 2H); 5.01 (s, 2H, —NH₂).

MS (ESI): m/e=221.2 (MH⁺, 100%).

### A11. N-[4-(6-chloro-pyrimidin-4-ylamino)-phenyl]-2,4-difluoro-benzenesulfonamide

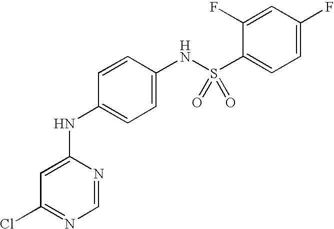

N-(6-Chloro-pyrimidin-4-yl)-benzene-1,4-diamine (11.1 g) and 2,6-lutidine (6.5 g) is dissolved in dimethoxyethane. After slow addition of 2,4-difluoro-benzenesulfonyl chloride (11.7 g) the reaction mixture is stirred under an atmosphere of nitrogen over night at ambient temperature. After addition of 1N HCl and saturated aqueous NaCl solution the organic layer is separated and concentrated in vacuo. 17.7 g of pure product is obtained after crystallization from methanol/water as off-white solid.

¹H-NMR (DMSO-d₆) [symbol] (ppm): 10.50 (s, 1H, —NH); 9.78 (s, 1H, —NH); 8.42 (s, 1H); 7.86 (d×d×d, J₁=J₂=8.6 Hz, J₃= 6.4 Hz); 7.52 (d, J=8.8 Hz, 2H, and m, 1H); 754 (d×d×d, J₁=J₂=8.6 Hz, J₃=1.7 Hz); 7.09 (d, J=8.8 Hz, 2H); 6.73 (s, 1H).

MS (ESI): m/e=397.0 (MH⁺, 100%).

### 2. 2,4-Difluoro-N-(4-{6-[4-(1-methyl-piperidin-4-ylmethoxy)-phenyl]-pyrimidin-4-ylamino}-phenyl)-benzenesulfonamide

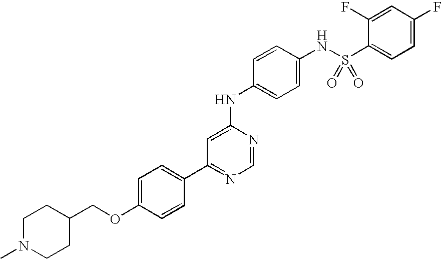

Synthesis details are described in the section Final Products.

Example for the synthesis of a boronic acid ester of formula 9b —Reaction scheme 2:

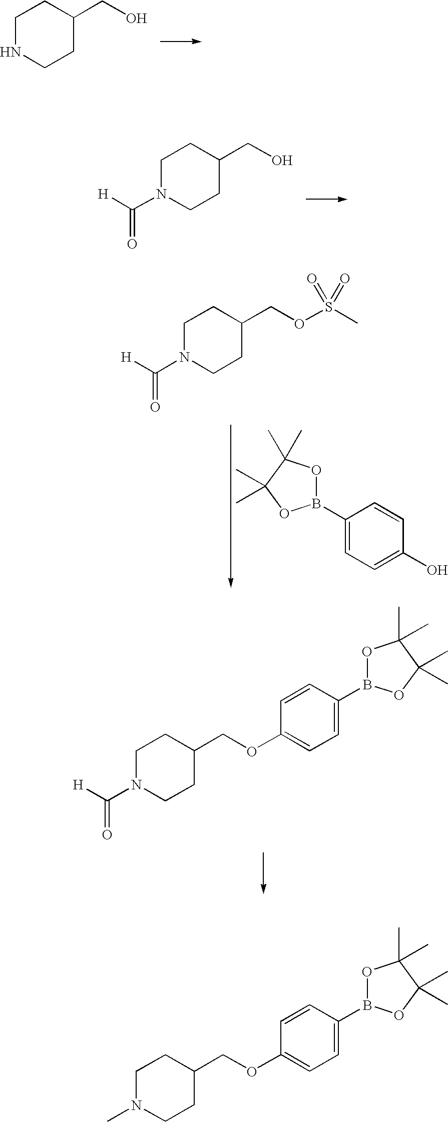

### A12. Methanesulfonic acid 1-formyl-piperidin-4-ylmethyl ester

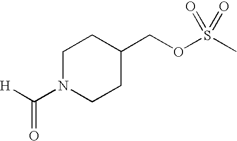

To a well stirred solution of piperidin-4-yl-methanol (69.0 g) in dichloromethane methyl formate (46.8 g) is added with care. The reaction is stirred at ambient temperature for 6 h. The solvent is completely removed In vacuo. The residual pale yellow oil is redissolved in dichloromethane and triethylamine (106.5 g) is added to the well stirred solution. A solution of methanesulfonyl chloride (82.5 g) is added dropwise at 0° C. and after complete addition the reaction mixture is stirred at ambient temperature for additional 4 h. The organic layer is extracted with 0.5 N aqueous HCl and dried over MgSO₄. The solvent is removed in vacuo. Pure product 69.9 g is obtained as off-white solid after crystallization from cyclohexane.

MS: calc: C₈H₁₅NO₄S (221.28) fnd: [MH⁺]: 222.1

### A13. 4-[4-(4,4,5,5-Tetramethyl-[1.3.2]dioxaborolan-2-yl)-phenoxymethyl]-piperidine-1-carbaldehyde

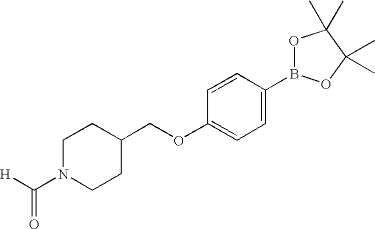

Sodium hydride (60% dispersion in oil, 1.1 g) is washed with hexane and suspended in dry dimethylformamide. 4-(4,4,5,5-Tetramethyl-[1,3,2]dioxaborolan-2-yl)-phenol (5.2 g) is added in small portions to the well stirred suspension under cooling to 0° C. and stirring is continued under an atmosphere of nitrogen for 1 h. Methanesulfonic acid 1-formyl-piperidin-4-ylmethyl ester (6.1 g) is added and the reaction mixture is heated to 100° C. for 1 h. After cooling to ambient temperature the reaction is quenched with water and the product is extracted into ethyl acetate. After drying over MgSO₄ the solvent is removed in vacuo. The residue is crystallized from acetonitrile to yield 4.4 g of pure product as an off-white solid.

¹H-NMR (CDCl₃) [symbol] (ppm): 8.0 (s, 1H); 7.7 (d, 2H); 6.8 (d, 2H); 4.5 (m, 1H); 3.8 (t, 2H); 3.6 (m, 1H); 2.0 (m, 2H+1H); 1.3 (s, 12H); 1.2 (m, 2H).

### A14. 1-Methyl-4-[4-(4,4,5,5-tetramethyl-[1.3.2]dioxaborolan-2-yl)-phenoxymethyl]-piperidine

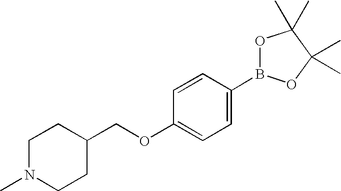

Borane (1M solution in tetrahydrofurane, 25.0 ml) is dropwise added to a solution of 4-[4-(4,4,5,5-Tetramethyl-[1,3,2]dioxaborolan-2-yl)-phenoxymethyl]-piperidine-1-carbaldehyde (4.3 g) in tetrahydrofurane, then the stirred mixture is heated to gentle reflux for 1.5 h under an atmosphere of nitrogen. After addition of further borane solution (2.5 ml) the mixture is refluxed for another 5 h before addition of pinacol (5.9 g) and again refluxing for 2 h. The volatile materials are removed in vacuo. The residue is purified by Kugel-rohr distillation to deliver 1.5 g of the pure product (bp. 160° C., 0.1 mbar) after a forerun of pinacol as colourless oil, which solidified on standing.

¹H-NMR (CDCl₃) [symbol] (ppm): 7.7 (d, 2H); 6.8 (d, 2H); 3.7 (t, 2H); 2.8 (m, 2H); 2.2 (s, 3H); 1.7 (m, 2H+ 2H+1H); 1.4 (m, 2H); 1.3 (s, 12H).

Additional example for the synthesis of a boronic acid ester of formula 9b—reaction scheme 3:

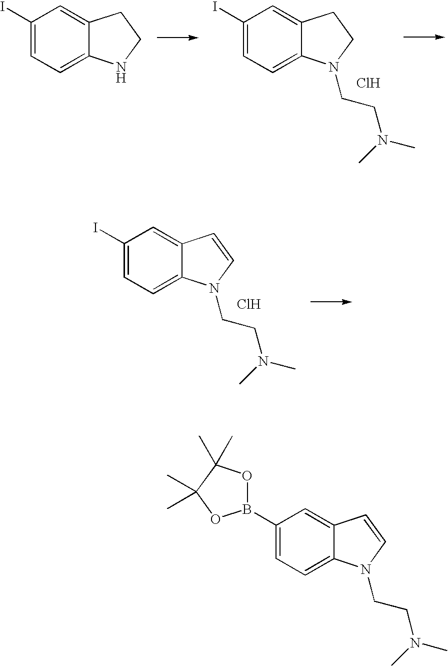

### A15. [2-(5-Iodo-indol-1-yl)-ethyl]-dimethyl-amine hydrochloride

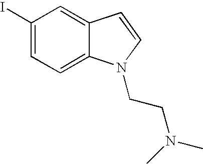

Oil free sodium hydride (prepared from 4.0 g 60% dispersion in oil by washing with hexane) is suspended in DME, DMSO (9:1). To the well-stirred suspension 5-Iodo-1H-indole (9.72 g) is added in portions. After stirring for 30 min (2-chloro-ethyl)-dimethyl-amine hydrochloride (6.91 g) is added and the reaction mixture is stirred at 70° C. for 16 h under an atmosphere of nitrogen. The reaction mixture is quenched by slow addition of ice-cold water. After saturation of the aqueous layer with solid NaCl the organic layer is separated and concentrated in vacuo. The aqueous layer is extracted with AcOEt. All aqueous layers are combined, washed with brine and dried over MgSO₄. After evaporation the crude product is chromatographed on neutral alumina (act. 2-3) using AcOEt as eluent. The title compound (12.54 g) is obtained as a pale yellow oil.

MS: calc: C₁₂H₁₅IN₂ (314.17) fnd: [MH⁺]: 315.1

The hydrochloride is obtained as an off-white solid by dissolving the free base in Et₂O and slow addition of a small excess of 4N HCl in dioxane.

### A16. Dimethyl-{2-[5-(4,4,5,5-tetramethyl-[1,3,2]dioxaborolan-2-yl)-indol-1-yl]-ethyl}-amine

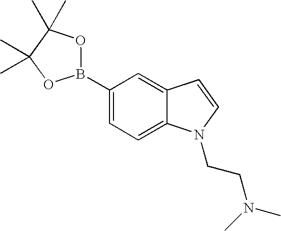

[2-(5-Iodo-indo-1-yl)-ethyl]-dimethyl-amine hydrochloride (10.0 g) is suspended in dry dioxane. After addition of dry Et₃N (14.42 g), PdCl₂ (dppf)×CH₂Cl₂ (0.35 g) and 4,4,5,5-Tetramethyl-[1,3,2]dioxaborolane (5.47 g) the reaction mixture is stirred at 80° C. for about 1 h under an atmosphere of nitrogen. After addition of ice cold water the reaction mixture is concentrated in vacuo. The residue is extracted with several portions AcOEt. The combined organic layers are dried over MgSO₄. After filtration the solvent is removed in vacuo. The crude product is filtered through a plug of neutral alumina (act. 2-3) using AcOEt as eluent and further purified by bulb to bulb distillation (200° C.-220° C., 1.8×10⁻² mbar). The title compound (8.90 g) is obtained as a pale yellow oil.

MS: calc: C₁₈H₂₇BN₂O₂ (314.24) fnd: [MH⁺]: 315.2

¹H-NMR (DMSO-d₆) δ (ppm): 7.94 (s, 1H); 7.45 (s, 2H); 7.37 (d, J=3.2 Hz, 1H); 6.46 (d, J=3.2 Hz, 1H); 455 (t, J=6.6 Hz, 2H); 2.60 (t, J=6.6 Hz, 2H); 2.18 (s, 6H); 1.31 (s, 12H).

### Biological Assays for the Investigation of T Cell Associated Kinase Inhibitors

The compounds of formula 1 in free form or in pharmaceutically acceptable salt form exhibit valuable pharmacological properties, e.g. inhibition of protein kinases like p90 ribosomal S6 kinase (Rsk) family, Src family kinases, e.g. Lck or Protein Kinase C (PKC), e.g. PKC isoforms like a or 6 activity, inhibition of T lymphocyte activation and proliferation, e.g. by inhibiting production of cytokines by T lymphocytes, e.g. IL-2, by inhibiting the proliferative response of T lymphocytes to cytokines, e.g. IL-2, as indicated in in vitro tests and are therefore indicated for therapy.

### A. Biochemical Tests

### 1. Protein Kinase C Assay

The compounds of formula 1 are tested for their activity on different protein kinases and PKC isoforms using a scintillation proximity assay (SPA, Amersham International plc). ³³P-labeled peptides are captured onto streptavidin coated yttrium silicate SPA beads. β-particles, emitted from the captured ³³P-labeled substrate in close proximity to the bead are able to excite the scintillant, resulting in the generation of quantifiable light. The assay is performed in a 96-well polystyrene microtiterplate (1450-514, Isoplates, Wallac, Turku, Finland). The reaction mixture (50 μl) contains 10 μl of the test compound together with 10 μl of the relevant kinase, diluted in the relevant dilution buffer, 5 μl of 10 μM phorbol myristate acetate (PMA) in H₂O, 5 μl of 1.6 mM phosphatidylserine (Sigma-Aldrich Chemie GmbH, Stein-heim, Germany) in 20 mM Tris/HCL buffer pH 7.4, 5 μl of 0.3% BSA in H₂O, 5 μl of 30 μM relevant substrate and 10 μl of 5 μM ATP and 0.1 μCi of ³³P-ATP (Amersham, Freiburg, Germany) in 200 mM Tris/HCL pH 7.5 and 200 mM MgCl₂. Incubation is performed for 40 min at room temperature (RT) without shaking. Reaction is stopped by adding 150 μl of cold stop solution containing 10 mM ATP, 5 mM EGTA pH 7.5, 0.1% Triton X-100 and 0.2 mg streptavidin coated yttrium silicate SPA beads (Amersham, RPNQ 0012). The sealed plate is incubated for 60 min at RT. Thereafter the MTP is counted in a Microbeta Jet (Wallac). IC₅₀ measurement is performed on a routine basis by incubation a serial dilution of inhibitor at concentrations ranging between 0.01 and 100 μM according to the method described above. Background values are the signals of the reaction mixture without addition of the relevant kinase and are subtracted from all values. 100% values are the signals of the reaction mixture without addition of inhibitors.

Dependent on the efficacy of the inhibitors In the various test systems, corresponding IC₅₀ values are calculated from concentration-inhibition curves by nonlinear regression analysis using the program GraphPad Prism (GraphPad Software Inc., San Diego, Calif.).

### 2. Protein Kinase CαAssay

Human recombinant PKCα was obtained from Panvera (Invitrogen GmbH, Karlsruhe, Germany) and is used under the assay conditions as described above (Section A.1). The enzyme is diluted in PKC dilution buffer containing 1 mM Hepes pH 7.4, 0.5 mM DTT and 0.001% Triton X-100. The assay additionally contains 0.5 mM CaCl₂. The kinase reaction is performed with the biotinylated PKCα pseudosubstrate solved in H₂O. The examples 16, 37, 119, 120 and 150 Inhibit PKCα in this assay with an IC₅₀ between 1 and 18 μM.

### 3. Protein Kinase Cβ1 Assay

Human recombinant PKCβ1 was obtained from Panvera and is used under the assay conditions as described above (Section A.1). The enzyme is diluted in PKC dilution buffer containing 1 mM Hepes pH 7.4, 0.5 mM DTT and 0.001% Triton X-100. The assay additionally contains 0.5 mM CaCl₂. The kinase reaction is performed with the biotinylated PKCα pseudosubstrate solved in H₂O. The examples 29, 45, 52, 128 and 139 inhibit PKC β1 in this assay with an IC₅₀ between 2 and 40 μM.

### 4. Protein Kinase C δ Assay

Human recombinant PKCδ was obtained from Panvera and is used under the assay conditions as described above (Section A.1). The enzyme is diluted in PKC dilution buffer containing 1 mM Hepes pH 7.4, 0.5 mM DTT and 0.001% Triton X-100. The kinase reaction is performed with the biotinylated PKOη pseudosubstrate solved in H₂O. The examples 122, 132, 138 and 168 inhibit PKC Sin this assay with an IC₅₀ between 2 and 18 μM.

### 5. Protein Kinase C ε Assay

Human recombinant PKCε was obtained from Panvera and is used under the assay conditions as described above (Section A.1). The enzyme is diluted in PKC dilution buffer containing 1 mM Hepes pH 7.4, 0.5 mM DTT and 0.001% Triton X-100. The kinase reaction is performed with the biotinylated PKCη pseudosubstrate solved in H₂O. The examples 27, 38,47 and 117 inhibit PKC ε in this assay with an IC₅₀ between 4 and 21 μM

### 6. Protein Kinase C η Assay

Human recombinant nickel bead purified PKCη from Sf21 insect cells is used under the assay conditions as described above (Section A.1). The enzyme is diluted in PKC dilution buffer containing 1 mM Hepes pH 7.4, 0.5 mM DTT and 0.001% Triton X-100. The kinase reaction is performed with the biotinylated PKCη pseudosubstrate solved in H₂O. The examples 52, 55, 118 and 135 inhibit PKC η in this assay with an IC₅₀ between 6 and 50 μM.

### 7. Protein Kinase C θ Assay

Human recombinant nickel bead purified PKCθ from Sf21 insect cells is used under the assay conditions as described above (Section A.1). The enzyme is diluted in PKC dilution buffer containing 1 mM Hepes pH 7.4, 0.5 mM DTT and 0.001% Triton X-100. The kinase reaction is performed with the biotinylated PKCθ pseudosubstrate solved in H₂O. The examples 8, 22, 46, 115, 118 and 161 inhibit PKC 9 in this assay with an IC₅₀<1 μM.

### 8. Protein Kinase C ι Assay

Human recombinant nickel bead purified PKCι from Sf21 insect cells is used under the assay conditions as described above (Section A.1) but in the absence of PMA and phosphatidylserine. The enzyme is diluted in PKC dilution buffer containing 1 mM Hepes pH 7.4, 0.5 mM DTT and 0.001% Triton X-100. The kinase reaction is performed with the biotinylated PKCι/ζ pseudosubstrate solved in H₂O. The examples 145, 162 and 173 inhibit PKC ι in this assay with an IC₅₀ between 7 and 34 μM.

### 9. Protein Kinase C ζ Assay

Human recombinant PKCζ was obtained from Panvera and is used under the assay conditions as described above (Section A.1) but in the absence of PMA and phosphatidylserine. The enzyme is diluted in PKC dilution buffer containing 1 mM Hepes pH 7.4, 0.5 mM DTT and 0.001% Triton X-100. The kinase reaction is performed with the biotinylated PKCι/ζ pseudosubstrate solved in HA The examples 143 and 175 inhibit PKC C, in this assay with an IC₅₀ between 15 and 25 μM.

### 10. Protein Kinase A Assay

Human recombinant PKA was obtained from Panvera and is used in a scintillation proximity assay described in Section A.1. The reaction mixture (50 μl) contains 15 μl of the test compound together with 10 μl of PKA in 1 mM Hepes pH 7.4, 0.001% Triton X-100 and 1 mM DTT, 5 μl of 03% BSA in HA. 10 μl of 15 μM biotinylated PKA substrate peptide (Upstate) and 10 μl of 5 μM ATP and 0.1 μCi of ³²P-ATP in 200 mM Tris/HCL pH 7.5 and 100 mM MgCl₂. Incubation is performed for 40 min at room temperature (RT) without shaking. Reaction is stopped by adding 150 μl of cold stop solution containing 10 mM ATP, 5 mM EGTA pH 7.5, 0.1% Triton X-100 and 0.2 mg streptavidine coated yttrium silicate SPA beads (Amersham, RPNQ 0012). The sealed plate is incubated for 60 min at RT. Thereafter the MTP is counted in a Microbeta Jet (Wallac). IC₅₀ measurement is performed on a routine basis by incubation a serial dilution of inhibitor at concentrations ranging between 0.01 and 100 μM according to the method described above. Background values are the signals of the reaction mixture without addition of the relevant kinase and are subtracted from all values. 100% values are the signals of the reaction mixture without addition of inhibitors. IC₅₀ values are calculated from the graph by sigmoidal curve fitting. The examples 10, 17, 117 and 121 inhibit PKA in this assay with an IC₅₀ between 3 and 16 μM.

11. Lck (p56^{Lck}) Assay

Human recombinant Lck was obtained from Upstate (Dundee, UK) and is used in a scintillation proximity assay described in Section A.1. The reaction mixture (50 μl) contains 15 μl of the test compound together with 10 μl of Lck in 1 mM Hepes pH 7.4, 0.001% Triton X-100 and 1 mM DTT, 5 μl of 0.3% BSA in H₂O, 10 μl of 150 μM biotinylated synthetic peptide obtained from Biotrend (Köln, Germany) and 10 μl of 5 μM ATP and 0.1 μCi of ³³P-ATP in 200 mM Tris/HCL pH 7.5 and 100 mM MgCl₂. Incubation is performed for 40 min at room temperature (RT) without shaking. Reaction is stopped by adding 150 μl of cold stop solution containing 10 mM ATP, 5 mM EGTA pH 7.5, 0.1% Triton X-100 and 0.2 mg streptavidine coated yttrium silicate SPA beads (Amersham, RPNQ 0012). The sealed plate is incubated for 60 min at RT. Thereafter the MTP is counted in a Microbeta Jet (Wallac). IC50 measurement is performed on a routine basis by incubation a serial dilution of inhibitor at concentrations ranging between 0.01 and 100 μM according to the method described above. Background values are the signals of the reaction mixture without addition of the relevant kinase and are subtracted from all values. 100% values are the signals of the reaction mixture without addition of inhibitors. IC₅₀ values are calculated from the graph by sigmoidal curve fitting. The examples 128 and 131 inhibit Lck in this assay with an IC₅₀<3 μM

### 12. Rsk1, Rsk2, Rsk3 Assay

To investigate the effect of a test compound on p90 ribosomal S6 kinase (Rsk) family, the compounds are externally tested in KinaseProfiler assay (Upstate ltd, Dundee, UK) described in product guide brochure. In brief, in a final reaction volume of 25 μl, 5-10 mU of human recombinant Rsk are incubated with 8 mM MOPS pH 7.0, 0.2 mM EDTA, 30 μM KKKNRTLSVA, 10 mM MgAcetate and 10 μM [γ-33P-ATP]. The reaction is initiated by addition of the MgATP mix. After incubation for 40 minutes at RT, the reaction is stopped by the addition of 5 μl of a 3% phosphoric acid solution. 10 μl of the reaction is then spotted onto P30 filtermat and washed three times for 5 minutes in 75 mM phosphoric acid and once in methanol prior to drying and scintillation counting. The IC₅₀ value is estimated by preparing a 10 point curve using a ½ log dilution series (IC₅₀Profiler, Upstate). The examples 129, 431 and 173 inhibit Rsk1 in this assay with an IC₅₀<1 μM.

### B Cellular Tests

### 1. CD3/CD28 Costimulation Assay

The assay is performed with freshly isolated primary human CD4+ T lymphocytes. CD4+ T lymphocytes from whole blood are prepared using negative selection as previously described (Hatzelmann and Schudt, J. Pharmacol. Exp. Ther. 2001; 297:267-279). In brief, peripheral blood mononuclear cells (PBMC) are isolated by density gradient centrifugation using a Percoll gradient (p= 1.077 g/ml). 1×10⁷ PBMC were then resuspended in 30 μl of PBS containing 0.5% FCS and human CD4+ T lymphocytes are isolated by depletion of non CD4+ T cells. For this, non-CD4+ T cells are Indirectly magnetically labeled with 10 μl of a cocktail of biotin-conjugated monoclonal antibodies (against CD8, CD14, CD16, CD19, CD36, CD56, CD123, TCR γ/δ; MACS CD4+ T cell isolation kit II, Miltenyi Biotec), as primary labeling reagent, and anti-biotin monoclonal antibodies conjugated to MicroBeads, as secondary labeling reagent The magnetically labeled non-CD4+ T cells are depleted by retaining them on a MACS column in the magnetic field of a MACS separator (Miltenyi Biotec, Bergisch Gladbach, Germany) according to the manufacturer's instruction. CD4+ T cells are resuspended in RPM 11640 containing 10% heat-inactivated FCS, 2 mM L-glutamine, 100 U/ml penicillin and 100 μg/ml streptomycin (Gibco Life Technologies).

2×10⁵ CD4+ T cells in a total assay volume of 200 μl are stimulated via the T-cell receptor and the costimulatory molecule CO₂₈ by using corresponding selective mAbs as previously described (Hatzelmann and Schudt, J. Pharmacol. Exp. Ther. 2001; 297:267-279). The assay is performed in 96 well tissue culture plates (655180, flat bottom, Greiner, Frickenhausen, Germany). Cells were maintained at 37° C. in a humidified atmosphere of 5% CO₂ in an incubator (type BB6220 CU, Heraeus Instruments, Hanau, Germany). For determination of IL-2 level, all assays are performed in duplicate, and after 48 h of growth, supernatants are removed, pooled in 96 well plates (650101, U-shape, Greiner) and stored at −20° C. before measurement of IL-2 with a commercially available enzymimmunoassay kit from Coulter-Immunotech Diagnostics (Marseille, France) according to the manufacturer's instruction. For each experiment the appropriate dilution factor for IL-2 is determined. Dilutions are performed in diluent D (Coulter-Immunotech Diagnostics) and IL-2 for one condition is determined from the pool fraction in duplicate in a ELISA-reader (Rainbow, Tecan, Crailsheim, Germany) at 450 nm.

To investigate the effect of a test compound on the IL-2 release of stimulated CD4+ T cells, six three-fold dilution steps in duplicates per test compound are performed (final DMSO concentration 0.1%). Low control values are the signals from non stimulated CD4+ T cells; high controls are the signals from stimulated CD4+ T cells without any test samples. Low controls are subtracted from all values. The inhibition obtained In the presence of a test compound is calculated as percent inhibition of the high control. The concentration of test compounds resulting in 50% inhibition (IC₅₀) is determined from the dose-response curves. The examples 13, 19, 20 and 136 inhibit IL-2 release from stimulated CD4+ T lymphocytes in this assay with an IC₅₃ between 1 and 9 μM.

### 2. Mixed Lymphocyte Reaction (MLR)

For MLR cultures 4×10⁵ responder T cells are incubated in duplicates with 2×10⁵ mitomycin C-treated allogeneic stimulator T cells in a total volume of 200 μl RPMI 1640 medium supplemented with 10% FCS, 2 mM L-glutamine, 100 U/ml penicillin and 100 μg/ml streptomycin (Gibco Life Technologies) in 96 well tissue culture plates (655180, flat bottom, Greiner). Cells are maintained at 37° C. in a humidified atmosphere of 5% CO₂. After 48 h of growth, cells are pulsed with 1 μCi [³H]thymidine and incorporation of [³H]thymidine is measured with a Topcount radioactive counter (Packard). To investigate the effect of a test compound on MLR-mediated T cell proliferation six three-fold dilution steps in duplicates per test compound are performed (final DMSO concentration 0.1%). Low control values are the proliferation of responder cells alone; high controls are from mixed lymphocyte cells without any test samples. Low controls are subtracted from all values. The inhibition obtained in the presence of a test compound is calculated as percent Inhibition of the high control. The concentration of test compounds resulting in 50% inhibition (IC₅₀) is determined from the dose-response curves. The examples 91, 128, 133 and 137 inhibit MLR-mediated T-cell proliferation in this assay with an IC₅₀<1 μM.

The compounds according to the invention are, therefore, useful in the treatment and/or prevention of diseases or disorders mediated by T lymphocytes and/or PKC, e.g. acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock. The compounds according to the invention are also useful in the treatment and/or prevention of T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

The invention further relates to a method for the treatment of mammals, including humans, which are suffering from one of the abovementioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

The pharmaceutical compositions are prepared by processes, which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral and intravenous delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the active compounds is carried out In the order of magnitude customary for kinase Inhibitors. Topical application forms (such as ointments) for the treatment of dermatoses thus contain the active compounds in a concentration of, for example, 0.1-99%. The dose for administration by inhalation is customarily between 0.1 and 10 mg per day. The customary dose in the case of systemic therapy (p.o.) is between 0.3 and 30 mg/kg per day, (i.v.) is between 0.3 and 30 mg/kg/h.

1. Compounds of formula 1 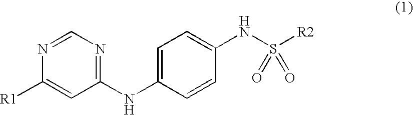
in which
R1 is phenyl,
phenyl substituted by R3 and/or R4,
naphthalenyl,
naphthalenyl substituted by R5 and/or R6,
aryl1,
aryl1 substituted by R7 and/or R8,
R9,
R10 or
R11,
R2 is phenyl,
phenyl substituted by R12 and/or R13,
naphthalenyl,
naphthalenyl substituted by R14 and/or R15,
aryl2,
aryl2 substituted by R16 and/or R17
or a radical selected from 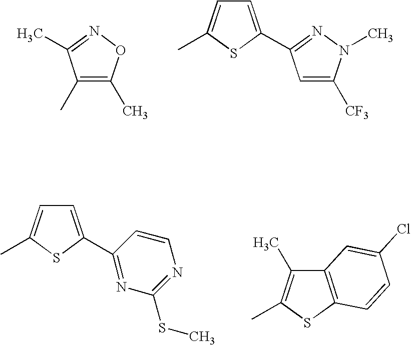
R3 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1 C-alkyl. 1-(1-4C-alkyl)-azepan-3-yl-1-4C-alkyl, 1-<1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32 wherein
R31 is hydrogen, 1-4C-alkyl or 1-4C-alkoxy-2-4C-alkyl, and
R32 is hydrogen, 1-4C-alkyl or 1-4C-alkoxy-2-4C-alkyl or wherein
R31 and R32 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
n is an integer from 0 to 4,
m is an integer from 2 to 4,
R4 is halogen, cyano, nitro, 1-4C-alkyl, trifluoromethyl, 1C-alkoxy or 1-4C-alkoxy completely or predominantly substituted by fluorine, amino or mono- or di-1-4C-alkylamino,
R5 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)piperid-2-yl-1-4C-alkyl, 1-(1-4C-alkyl)-piperid-3-yl-1-(1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl. 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R6 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
Aryl1 furanyl, thiophenyl, oxazolyl, isoxazolyl, thiazolyl, Isothiazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl or dibenzofuranyl,
R7 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R8 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R9 is unsubstituted pyrrolyl, pyrazolyl, imidazolyl, indolyl, indazolyl, benzimidazolyl or benztriazolyl, or a radical selected from 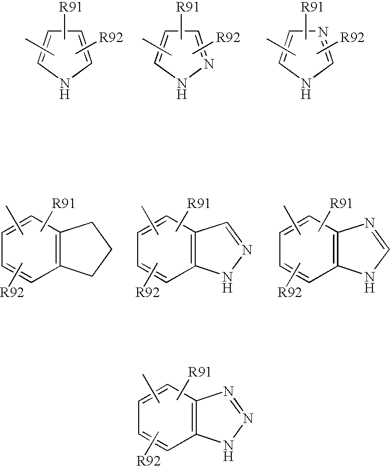
wherein
R91 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl 1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R92 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R10 is a radical selected from 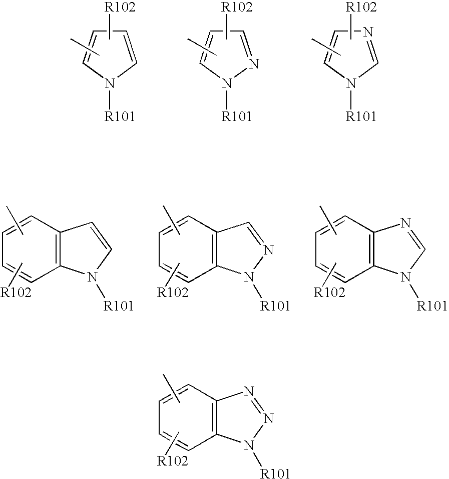
wherein
R101 is 1-4C-alkyl, 2,2,2-trifluoroethyl or 3,3,3-trifluoropropyl,
R102 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R11 is a radical selected from 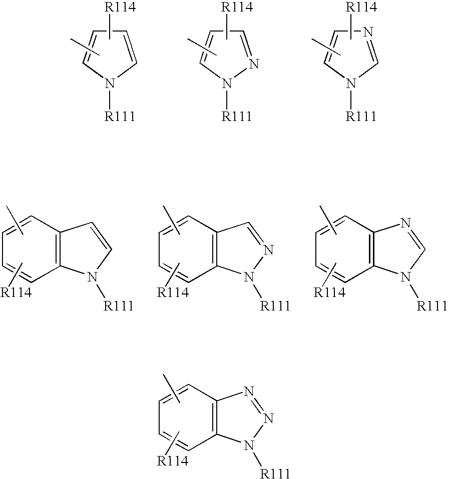
wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1C-alkyl, 1-(1-4C-alkyl)-azepan-4-yl-1-4C-alkyl, —(CH₂)ₚ—N(R112)R113 or —CH₂CH(OH)CH₂N(R112)R113, wherein
R112 is hydrogen, 1-4C-alkyl or 1-4C-alkoxy-2-4C-alkyl, and
R113 is hydrogen, 1-4C-alkyl or 1-4C-alkoxy-2-4C-alkyl, or wherein
R112 and R113 together and with inclusion of the nitrogen atom to which they are bonded form a pyrrolidine piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4&alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, Isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or dl-1-4C-alkylamino,
and the salts of these compounds with the proviso that the following compounds are excluded
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide.

2. Compounds of formula 1 according to claim 1 in which
R1 is phenyl,
phenyl substituted by R3 and/or R4,
naphthalenyl or
naphthalenyl substituted by R5 and/or R6,
R2 is phenyl,
phenyl substituted by R12 and/or R13,
naphthalenyl,
naphthalenyl substituted by R14 and/or R15,
aryl2,
aryl2 substituted by R16 and/or R17
or a radical selected from 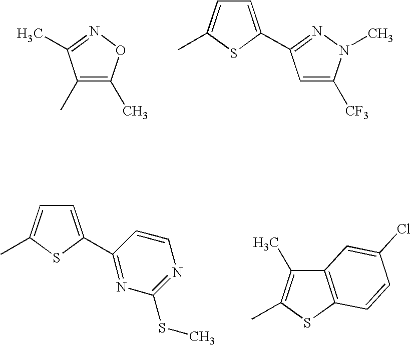
R3 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy or benzyloxy,
R4 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R5 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-4C-alkylaminocarbonyl, 1-4C-alkylcarbonylamino, phenoxy or benzyloxy,
R6 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, Isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds with the proviso that the following compounds are excluded
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidine-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide, and
N-{4-[6-(3-Dimethylamino-phenyl)-pyrimidin-4-ylamino]-phenyl}-4-methyl-benzenesulfonamide.

3. Compounds of formula 1 according to claim 1 in which
R1 is phenyl, 3-fluorophenyl, 4-fluorophenyl, 3-methoxyphenyl, 4-methoxyphenyl, 3,4-dimethoxyphenyl, 3-acetylphenyl, 4-acetylphenyl, 3-cyanophenyl, 4-phenoxyphenyl or naphthalen-1-yl,
R2 is phenyl, phenyl substituted by R12 and/or R13, thiophenyl, naphthalenyl or 5-(1-methyl-5-trifluoromethyl-1H-pyrazol-3-yl)-thiophene-2-yl,
R12 is halogen, cyano, nitro, 1-4C-alkyl, trifluoromethyl, 1-4C-alkoxy, 1-4C-alkoxy which is completely or predominantly substituted by fluorine or 1-4C-alkoxycarbonyl,
R13 is halogen, 1-4C-alkyl or 1-4C-alkoxy,
and the salts of these compounds with the proviso that the following compounds are excluded
4-Methyl-N-[4-(6-phenyl-pyrimidine-4-ylamino)-phenyl]-benzenesulfonamide,
4-Methyl-N-[4-(6-naphthalen-1-yl-pyrimidin-4-ylamino)-phenyl]-benzenesulfonamide, and
N-{4-[6-(3,4-Dimethoxy-phenyl)-pyrimidin-4-ylamino]-phenyl}-4-methyl-benzenesulfonamide.

4. Compounds of formula 1 according to claim 1 in which
R1 is phenyl, 3-fluorophenyl, 4-fluorophenyl, 3-methoxyphenyl, 4-methoxyphenyl, 4-acetylphenyl or naphthalen-1-yl,
R2 is 2-fluorophenyl, 3-fluorophenyl, 3,4-difluorophenyl, 2,4-difluorophenyl, 2,6-difluorophenyl, 3,4-dichlorophenyl, 3-chloro-4-fluorophenyl, 3-chloro-4-methylphenyl, 3-bromophenyl, 3-methylphenyl, 4-methylphenyl, 4-isopropylphenyl, 4-trifluoromethylphenyl, 4-methoxyphenyl, 4-cyanophenyl or 5-(1-methyl-5-trifluoromethyl-1H-pyrazol-3-yl)-thiophene-2-yl,
and the salts of these compounds with the proviso that the following compounds are excluded
4-Methyl-N-[4-(6-phenyl-pyrimidin-4-ylamino)-phenyl]-benzenesulfonamide and
4-Methyl-N-[4-(6-naphthalen-1-yl-pyrimidin-4-ylamino)-phenyl]-benzenesulfonamide.

5. Compounds of formula 1 according to claim 1 in which
R1 is phenyl substituted by R3 and/or R4 or naphthalenyl substituted by R5 and/or R6,
R2 is phenyl,
phenyl substituted by R12 and/or R13,
naphthalenyl,
naphthalenyl substituted by R14 and/or R15,
aryl2,
aryl2 substituted by R16 and/or R17
or a radical selected from 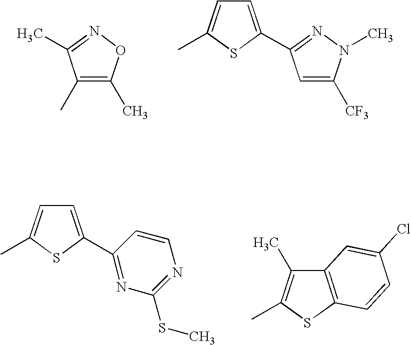
R3 is 1-(1-4C-alkyl)-pyrrolidin-2-yl-1-4C-alkyl, 1-(1-4C-alkyl)-pyrrolidin-3-yl-1C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperidin-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
R31 is hydrogen, 1-4C-alkyl or 1-4C-alkoxy-2-4C-alkyl, and
R32 is hydrogen, 1-4C-alkyl or 1-4C-alkoxy-2-4C-alkyl or wherein
R31 and R32 together and with inclusion of the nitrogen atom to which they are bonded form a pyrrolidine piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
n is an integer from 0 to 4,
m is an integer from 2 to 4,
R4 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R5 is 1-(1-4C-alkyl)-pyrrolidin-2-yl-1C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(14C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R6 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which Is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, Imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

6. Compounds of formula 1 according to claim 1 in which
R1 is phenyl substituted in para or meta position by R3 or phenyl substituted in para and meta
position by R3 and R4,
R2 is phenyl substituted by R12 and/or R13,
R3 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
R31 is hydrogen, 1-4C-alkyl or 1-4C-alkoxy-2-4C-alkyl, and
R32 is hydrogen, 1-4C-alkyl or 1-4C-alkoxy-2-4C-alkyl or wherein
R31 and R32 together and with inclusion of the nitrogen atom to which they are bonded form a pyrrolidine piperidin-, 4-hydroxy-piperidin-, piperazin-, 4-(1-4C-alkyl)piperazin-, [1,4]diazepan, 4-(1-4C-alkyl)-[1,4]diazepan-, morpholin-, thiomorpholin- or an azepan-ring,
n is an integer from 1 to 4,
m is an integer from 2 to 4,
R4 is fluorine, methyl or methoxy,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

7. Compounds of formula 1 according to claim 1 in which
R1 is phenyl substituted in para or meta position by R3,
R2 is phenyl substituted by R12 and/or R13,
R3 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-allyl)-piperid-3-yl-1-4C-alkyl, 1-(1-4C-alkyl)-piperid-4-yl-1-4C-alkyl. 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy; 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
R31 is hydrogen, 1-4C-alkyl or 1-4C-alkoxy-2-4C-alkyl, and
R32 is hydrogen, 1-4C-alkyl or 1-4C-alkoxy-2-4C-alkyl or wherein
R31 and R32 together and with inclusion of the nitrogen atom to which they are bonded form a pyrrolidine piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
n is an integer from 1 to 4,
m is an integer from 2 to 4,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

8. Compounds of formula 1 according to claim 1 in which
R1 is phenyl substituted in para position by R3 or phenyl substituted in para position by R3 and in meta position by R4,
R2 is phenyl substituted by R12 and/or R13,
R3 is morpholin-4-ylmethyl, morpholin-4-ylethyl, morpholin-4-ylpropyl, 2-morpholin-4-ylethoxy, 3-morpholin-4-ylpropoxy, 4-methylpiperazin-1-ylmethyl, 4-methylpiperazin-1-ylethyl, 4-methylpiperazin-1-ylpropyl, 2-(4-methylpiperazin-1-yl)ethoxy, 3-(4-methylpiperazin-1-yl)propoxy, 1-methylpiperidin-4-ylmethoxy, 1-methylpiperidin-4-ylmethyl, 1-methylpiperidin-4-ylethyl, 1-methylpiperidin-4-ylpropyl, 2-(1-methylpiperidin-4-yl)ethoxy, 3-(1-methylpiperidin-4-yl)propoxy, pyrrolidin-1-ylpropyl, pyrrolidin-1-ylethyl, pyrrolidin-1-ylmethyl, 3-pyrrolidin-1-ylpropoxy, 2-pyrrolidin-1-ylethoxy, piperidin-1-ylmethyl, piperidin-1-ylethyl, piperidin-1-ylpropyl, 2-(piperidin-1-yl)ethoxy, 3-(piperidin-1-yl)propoxy,
R4 is fluorine, methyl or methoxy,
R12 is fluorine, chlorine, cyano, methyl, isopropyl, trifluoromethyl or methoxy,
R13 is fluorine or chlorine,
and the salts of these compounds.

9. Compounds of formula 1 according to claim 1 in which
R1 is phenyl substituted in para or meta position by R3,
R2 is phenyl substituted by R12 and/or R13,
R3 is morpholin-4-ylmethyl, morpholin-4-ylethyl, morpholin-4-ylpropyl, 2-morpholin-4-ylethoxy, 3-morpholin-4-ylpropoxy, 4-methylpiperazin-1-ylmethyl, 4-methylpiperazin-1 diethyl, 4-methylpiperazin-1-ylpropyl, 2-(4-methylpiperazin-1-yl)ethoxy, 3-(4-methylpiperazin-1-yl)propoxy, 1-methylpiperidin-4-ylmethoxy, 1-methylpiperidin-4-ylmethyl, 1-methylpiperidin-4-ylethyl, 1-methylpiperidin-4-ylpropyl, 2-(1-methylpiperidin-4-yl)ethoxy, 3-(1-methylpiperidin-4-yl)propoxy, pyrrolidin-1-ylpropyl, pyrrolidin-1-ylethyl, pyrrolidin-1-ylmethyl, 3-pyrrolidin-1-ylpropoxy, 2-pyrrolidin-1-ylethoxy, piperidin-1-ylmethyl, piperidin-1-ylethyl, piperidin-1-ylpropyl, 2-(piperidin-1-yl)ethoxy, 3-(piperidin-1-yl)propoxy,
R12 is fluorine, chlorine, cyano, methyl, isopropyl, trifluoromethyl or methoxy,
R13 is fluorine or chlorine,
and the salts of these compounds.

10. Compounds of formula 1 according to claim 1 in which
R1 is phenyl substituted in para position by R3 or phenyl substituted in para position by R3 and in meta position by R4,
R2 is 2-fluorophenyl, 4-fluorophenyl, 2,6-difluorophenyl, 2,4-difluorophenyl, 2-fluoro-4-methylphenyl, 2-fluoro-4-methoxyphenyl, 3-fluoro-4-methoxyphenyl, 4-methylphenyl, 4-methoxyphenyl or 3-chloro-4-fluorophenyl,
R3 is 2-morpholin-4-ylethoxy, 3-morpholin-4-ylpropoxy, 4-methylpiperazin-1-ylethoxy, 4-methylpiperazin-1-ylpropoxy, morpholin-4-ylmethyl, morpholin-4-ylethyl, morpholin-4-ylpropyl, 1-methylpiperidin-4-ylmethoxy, 2-(1-methylpiperidin-4-yl)ethoxy, 4-methylpiperazin-1-ylethyl, 3-pyrrolidin-1-ylpropoxy, 2-pyrrolidin-1-ylethoxy or 3-pyrrolidin-1-ylpropyl,
R4 is fluorine,
and the salts of these compounds.

11. Compounds of formula 1 according to claim 1 in which
R1 is phenyl substituted in para or meta position by R3,
R2 is 2-fluorophenyl, 4-fluorophenyl, 2,6-difluorophenyl, 2,4-difluorophenyl, 4-methylphenyl, 4-methoxy-phenyl or 3-chloro-4-fluorophenyl,
R3 is 2-morpholin-4-ylethoxy, 3-morpholin-4-ylpropoxy, 4-methylpiperazin-1-ylethoxy, 4-methylpiperazin-1-ylpropoxy, morpholin-4-ylmethyl, morpholin-4-ylethyl, morpholin-4-ylpropyl, 1-methylpiperidin-, 4-ylmethoxy, 2-(1-methylpiperidin-4-yl)ethoxy, 4-methylpiperazin-1-ylethyl, 3-pyrrolidin-1-ylpropoxy, 2-pyrrolidin-1-ylethoxy or 3-pyrrolidin-1-ylpropyl,
and the salts of these compounds.

12. Compounds of formula 1 according to claim 1 in which
R1 is aryl1,
aryl1 substituted by R7 and/or R8,
R9 or
R10,
R2 is phenyl,
phenyl substituted by R12 and/or R13,
naphthalenyl,
naphthalenyl substituted by R14 and/or R15,
aryl2,
aryl2 substituted by R16 and/or R17
or a radical selected from 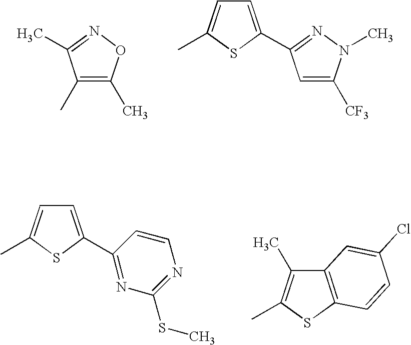
Aryl1 furanyl, thiophenyl, oxazolyl, isoxazolyl, thiazolyl, isothiazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzo[1,3]dioxolyl, 2,3-dihydrobenzo[1,4]dioxinyl, quinazolinyl, quinoxalinyl, cinnolinyl, quinolinyl, isoquinolinyl, phthalazinyl, indanyl or dibenzofuranyl,
R7 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1 C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4-C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32, wherein
R31 is hydrogen, 1-4C-alkyl or 1-4C-alkoxy-2-4C-alkyl, and
R32 is hydrogen, 1-4C-alkyl or 1-4C-alkoxy-2-4C-alkyl or wherein
R31 and R32 together and with inclusion of the nitrogen atom to which they are bonded form a pyrrolidin-, piperidin-, 4-hydroxy-piperidin-, piperazin-; 4-(1-4C-alkyl)piperazin-, [1,4]diazepan-, 4-(1-4C-alkyl)-[1,4]diazepan-, morpholin-, thiomorpholin- or an azepan-ring,
n is an integer from 0 to 4,
m is an integer from 2 to 4,
R8 is halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R9 is unsubstituted pyrrolyl, pyrazolyl, imidazolyl, indolyl, indazolyl, benzimidazolyl or benztriazolyl, or a radical selected from 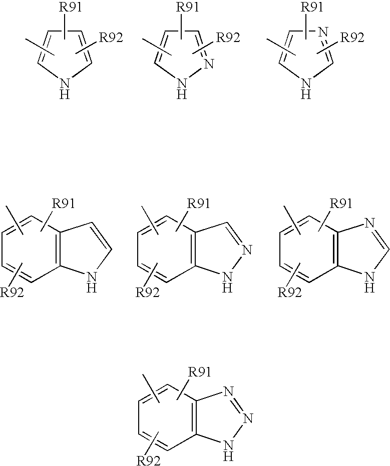
wherein
R91 is hydroxyl, halogen, cyano, carboxyl, nitro, 1-4C-alkyl, trifluoromethyl, 1-4C-alkoxy, 1-4C-alkoxy completely or predominantly substituted by fluorine, 1-4C-alkoxycarbonyl, 1-4C-alkylcarbonyl, aminocarbonyl, mono- or di-1-4C-alkylaminocarbonyl, 1-4C-alkylcarbonylamino, phenoxy, benzyloxy, 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-3-yl-1-4C-alkyl, 1-(1-4C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, 1-(1-4C-alkyl)-pyrrolidin-2-yl-1-4C-alkoxy, 1-(1-4C-alkyl)-pyrrolidin-3-yl-1-4C-alkoxy, 1-(1-4C-alkyl)-piperid-2-yl-1-4C-alkoxy, 1-(1-4C-alkyl)-piperid-3-yl-1-4C-alkoxy, 1-(1-4C-alkyl)-piperid-4-yl-1-4C-alkoxy, 1-(1-4C-alkyl)-azepan-2-yl-1-4C-alkoxy, 1-(1-4C-alkyl)-azepan-3-yl-1-4C-alkoxy, 1-(1-4C-alkyl)-azepan-4-yl-1-4C-alkoxy, —(CH₂)ₙ—N(R31)R32, —CH₂CH(OH)CH₂N(R31)R32 or —O—(CH₂)ₘ—N(R31)R32,
R92 is hydrogen, halogen, cyano, nitro, 1-4C-alkyl, trifluoromethyl, 1-4C-alkoxy or 1-4C-alkoxy completely or predominantly substituted by fluorine, amino or mono- or di-4C-alkylamino,
R10 is a radical selected from 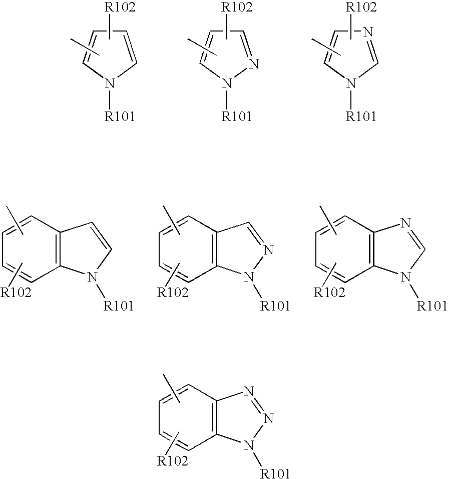
wherein
R101 is 1-4C-alkyl, 2,2,2-trifluoroethyl or 3,3,3-trifluoropropyl,
R102 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,3-dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, Indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds with the proviso that the following compounds are excluded
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]-bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide.

13. Compounds of formula 1 according to claim 1 in which
R1 is pyrid-3-yl, pyrid-4-yl, 2-methyl-2,3-dihydrobenzofuran-5-yl, benzo[1,3]dioxol-5-yl, 1-methyl-1H-pyrrol-3-yl, 4-methyl-thiophen-2-yl, 1-methyl-1H-pyrrol-2-yl, 1H-indol-5-yl, 1-methyl-1H-indol-3-yl, 1-methyl-1H-indol-5-yl, dibenzofuran-4-yl or 3,5-dimethyl-isoxazol-4-yl,
R2 is phenyl substituted by R12 and/or R13 or naphthalenyl,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or dl-1-4C-alkylamino,
and the salts of these compounds with the proviso that the following compounds are excluded
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide, and
4-Methyl-N-[4-(6-pyridin-3-yl-pyrimidin-4-ylamino)-phenyl]-benzenesulfonamide.

14. Compounds of formula 1 according to claim 1 in which
R1 is 2-methyl-2,3-dihydrobenzofuran-5-yl, benzo[1,3]dioxol-5-yl, 1-methyl-1H-pyrrol-3-yl, 4-methyl-thiophen-2-yl, 1-methyl-1H-pyrrol-2-yl, 1H-indol-5-yl, 1-methyl-1H-indol-3-yl or 1-methyl-1H-indol-5-yl.
R2 is phenyl substituted by R12 and/or R13 or naphthalenyl,
R12 is fluorine, chlorine, cyano, methyl, trifluoromethyl or methoxy,
R13 is fluorine, chlorine or methoxy,
and the salts of these compounds with the proviso that the compound
N-[4-(6-Benzo[1,3]dioxol-5-yl-pyrimidin-4-ylamino)-phenyl]-4-methyl-benzenesulfonamide is excluded.

15. Compounds of formula 1 according to claim 1 in which
R1 is 2-methyl-2,3-dihydrobenzofuran-5-yl, 1-methyl-1H-pyrrol-3-yl or 4-methylthiophen-2-yl, and
R2 is 2,6-difluorophenyl,
or
R1 is 1H-indol-5-yl, and
R2 is 2,6-difluorophenyl, 2,4-difluorophenyl, 2-fluoro-4-methylphenyl, 2-fluorophenyl or 3-fluorophenyl,
or
R1 is 1-methyl-1H-indol-5-yl or 1-methyl-1H-indol-3-yl, and
R2 is 2,6-difluorophenyl, 2-fluorophenyl or 4-methoxyphenyl,
or
R1 is 1-methyl-1H-indol-5-yl, and
R2 is 2-fluoro-4-methylphenyl,
and the salts of these compounds.

16. Compounds of formula 1 according to claim 1 in which
either
R1 is 2-methyl-2,3-dihydrobenzofuran-5-yl, 1-methyl-1H-pyrrol-3-yl or 4-methylthiophen-2-yl, and
R2 is 2,6-difluorophenyl,
or
R1 is 1H-indol-5-yl, and
R2 is 2,6-difluorophenyl, 2,4-difluorophenyl, 2-fluorophenyl or 3-fluorophenyl,
or
R1 is 1-methyl-1H-indol-5-yl or 1-methyl-1H-indol-3-yl, and
R2 is 2,6-difluorophenyl, 2-fluorophenyl or 4-methoxyphenyl,
and the salts of these compounds.

17. Compounds of formula 1 according to claim 1 in which
R1 is R11,
R2 is phenyl,
phenyl substituted by R12 and/or R13,
naphthalenyl,
naphthalenyl substituted by R14 and/or R15,
aryl2,
aryl2 substituted by R16 and/or R17
or a radical selected from 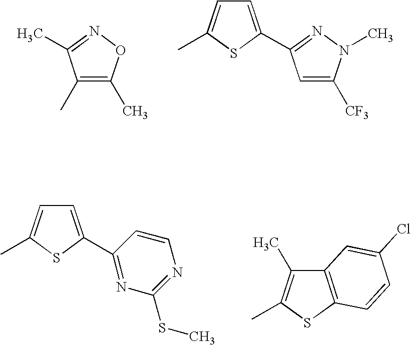
R11 is a radical selected from 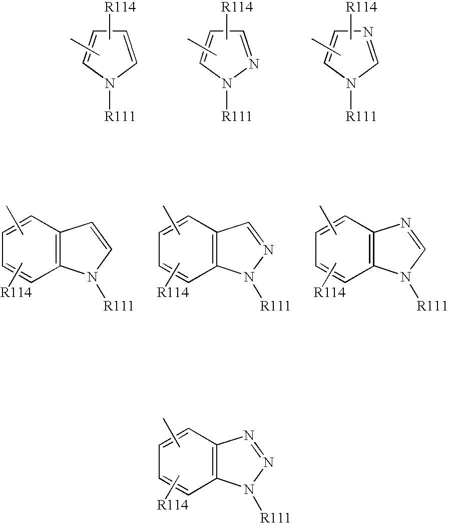
wherein
R111 is 1-(1-4C-alkyl)-pyrrolidin-2-yl-1-4C-alkyl, 1-(1C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1C-alkyl, 1-(1-4C-alkyl)-azepan-4-yl-1-4C-alkyl, —(CH₂)ₚ—N(R112)R113 or —CH₂CH(OH)CH₂N(R112)R113, wherein
R112 is hydrogen, 1-4C-alkyl or 1-4C-alkoxy-2-4C-alkyl, and
R113 is hydrogen, 1-4C-alkyl or 1-4C-alkoxy-2-4C-alkyl, or wherein
R112 and R113 together and with Inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R14 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R15 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
Aryl2 furanyl, thiophenyl, pyrrolyl, pyrazolyl, oxazolyl, isoxazolyl, thiazolyl, isothiazolyl, imidazolyl, pyridinyl, pyrimidinyl, pyrazinyl, pyridazinyl, benzofuranyl, benzothiophenyl, 2,&dihydrobenzofuranyl, benzoxazolyl, benzothiazolyl, benzimidazolyl, benzo[1,3]dioxolyl, 2,3-dihydrobenzo[1,4]-dioxinyl, quinazolinyl, quinoxalinyl, cinnolinyl, quinolinyl, isoquinolinyl, phthalazinyl, indanyl, indolyl or indazolyl,
R16 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R17 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

18. Compounds of formula 1 according to claim 1 in which
R1 is R11,
R2 is phenyl substituted by R12 and/or R13,
R11 is a radical selected from 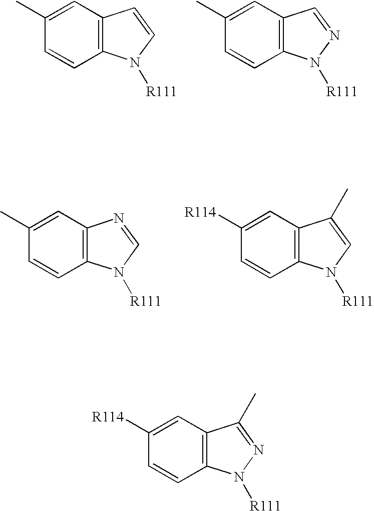
wherein
R111 is 1-(1-4C-alkyl)-pyrrolidin-2-yl-1-4C-alkyl, 1-(1C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1C-alkyl)-azepan-2-yl-1-4C-alkyl, 1-(1-4C-alkyl)-azepan-3-yl-1-4C-alkyl, 1-(1-4C-alkyl)-azepan-4-yl-1-4C-alkyl, —(CH₂)ₚ—N(R112)R113 or —CH₂CH(OH)CH₂N(R112)R113, wherein
R112 is hydrogen, 1-4C-alkyl or 1-4C-alkoxy-2-4C-alkyl, and
R113 is hydrogen, 1-4C-alkyl or 1-4C-alkoxy-2-4C-alkyl, or wherein
R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

19. Compounds of formula 1 according to claim 1 in which
R1 is R11,
R2 is phenyl substituted by R12 and/or R13,
R11 is a radical selected from 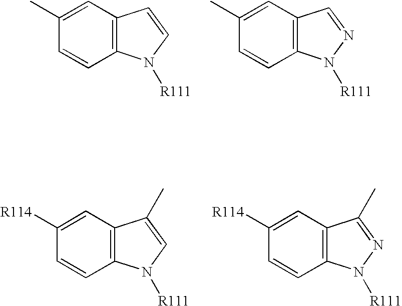
wherein
R111 is 1-(1-4C-alkyl)-pyrrolidin-2-yl-1-4C-alkyl, 1-(1-4C-alkyl)-pyrrolidin-3-yl-1-4C-alkyl, 1-(1-4C-alkyl)-piperid-2-yl-1-4C-alkyl, 1-(1-4C-alkyl)-piperid-3-yl-1-4C-alkyl, 1-(1-4C-alkyl)-piperid-4-yl-1-4C-alkyl, 1-(1-4C-alkyl)-azepan-2-yl-1-4C-alkyl, 1-(1-4C-alkyl)-azepan-3-yl-1C-alkyl, 1-(1-4C-alkyl)-azepan-4-yl-1-4C-alkyl, —(CH₂)ₚ—N(R112)R113 or —CH₂CH(OH)CH₂N(R112)R113, wherein
R112 is hydrogen, 1-4C-alkyl or 1-4C-alkoxy-2-4C-alkyl, and
R113 is hydrogen, 1-4C-alkyl or 1-4C-alkoxy-2-4C-alkyl, or wherein
R112 and R113 together and with inclusion of the nitrogen atom to which they are bonded form a pyrrolidin-, piperidin-, 4-hydroxy-piperidin-, piperazin-, 4-(1-4C-alkyl)piperazin-, [1,4]diazepan-, 4-(1-4C-alkyl)-[1,4]diazepan-, morpholin-, thiomorpholin- or an azepan-ring,
p is an integer from 1 to 4,
R114 is hydrogen, halogen, cyano, nitro, 1-4C-alkyl, trifluoromethyl, 1-4C-alkoxy or 1-4C-alkoxy completely or predominantly substituted by fluorine, amino or mono- or di-1-4C-alkylamino,
R12 is hydroxyl, halogen, cyano, nitro, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
R13 is hydroxyl, halogen, 1-4C-alkyl, trifluoromethyl, 1-4C-alkoxy, 1-4C-alkoxy which is completely or predominantly substituted by fluorine, 1-4C-alkoxycarbonyl, amino or mono- or di-1-4C-alkylamino,
and the salts of these compounds.

20. Compounds of formula 1 according to claim 1 in which
R1 is R11,
R2 is phenyl substituted by R12 and/or R13,
R11 is a radical selected from 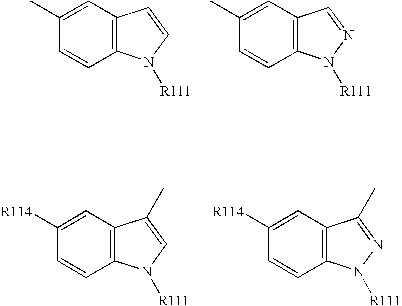
wherein
R111 is pyrrolidin-1-ylethyl, pyrrolidin-1-ylpropyl, piperidin-1-ylethyl, piperidin-1-ylpropyl, azepan-1-ylethyl, azepan-1-ylpropyl, (4-methyl-piperazin-1-yl)ethyl, (4-methyl-piperazin-1-yl)propyl, morpholin-4-ylethyl, morpholin-4-ylpropyl, (1-methyl-piperidin-4-yl)propyl, (1-methyl-piperidin-4-yl)ethyl, (1-methyl-piperidin-4-yl)methyl, dimethylaminoethyl, dimethylaminopropyl, dimethylaminobutyl, diethylaminoethyl, dimethylaminopropyl or diethylaminobutyl,
R114 is hydrogen or fluorine,
R12 is fluorine, chlorine, cyano, methyl, isopropyl, trifluoromethyl or methoxy,
R13 is fluorine or chlorine,
and the salts of these compounds.

21. Compounds of formula 1 according to claim 1 in which
R1 is R11,
R2 is 2-fluorophenyl, 4-fluorophenyl, 2,6-difluorophenyl, 2,4-difluorophenyl, 4-methylphenyl, 4-methoxy-phenyl, 3-fluoro-4-methoxyphenyl, 2-fluoro-4-methylphenyl, or 3-chloro-4-fluorophenyl,
R11 represents the following radical 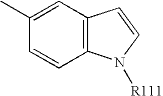
wherein
R111 is pyrrolidin-1-ylethyl, pyrrolidin-1-ylpropyl, (4-methyl-piperazin-1-yl)ethyl, (4-methyl-piperazin-1-yl)propyl, (1-methyl-piperidin-4-yl)ethyl, (1-methyl-piperidin-4-yl)methyl, dimethylaminoethyl, dimethylaminopropyl, diethylaminopropyl or diethylaminobutyl,
and the salts of these compounds.

22. Compounds according to claim 1 for use in the treatment of diseases.

23. Pharmaceutical compositions containing one or more compounds according to claim 1 together with the usual pharmaceutical auxiliaries and/or excipients.

24. Use of compounds according to claim 1 for the production of pharmaceutical compositions for the treatment of acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion Injury e.g. myocardial infarction, stroke, gut ischemia, renal failure or hemorrhage shock, or traumatic shock. The compounds according to the invention are also useful in the treatment and/or prevention of T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

25. A method for treating acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock, T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis in a patient comprising administering to said patient a therapeutically effective amount of a compound according to claim 1.

26. A compound selected from
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof for use in the treatment of diseases.

27. Pharmaceutical composition containing one or more compounds selected from
4-Methyl-N-[4-(6-naphthalen-2-yl-pyrimidin-4-ylamino)-phenyl]-benzenesulfonamide
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof together with the usual pharmaceutical auxiliaries and/or excipients.

28. Use of a compound selected from
4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamide,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof for the production of pharmaceutical compositions for the treatment of acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock. The compounds according to the invention are also useful in the treatment and/or prevention of T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

29. A method for treating acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock, T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis in a patient comprising administering to said patient a therapeutically effective amount of a compound selected from 4-Methyl-N-[4-(6-naphthalen-2-yl-pyrimidin-4-ylamino)-phenyl]-benzenesulfonamide,
N-{4-[6-(Bis-trifluoromethyl-phenyl)-pyrimidin-4-ylamino]-phenyl}-4-methyl-benzenesulfonamide,
4-Methyl-N-[4-(6-phenyl-pyrimidin-4-ylamino)-phenyl]-benzenesulfonamide,
4-Methyl-N-[4-(6-naphthalen-1-yl-pyrimidin-4-ylamino)-phenyl]-benzenesulfonamido,
N-{4-[6-(2-Benzyloxy-phenyl)-pyrimidin-4-ylamino]-phenyl}-4-methyl-benzenesulfonamide,
N-{4-[6-(4-Benzyloxy-phenyl)-pyrimidin-4-ylamino]-phenyl}-4-methyl-benzenesulfonamide,
N-{4-[6-(3,4-Dimethoxy-phenyl)-pyrimidin-4-ylamino]-phenyl}-4-methyl-benzenesulfonamide,
N-{4-[6-(4-Hydroxy-phenyl)-pyrimidin-4-ylamino]-phenyl}-4-methyl-benzenesulfonamide,
N-(3-{6-[4-(Toluene-4-sulfonylamino)-phenylamino]-pyrimidin-4-yl}-phenyl)-acetamide,
N-{4-[6-(2-Methoxy-phenyl)-pyrimidin-4-ylamino]-phenyl}-4-methyl-benzenesulfonamide,
4-Methyl-N-{4-[6-(3-nitro-phenyl)-pyrimidin-4-ylamino]-phenyl}-benzenesulfonamide,
4-Methyl-N-{4-[6-(4-trifluoromethoxy-phenyl)-pyrimidin-4-ylamino]-phenyl}-benzenesulfonamide,
N-{4-[6-(4-Cyano-phenyl)-pyrimidin-4-ylamino]-phenyl}-4-methyl-benzenesulfonamide,
4-Methyl-N-{4-[6-(4-morpholin-4-yl-phenyl)-pyrimidin-4-ylamino]-phenyl}-benzenesulfonamide,
N-{4-[6-(3-Amino-phenyl)-pyrimidin-4-ylamino]-phenyl}-4-methyl-benzenesulfonamide,
N-{4-[6-(3-Dimethylamino-phenyl)-pyrimidin-4-ylamino]-phenyl}-4-methyl-benzenesulfonamide,
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamido,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof.

30. A compound selected from
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide,
or a salt thereof for use in the treatment of diseases.

31. Pharmaceutical compositions containing one or more compounds selected from N-[4-(6-Benzo[1,3]dioxol-5-yl-pyrimidin-4-ylamino)-phenyl]-4-methyl-benzenesulfonamide,
4-Methyl-N-[4-(6-pyridin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof together with the usual pharmaceutical auxiliaries and/or excipients.

32. Use of compounds selected from
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide,
4-Methyl-N-[4-(6-quinolin-8-yl-pyrimidin-4-ylamino)-phenyl]-benzenesulfonamide,
or a salt thereof for the production of pharmaceutical compositions for the treatment of acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial Infarction, stroke, gut ischemia, renal failure or hemorrhage shock, or traumatic shock. The compounds according to the invention are also useful in the treatment and/or prevention of T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis.

33. A method for treating acute or chronic rejection of organ or tissue allo- or xenografts, atherosclerosis, vascular occlusion due to vascular injury such as angioplasty, restenosis, hypertension, heart failure, chronic obstructive pulmonary disease, CNS diseases such as Alzheimer disease or amyotrophic lateral sclerosis, cancer, infectious diseases such as AIDS, septic shock or adult respiratory distress syndrome, ischemia/reperfusion injury e.g. myocardial infarction, stroke, gut ischemia, renal failure or hemorrhage shock, or traumatic shock, T-cell mediated acute or chronic inflammatory diseases or disorders or autoimmune diseases e.g. rheumatoid arthritis, osteoarthritis, systemic lupus erythematosus, Hashimoto's thyroidis, multiple sclerosis, myasthenia gravis, diabetes type I or II and the disorders associated therewith, respiratory diseases such as asthma or inflammatory lung injury, inflammatory liver injury, inflammatory glomerular injury, cutaneous manifestations of immunologically-mediated disorders or illnesses, inflammatory and hyperproliferative skin diseases (such as psoriasis, atopic dermatitis, allergic contact dermatitis, irritant contact dermatitis and further eczematous dermatitises, seborrhoeic dermatitis), inflammatory eye diseases, e.g. Sjoegren's syndrome, keratoconjunctivitis or uveitis, inflammatory bowel disease, Crohn's disease or ulcerative colitis in a patient comprising administering to said patient a therapeutically effective amount of a compound selected from
N-[4-(6-Benzo[1,3]dioxol-5-yl-pyrimidin-4-ylamino)-phenyl]-4-methyl-benzenesulfonamide,
4-Methyl-N-[4-(6-pyridin-4-yl-pyrimidin-4-ylamino)-phenyl]-benzenesulfonamide,
N-[4-(2′,4′-Dimethoxy-[4,5′]bipyrimidinyl-6-ylamino)-phenyl]-4-methyl-benzenesulfonamide,
4-Methyl-N-[4-(6-pyridin-3-yl-pyrimidin-4-ylamino)-phenyl]-benzenesulfonamide,
N-[4-(6-Benzofuran-2-yl-pyrimidin-4-ylamino)-phenyl]-4-methyl-benzenesulfonamide,
4-Methyl-N-[4-(6-thiophen-3-yl-pyrimidin-4-ylamino)-phenyl]-benzenesulfonamide,
N-[4-(6-Dibenzofuran-4-yl-pyrimidin-4-ylamino)-phenyl]-4-methyl-benzenesulfonamide,
N-[4-(6-Benzo[b]thiophen-2-yl-pyrimidin-4-ylamino)-phenyl]-4-methyl-benzenesulfonamide, and
4-Methyl-N-[4-(6-quinolin-8-yl-pyrimidin-4-ylamino)-phenyl]-benzenesulfonamide,
or a salt thereof.
